(19) United States
(12) Patent Application Publication
(10) Pub. No.: US 2026/0176629 A1
(43) Pub. Date: Jun. 25, 2026

(54) METHODS AND COMPOSITIONS FOR TREATING EPILEPSY

(71) Applicant: uniQure France, Illkirch-Graffenstaden (FR)

(72) Inventors: Nick PEARSON, Illkirch-Graffenstaden (FR); Stephane BAUDOUIN, Illkirch-Graffenstaden (FR); Andreas BORTA, Illkirch-Graffenstaden (FR); Richard PORTER, Binningen (CH)

(21) Appl. No.: 19/289,942

(22) Filed: Aug. 4, 2025

Related U.S. Application Data
(63) Continuation of application No. 19/120,074.
(60) Provisional application No. 63/378,957, filed on Oct. 10, 2022.
(60) Provisional application No. 63/511,314, filed on Jun. 30, 2023.

(51) Int. Cl. C12N 15/113

(52) U.S. Cl. CPC C12N 15/113; C12N 2310/14

(57) ABSTRACT

Disclosed are methods and compositions relating to antisense therapy for treating epilepsy, such as a focal epilepsy and temporal lobe epilepsy, in a subject in need thereof by targeting GRIK2 mRNA. In particular, the disclosure provides methods for treating symptoms (e.g., seizures) of epilepsy in a subject by administering a particular dose in a defined volume and in a specific route of administration of an inhibitory ribopolynucleotide or adeno-associated viral vector encoding the same, which is capable of inhibiting expression of GRIK2.

### FIELD OF THE DISCLOSURE

The disclosure is in the field of epilepsy. In particular, the disclosure relates to methods and compositions for treating an epilepsy, such as, e.g., temporal lobe epilepsy or focal epilepsy.

### SEQUENCE LISTING

The instant application contains a Sequence Listing which has been submitted electronically in XML format and is hereby incorporated by reference in its entirety. Said XML copy, created on Aug. 4, 2025, is named 51460-012004_Sequence_Listing_8_4_25 and is 50,563 bytes in size.

### BACKGROUND

Globally, an estimated 5 million people are diagnosed each year with epilepsy, a neurological disorder marked by seizures, or sudden recurrent episodes of sensory disturbance, loss of consciousness, or convulsions associated with abnormal electrical activity in the brain. A typical diagnosis of epilepsy arises when a patient experiences two or more unprovoked seizures. Causes of epilepsy include genetic abnormalities, prior brain infection, prenatal injuries, developmental disorders, and other neurological issues such as strokes or brain tumors, though approximately 50% of people who are diagnosed with epilepsy have no known cause for the development of the disorder.

Temporal lobe epilepsy (TLE) is the most common form of partial epilepsy in adults (30-40% of all forms of epilepsies). It is well established that the hippocampus plays a key role in the pathophysiology of TLE. In human patients and animal models of TLE, an aberrant rewiring of neuronal circuits occurs. One of the best examples of network reorganization (“reactive plasticity”) is the sprouting of recurrent mossy fibers (rMF) that establish novel pathophysiological glutamatergic synapses onto dentate granule cells (DGCs) in the hippocampus (Tauck and Nadler, 1985; Represa et al., 1989a, 1989b; Sutula et al., 1989; Gabriel et al., 2004) leading to a recurrent excitatory loop. rMF synapses operate through ectopic kainate receptors (KARs) (Epsztein et al., 2005; Artinian et al., 2011, 2015). KARs are tetrameric glutamate receptors assembled from GluK1-GluK5 subunits. In heterologous expression systems, GluK1, GluK2, and GluK3 may form homomeric receptors, while GluK4 and GluK5 form heteromeric receptors in conjunction with GluK1-3 subunits. Native KARs are widely distributed in the brain with high densities of receptors found in the hippocampus (Carta et al, 2016, EJN), a key structure involved in TLE. Prior studies by the present inventors have established that epileptic activities including interictal spikes and ictal discharges were markedly reduced in mice lacking the GluK2 KAR subunit. Moreover, epileptiform activities were strongly reduced following the use of pharmacological small molecule antagonists of GluK2/GluK5-containing KARs, which block ectopic synaptic KARs (Peret et al., 2014). These data support a hypothesis that KARs ectopically expressed at rMFs in DGCs play a major role in chronic seizures in TLE. Therefore, aberrant KARs expressed in DGCs and composed of GluK2/GluK5 are considered to represent a promising target for the treatment of pharmaco-resistant epilepsies such as TLE.

RNA interference (RNAi) strategies have been proposed for many disease targets. Successful application of RNAi-based therapies has been limited. RNAi therapeutics face multiple challenges, such as the need for repeat dosing and formulation challenges. However, available RNAi-based gene therapies for the treatment of intractable TLE are limited. Therefore, there exists an urgent need for new therapeutic modalities for the treatment of seizure disorders, such as, e.g., TLE (e.g., TLE refractory to treatment).

### SUMMARY OF THE DISCLOSURE

The disclosure provides compositions and methods for the treatment or prevention of an epilepsy, such as, e.g., a temporal lobe epilepsy (TLE), in a subject (e.g., a human) in need thereof. The disclosed methods include administration of a therapeutically effective amount of an inhibitory ribopolynucleotide that targets an mRNA encoded by a glutamate ionotropic receptor kainate type subunit 2 (GRIK2) gene, or a nucleic acid vector encoding the same (e.g., a lentiviral vector or an adeno-associated viral (AAV) vector, such as, e.g., an AAV9 vector), to a subject diagnosed with or displaying one or more (e.g., two, three, four, or more) symptoms of epilepsy. The disclosed polynucleotides exhibit improved loading into the RNA-induced silencing complex (RISC) protein in order to enhance RNA-interference-mediated degradation of the GRIK2 transcript. The disclosure also features pharmaceutical compositions containing one or more of the disclosed inhibitory ribopolynucleotides and AAV vectors encoding the same.

In a first aspect, the disclosure provides a method of treating epilepsy in a human subject in need thereof by administering to the subject a vector (e.g., a viral vector, such as, e.g., an adeno-associated viral (AAV) vector (e.g., an AAV9 vector)) encoding a ribopolynucleotide that inhibits glutamate ionotropic receptor kainate type subunit 2 (GRIK2), in which the vector (e.g., an AAV vector) is administered to the subject intra-parenchymally in an amount of from about 1×10⁹ vector genomes (vg) to about 1×10¹⁴ vg and in which the ribopolynucleotide includes a nucleic acid sequence with at least 95% sequence identity to SEQ ID NO: 2, 7, 8 or 16. In an embodiment, the vector is administered in an amount of from about 1×10⁹ vg/mL to about 1×10¹⁴ vg/mL (e.g., 1×10¹¹ vg/mL to about 1.0×10¹³ vg/mL), e.g., in a volume of 3.0 mL or less (e.g., about 2.0 mL or less, such as about 1.8 mL or less). In another embodiment, the administration is provided to one or each hemisphere of the brain of the subject (e.g., within one or each hemisphere of the hippocampus of the subject). In another embodiment, the vector is administered to the subject in a volume of at least 0.1 mL or greater (e.g., at least 0.2 mL, 0.3 mL, 0.4 mL, or 0.5 mL or greater). In another embodiment, the ribopolynucleotide has at least 96%, 97%, 98%, or 99% sequence identity to, or the sequence identity of, any one of SEQ ID NOs: 2, 7, 8, or 16 (e.g., SEQ ID NO: 2 or 16, such as SEQ ID NO: 2).

In an embodiment of the foregoing aspect, the ribopolynucleotide includes the nucleic acid sequence of SEQ ID NO: 2.

In an embodiment, the ribopolynucleotide includes the nucleic acid sequence of SEQ ID NO: 16.

In an embodiment, the ribopolynucleotide includes the nucleic acid sequence of SEQ ID NO: 7.

In an embodiment, the ribopolynucleotide includes the nucleic acid sequence of SEQ ID NO: 8.

In several embodiments of the foregoing aspect, the subject is less than 65 years of age (e.g., the subject is aged 5 years to 65 years old, or, e.g., a child (e.g., aged less than 10 years old or younger), an adolescent (e.g., aged 10 years old to less than 19 years old), or an adult (e.g., aged 19-65 years old)). In some embodiments, the subject is diagnosed with or exhibits one or more symptoms of epilepsy, in which:
(a) the subject has or experiences:
   (i) at least 12 documented seizures during the previous 90 days, wherein the seizures include:
      at least 2 documented focal impaired awareness seizures; and/or
      at least 10 documented focal aware seizures;
   (ii) no 21-day seizure-free period in the previous 90 days;
   (iii) hippocampal atrophy, for example, as determined by MRI-T1 imaging, optionally with:
      increased ipsilateral mesial signal on T2 imaging; or
      ipsilateral hypometabolism on fluorodeoxyglucose positron emission tomography (FDG-PET);
      (iv) a score of 23 or above on a Montreal Cognitive Assessment (MoCA); and/or
      (v) no significant focal neurocognitive dysfunction, inconsistent with disease pathology-related magnetic resonance imaging (MRI) and positron emission tomography (PET) imaging findings; and/or
(b) the subject does not have:
   (i) lesions on neuroimaging outside of the mesial temporal love area, temporal neocortical or extratemporal lesions on MRI, or diffuse unilateral or bilateral hypometabolism on PET;
   (ii) a progressive neurological disorder;
   (iii) a psychogenic seizure within the last 2 years;
   (iv) an implanted device that would contraindicate MRI-guided convection-enhanced delivery (CED), such as a vagus nerve stimulation (VNS) device or a cochlear implant;
   (vi) a major disease-unrelated neurosurgical intervention due to intracranial tumor, trauma, or bleeding; and/or
   (vii) a medical history of schizophrenia.

In several embodiments, the one or more symptoms of epilepsy include a recurrent epileptic seizure that is refractory to treatment, wherein, optionally, the seizure is a focal seizure, a generalized seizure, or a febrile seizure. In some embodiments, the epilepsy is focal epilepsy (FE) or temporal lobe epilepsy (TLE).

In some embodiments of the foregoing aspect, administration of the vector (e.g., an AAV vector) reduces the level of GRIK2 expression in a transduced cell in the hippocampus of the subject by at least 10%, at least at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or more (e.g., 100%) relative to a control vector (e.g., a control AAV vector) or relative to a cell in the brain (e.g., the hippocampus) of the subject that is not transduced. In some embodiments, administration of the vector (e.g., the AAV vector) reduces the level of GRIK2 expression in a transduced cell in the brain (e.g., the hippocampus) of the subject by between 5% to 100%, such as between 10% to 100%, 20% to 100%, 30% to 100%, 40% to 100%, 50% to 100%, 60% to 100%, 70% to 100%, 80% to 100%, 90% to 100%, 10% to 90%, 10% to 80%, 10% to 70%, 10% to 60%, 10% to 50%, 10% to 40%, 10% to 30%, 10% to 20%, 20% to 90%, 20% to 80%, 20% to 70%, 20% to 60%, 20% to 50%, 30% to 90%, 30% to 80%, 30% to 70%, 30% to 60%, 30% to 50%, 40% to 90%, 40% to 80%, 40% to 70%, 40% to 60%, 40% to 50%, or by 30%, e.g., each relative to a control vector (e.g., a control AAV vector) or relative to a cell in the brain (e.g., the hippocampus) of the subject that is not transduced. In some embodiments, the level of GRIK2 is reduced for at least 28 days, at least 30 days, at least 60 days, at least 90 days, at least 120 days, at least 150 days, at least 180 days, or at least 365 days, at least 2 years, 3 years, 4 years, 5 years, 10 years, 15 years, or 20 years, or for the life of the subject.

In some embodiments of the foregoing aspect, administration of the vector (e.g., an AAV vector) reduces the level of GluK2 protein in a transduced cell in the brain (e.g., the hippocampus) of the subject by at least 10%, at least at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or more (e.g., 100%) relative to a control vector (e.g., a control AAV vector) or relative to a cell in the brain (e.g., the hippocampus) of the subject that is not transduced. In some embodiments, administration of the AAV vector reduces the level of GluK2 in a transduced cell in the hippocampus of the subject by between 5% to 100%, such as between 10% to 100%, 20% to 100%, 30% to 100%, 40% to 100%, 50% to 100%, 60% to 100%, 70% to 100%, 80% to 100%, 90% to 100%, 10% to 90%, 10% to 80%, 10% to 70%, 10% to 60%, 10% to 50%, 10% to 40%, 10% to 30%, 10% to 20%, 20% to 90%, 20% to 80%, 20% to 70%, 20% to 60%, 20% to 50%, 30% to 90%, 30% to 80%, 30% to 70%, 30% to 60%, 30% to 50%, 40% to 90%, 40% to 80%, 40% to 70%, 40% to 60%, 40% to 50%, or by 30%, e.g., each relative to a control AAV vector or relative to a cell in the brain (e.g., the hippocampus) of the subject that is not transduced. In some embodiments, the level of GluK2 is reduced for at least 28 days, at least 30 days, at least 60 days, at least 90 days, at least 120 days, at least 150 days, at least 180 days, or at least 365 days, at least 2 years, 3 years, 4 years, 5 years, 10 years, 15 years, or 20 years, or for the life of the subject.

In some embodiments of the foregoing aspect, the vector (e.g., the AAV vector) is administered to the subject in an amount of about 1×10⁹ vg, 2×10⁹ vg, 3×10⁹ vg, 4×10⁹ vg, 5×10⁹ vg, 6×10⁹ vg, 7×10⁹ vg, 8×10⁹ vg, 9×10⁹ vg, 1×10¹⁰ vg, 2×10¹⁰ vg, 3×10¹⁰ vg, 4×10¹⁰ vg, 5×10¹⁰ vg, 6×10¹⁰ vg, 7×10¹⁰ vg, 8×10¹⁰ vg, 9×10¹⁰ vg, 1×10¹¹ vg, 2×10¹¹ vg, 3×10¹¹ vg, 4×10¹¹ vg, 5×10¹¹ vg, 6×10¹¹ vg, 7×10¹¹ vg, 8×10¹¹ vg, 9×10¹¹ vg, 1×10¹² vg, 2×10¹² vg, 3×10¹² vg, 4×10¹² vg, 5×10¹² vg, 6×10¹² vg, 7×10¹² vg, 8×10¹² vg, 9×10¹² vg, or 1×10¹³ vg.

In some embodiments of the foregoing aspect, the vector (e.g., the AAV vector) is administered to the subject in an amount of about 1×10¹¹ vg/mL, 2×10¹¹ vg/mL, 3×10¹ vg/mL, 4×10¹ vg/mL, 5×10¹¹ vg/mL, 6×10¹¹ vg/mL, 7×10¹¹ vg/mL, 8×10¹¹ vg/mL, 9×10¹¹ vg/mL, 1×10¹² vg/mL, 2×10¹² vg/mL, 3×10¹² vg/mL, 4×10¹² vg/mL, 5×10¹² vg/mL, 6×10¹² vg/mL, 7×10¹² vg/mL, 8×10¹² vg/mL, 9×10¹² vg/mL, or 1×10¹³ vg/mL. In some embodiments, the vector (e.g., the AAV vector) is administered to the subject in an amount of from about 3×10⁸ vg/mm³ brain (e.g., about 3×10⁸ vg/mm³ hippocampus) to about 1.2×10⁹ vg/mm³ brain (e.g., about 1.2×10⁹ vg/mm³ hippocampus). In some embodiments, the vector (e.g., the AAV vector) is administered to the subject in an amount of from about 9×10¹¹ total vg/brain (e.g., about 9×10¹¹ total vg/hippocampus) to about 3.6×10¹² total vg/brain (e.g., about 3.6×10¹² total vg/hippocampus).

In some embodiments of the foregoing aspect, the vector (e.g., the AAV vector) is administered to the subject in a single dose per hemisphere of the brain (e.g., the hippocampus) including the amount, and wherein the vector (e.g., the AAV vector) is administered by advancing a needle through the brain (e.g., the hippocampus) at between 1-10, between 2-9, between 3-8, between 4-7, or between 5-6 focal sites within the brain (e.g., the hippocampus), in which the total volume of the single dose is divided by the number of focal sites, and wherein, e.g., the focal sites are determined by a magnetic resonance imaging (MRI) or positron emission tomography (PET) scan. In some embodiments of the foregoing aspect, the vector (e.g., the AAV vector) is administered to the subject in a single dose per hemisphere of the brain (e.g., the hippocampus) including the amount, in which the vector (e.g., the AAV vector) is administered by advancing a needle through the brain (e.g., the hippocampus) at five or fewer focal sites within the brain (e.g., the hippocampus), in which the volume of the single dose is divided by the number of focal sites. In some embodiments, the vector (e.g., the AAV vector) is administered to the subject in a volume of about 0.1 mL to about 3.0 mL (e.g., about 0.5 mL to 2.5 mL, such as about 0.5 mL to 2.0 mL, 0.5 mL to 1.8 mL, about 0.75 mL to 1.5 mL, about 1 mL to 1.25 mL, or about 1.15 mL).

In some embodiments of the foregoing aspect, the ribopolynucleotide includes a nucleic acid sequence that encodes miR3bR and miR38R (e.g., SEQ ID NO: 13 or 16, or a variant thereof with at least 95% sequence identity thereto), miR3bR (e.g., SEQ ID NO: 14, or a variant thereof with at least 95% sequence identity thereto), or miR38R (e.g., SEQ ID NO: 15, or a variant thereof with at least 95% sequence identity thereto). For example, following administration of the vector (e.g., the AAV vector), a cell of the subject expresses about 1×10³ to 1×10¹⁰ copies/nanogram of RNA of both miR3bR and miR38R (e.g., SEQ ID NO: 13 or 16, or a variant thereof with at least 95% sequence identity thereto), such as about 1×10³ to 1×10⁹ copies/nanogram of RNA of both miR3bR and miR38R, about 1×10³ to 1×10⁸ copies/nanogram of RNA of both miR3bR and miR38R, or about 1×10³ to 1×10⁷ copies/nanogram of RNA of both miR3bR and miR38R.

In some embodiments of the foregoing aspect, the vector (e.g., the AAV vector) is expressed in a cell of the brain of the subject (e.g., a cell of the hippocampus of the subject). In an embodiment, the cell is a hippocampal neuron, such as a dentate granule cell (DGC) or a glutamatergic pyramidal neuron. In some embodiments, expression of the vector (e.g., the AAV vector) does not occur in peripheral tissues (e.g., liver and heart) of the subject or occurs at a level of 10% or less relative to expression in, e.g., a transduced neuron. In some embodiments, expression of the vector (e.g., the AAV vector) in the subject's dorsal root ganglion (DRG), blood, and/or cerebral spinal fluid (CSF) occurs at a level of 10% or less relative to expression in the hippocampal neuron. In some embodiments, expression of the vector (e.g., the AAV vector) in the subject's DRG, blood, and/or CSF occurs at a level of 1×10⁶ double stranded (ds) vg/μg of DNA or ds vg/mL biofluid or less (e.g., 1×10⁵ ds vg/μg of DNA or ds vg/mL biofluid, 1×10⁴ ds vg/μg of DNA or ds vg/mL biofluid, 1×10³ ds vg/μg of DNA or ds vg/mL biofluid, 1×10² ds vg/μg of DNA or ds vg/mL biofluid, 10 ds vg/μg of DNA or ds vg/mL biofluid, or less). In some embodiments, expression of the vector (e.g., the AAV vector) in the subject's DRG, blood, and/or CSF occurs at a level of 1×10⁵ ds vg/μg of DNA or ds vg/mL biofluid or less. In some embodiments, expression of the vector (e.g., the AAV vector) in the subject's DRG, blood, and/or CSF occurs at a level of 1×10⁴ ds vg/μg of DNA or ds vg/mL biofluid or less. In some embodiments, expression of the vector (e.g., the AAV vector) in the subject's DRG, blood, and/or CSF occurs at a level of 1×10³ ds vg/μg of DNA or ds vg/mL biofluid or less. In some embodiments, expression of the vector (e.g., the AAV vector) in the subject's DRG, blood, and/or CSF occurs at a level of 1×10² ds vg/μg of DNA or ds vg/mL biofluid or less. In some embodiments, expression of the vector (e.g., the AAV vector) in the subject's DRG, blood, and/or CSF occurs at a level of 10 ds vg/μg of DNA or ds vg/mL biofluid or less.

In some embodiments of the foregoing aspect, the method:
(a) reduces the number of seizures per day and/or reduces epileptiform discharges in the subject, for example, as measured by an electroencephalogram and standardized to seizure frequency per 30 days;
(b) improves the subject's measurements on routine laboratory parameters, such as hematology, biochemistry, coagulation, and urinalysis parameters, within at least 4 weeks, at least 5 weeks, at least 6 weeks, at least 7 weeks, at least 8 weeks, at least 3 months, at least 4 months, at least 5 months, at least 6 months, or at least 1 year after administration of the vector (e.g., the AAV vector);
(c) reduces frequency of interictal discharges, as measured by an electroencephalogram;
(d) reduces aberrant neurological behavior by the subject; and/or
(e) produces no adverse effects after 4 weeks or 8 weeks.

In some embodiments of the foregoing aspect, the vector (e.g., the AAV vector) is administered once per year (e.g., once per 2, 3, 4, 5, 6, 7, 8, 9, 10, 15, 20, 25, or 30 years). In some embodiments, the vector (e.g., the AAV vector) is administered to the subject once in their lifetime.

In a second aspect, the disclosure provides a kit that includes a container with a viral vector (e.g., an AAV vector (e.g., an AAV9 vector)) encoding a ribopolynucleotide including a nucleic acid sequence with at least 95%, at least 96%, at least 97%, at least 98%, at least 99%, or more sequence identity to any one of SEQ ID NOs: 2, 7, 8, and 16 (e.g., SEQ ID NOs: 2 and 16, such as SEQ ID NO: 2) formulated for intra-parenchymal injection, in which the container comprises an amount of the vector (e.g., the AAV vector) of from about 1×10⁹ vg to about 1.0×10¹³ vg (e.g., about 1×10¹⁰ vg, about 1×10¹¹ vg, or about 1×10¹² vg), such as from about 1×10⁹ vg/mL to about 1.0×10¹³ vg/mL (e.g., about 1×10¹⁰ vg/mL, about 1×10¹¹ vg/mL, or about 1×10¹² vg/mL). In an embodiment, the container includes a volume of 3.0 mL or less (e.g., about 2.5 mL, about 2.0 mL, about 1.8 mL, about 1.5 mL, about 1.2 mL, about 1.1 mL, about 1.0 mL, about 0.9 mL, about 0.8 mL, about 0.7 mL, about 0.6 mL, about 0.5 mL, about 0.4 mL, about 0.3 mL, about 0.2 mL, or about 0.1 mL). Optionally, the kit further includes a package insert with instructions for administering the vector (e.g., the AAV vector) to the subject in accordance with the first aspect of the disclosure. In some embodiments, the ribopolynucleotide includes the nucleic acid sequence of SEQ ID NO: 2, 7, 8, or 16 (e.g., SEQ ID NO: 2 or 16, such as SEQ ID NO: 2).

In a third aspect, the disclosure provides a composition including a vector (e.g., a viral vector, such as an AAV vector (e.g., an AAV9 vector)) with a nucleic acid molecule including a nucleic acid sequence with at least 95%, at least 96%, at least 97%, at least 98%, at least 99%, or more sequence identity to SEQ ID NO: 2, 7, 8, or 16 (e.g., SEQ ID NO: 2 or 16, such as SEQ ID NO: 2), in which the vector (e.g., the AAV vector) is present in the composition in an amount of from about 1×10⁹ vg to about 1.0×10¹³ vg (e.g., about 1×10¹⁰ vg, about 1.0×10¹¹ vg, or about 1.0×10¹² vg), such as, e.g., about 1×10⁹ vg/mL to about 1.0×10¹³ vg/mL (e.g., about 1×10¹⁰ vg/mL, about 1.0×10¹¹ vg/mL, or about 1.0×10¹² vg/mL). The composition includes, e.g., a volume of 3.0 mL or less (e.g., about 2.5 mL, about 2.0 mL, about 1.8 mL, about 1.5 mL, about 1.2 mL, about 1.1 mL, about 1.0 mL, about 0.9 mL, about 0.8 mL, about 0.7 mL, about 0.6 mL, about 0.5 mL, about 0.4 mL, about 0.3 mL, about 0.2 mL, or about 0.1 mL). In an embodiment, the composition is formulated for intra-parenchymal administration. In some embodiments, the nucleic acid molecule includes the nucleic acid sequence of SEQ ID NO: 2, 7, 8, or 16 (e.g., SEQ ID NO: 2 or 16, such as SEQ ID NO: 2).

In a fourth aspect, the disclosure provides a composition for use in treating epilepsy (e.g., a focal epilepsy, such as TLE), in which the composition includes a vector (e.g., a viral vector, such as an AAV vector (e.g., an AAV9 vector)) encoding a ribopolynucleotide including a nucleic acid sequence with at least 95%, at least 96%, at least 97%, at least 98%, at least 99%, or more sequence identity to SEQ ID NO: 2, 7, 8, or 16 (e.g., SEQ ID NO: 2 or 16, such as SEQ ID NO: 2) that inhibits GRIK2 in a cell of a subject. The composition is formulated for intra-parenchymal administration, contains an amount of from about 1×10⁹ vg to about 1.0×10¹³ vg (e.g., about 1×10¹⁰ vg, about 1.0×10¹¹ vg, or about 1.0×10¹² vg), such as about 1×10⁹ vg/mL to about 1.0×10¹³ vg/mL (e.g., about 1.0×10¹⁰ vg/mL, about 1.0×10¹¹ vg/mL, or about 1.0×10¹² vg/mL) of the vector (e.g., the AAV vector). In an embodiment, the composition has a volume of 3.0 mL or less (e.g., about 2.5 mL, about 2.0 mL, about 1.8 mL, about 1.5 mL, about 1.2 mL, about 1.1 mL, about 1.0 mL, about 0.9 mL, about 0.8 mL, about 0.7 mL, about 0.6 mL, about 0.5 mL, about 0.4 mL, about 0.3 mL, about 0.2 mL, or about 0.1 mL). In some embodiments, the ribopolynucleotide includes the nucleic acid sequence of SEQ ID NO: 2, 7, 8, or 16 (e.g., SEQ ID NO: 2 or 16, such as SEQ ID NO: 2). In some embodiments, the composition has a volume of about 0.5 mL to 1.8 mL, about 0.75 mL to 1.5 mL, about 1 mL to 1.25 mL, or about 1.15 mL.

In some embodiments of any of the foregoing aspects, the subject is less than 65 years of age (e.g., the subject is aged 5 years to 65 years old, or, e.g., a child (e.g., aged less than 10 years old or younger), an adolescent (e.g., aged 10 years old to less than 19 years old), or an adult (e.g., aged 19-65 years old)). In some embodiments, the subject is diagnosed with or exhibits one or more symptoms of epilepsy. For example, the subject:
(a) has or experiences:
   (i) at least 12 documented seizures during the previous 90 days, wherein the seizures include:
      at least 2 documented focal impaired awareness seizures; and/or
      at least 10 documented focal aware seizures;
   (ii) no 21-day seizure-free period in the previous 90 days;
   (iii) hippocampal atrophy, for example, as determined by MRI-T1 imaging, optionally with:
      increased ipsilateral mesial signal on T2 imaging; or
      ipsilateral hypometabolism on FDG-PET;
   (iv) a score of 23 or above on a MoCA; and/or
   (v) no significant focal neurocognitive dysfunction, inconsistent with disease pathology-related MRI and PET imaging findings; and/or
(b) does not have:
   (i) lesions on neuroimaging outside of the mesial temporal love area, temporal neocortical or extratemporal lesions on MRI, or diffuse unilateral or bilateral hypometabolism on PET;
   (ii) a progressive neurological disorder;
   (iii) a psychogenic seizure within the last 2 years;
   (iv) an implanted device that would contraindicate MRI-guided CED, such as a VNS device or a cochlear implant;
   (vi) a major disease-unrelated neurosurgical intervention due to intracranial tumor, trauma, or bleeding; and/or
   (vii) a medical history of schizophrenia.

In some embodiments of any of the foregoing aspects, the one or more symptoms of epilepsy include recurrent epileptic seizures that are refractory to treatment. Optionally, the seizures are focal seizures, generalized seizures, or febrile seizures. In some embodiments, the epilepsy is FE or TLE.

In some embodiments of any of the foregoing aspects, administration of the vector (e.g., the AAV vector) reduces the level of GRIK2 expression in a transduced cell in the hippocampus of the subject by at least 10%, at least at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or more (e.g., 100%), e.g., relative to a control vector (e.g., a control AAV vector) or relative to a cell in the brain (e.g., a cell in the hippocampus) of the subject that is not transduced. In some embodiments, administration of the vector (e.g., the AAV vector) reduces the level of GRIK2 expression in a transduced cell in the brain (e.g., a cell in the hippocampus) of the subject by between 5% to 60%, by between 10% to 50%, by between 20% to 40%, or by 30%, e.g., relative to a control AAV vector or relative to a cell in the hippocampus of the subject that is not transduced. In some embodiments, the level of GRIK2 is reduced for at least 28 days, at least 30 days, at least 60 days, at least 90 days, at least 120 days, at least 150 days, at least 180 days, or at least 365 days, at least 2 years, 3 years, 4 years, 5 years, 10 years, 15 years, or 20 years, or for the life of the subject.

In some embodiments of any of the foregoing aspects, administration of the vector (e.g., the AAV vector) reduces the level of GluK2 protein in a transduced cell in the brain (e.g., a cell in the hippocampus) of the subject by at least 10%, at least at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or more (e.g., 100%), e.g., relative to a control AAV vector or relative to a cell in the hippocampus of the subject that is not transduced. In some embodiments, administration of the vector (e.g., the AAV vector) reduces the level of GluK2 in a transduced cell in the brain (e.g., the hippocampus) of the subject by between 5% to 60%, by between 10% to 50%, by between 20% to 40%, or by 30%, e.g., relative to a control AAV vector or relative to a cell in the hippocampus of the subject that is not transduced. In some embodiments, the level of GluK2 is reduced for at least 28 days, at least 30 days, at least 60 days, at least 90 days, at least 120 days, at least 150 days, at least 180 days, or at least 365 days, at least 2 years, 3 years, 4 years, 5 years, 10 years, 15 years, or 20 years, or for the life of the subject.

In some embodiments of any of the foregoing aspects, the vector (e.g., the AAV vector) is administered to the subject in an amount of about 1×10¹¹ vg/mL, 2×10¹¹ vg/mL, 3×10¹¹ vg/mL, 4×10¹¹ vg/mL, 5×10¹¹ vg/mL, 6×10¹¹ vg/mL, 7×10¹¹ vg/mL, 8×10¹¹ vg/mL, 9×10¹¹ vg/mL, 1×10¹² vg/mL, 2×10¹² vg/mL, 3×10¹¹ vg/mL, 4×10¹² vg/mL, 5×10¹² vg/mL, 6×10¹² vg/mL, 7×10¹² vg/mL, 8×10¹² vg/mL, 9×10¹² vg/mL, or 1×10¹³ vg/mL. In some embodiments, the vector (e.g., the AAV vector) is administered to the subject in an amount of from about 3×10⁸ vg/mm³ hippocampus to about 1.2×10⁹ vg/mm³ hippocampus. In some embodiments, the vector (e.g., the AAV vector) is formulated to be administered to the subject in an amount of from about 9×10¹¹ total vg/hippocampus to about 3.6×10¹² total vg/hippocampus.

In some embodiments of any of the foregoing aspects, the composition is formulated to provide the vector (e.g., the AAV vector) to the subject in a single dose per hemisphere including the amount, and, for example, the composition containing the vector (e.g., the AAV vector) is administered by advancing a needle through the brain (e.g., the hippocampus) and delivering a volume of the composition at between 1-10, between 2-9, between 3-8, between 4-7, or between 5-6 focal sites within the brain (e.g., the hippocampus), in which the total volume of the single dose is divided by the number of focal sites. Optionally, the focal sites are determined or monitored by an MRI or PET scan. In some embodiments, the composition is formulated to provide the vector (e.g., the AAV vector) to the subject in a single dose per hemisphere at five or fewer focal sites within the brain (e.g., the hippocampus), in which the volume of the single dose is divided by the number of focal sites. In some embodiments, the vector (e.g., the AAV vector) is administered to the subject in a volume of about 0.5 mL to 1.8 mL, about 0.75 mL to 1.5 mL, about 1 mL to 1.25 mL, or about 1.15 mL.

In some embodiments of any of the foregoing aspects, the ribopolynucleotide includes a nucleic acid sequence that encodes miR3bR (e.g., SEQ ID NO: 14, or a variant thereof with at least 95% sequence identity thereto), miR38R (e.g., SEQ ID NO: 15, or a variant thereof with at least 95% sequence identity thereto), or both miR3bR and miR38R (e.g., SEQ ID NO: 13 or 16, or a variant thereof with at least 95% sequence identity thereto). In an embodiment, administration of the composition results in expression of 1×10³ to 1×10⁷ copies/nanogram of RNA of both miR3bR and miR38R (e.g., SEQ ID NO: 13 or 16, or a variant thereof with at least 95% sequence identity thereto) in a cell of the subject. In some embodiments, the subject expresses about 1×10⁵ copies/nanogram of RNA of both miR3bR and miR38R (e.g., SEQ ID NO: 13 or 16, or a variant thereof with at least 95% sequence identity thereto).

In some embodiments of any of the foregoing aspects, the vector (e.g., the AAV vector) is expressed in a cell of the brain, for example a cell of the hippocampus of the subject, wherein the cell is a hippocampal neuron (e.g., a dentate granule cell (DGC) or a glutamatergic pyramidal neuron). In some embodiments, expression of the vector (e.g., the AAV vector) does not occur in peripheral tissues (e.g., liver or heart) of the subject or occurs at a level of 10% or less relative to expression in, e.g., a transduced neuron. In some embodiments, expression of the vector (e.g., the AAV vector) in the subject's DRG, blood, and/or CSF occurs at a level of 10% or less relative to expression in, e.g., a transduced neuron. In some embodiments, expression of the vector (e.g., the AAV vector) in the subject's DRG, blood, and/or CSF occurs at a level of 1×10⁶ ds vg/μg of DNA or ds vg/mL biofluid or less (e.g., 1×10⁵ ds vg/μg of DNA or ds vg/mL biofluid, 1×10⁴ ds vg/μg of DNA or ds vg/mL biofluid, 1×10³ ds vg/μg of DNA or ds vg/mL biofluid, 1×10² ds vg/μg of DNA or ds vg/mL biofluid, 10 ds vg/μg of DNA or ds vg/mL biofluid, or less). In some embodiments, expression of the vector (e.g., the AAV vector) in the subject's DRG, blood, and/or CSF occurs at a level of 1×10⁵ ds vg/μg of DNA or ds vg/mL biofluid or less. In some embodiments, expression of the vector (e.g., the AAV vector) in the subject's DRG, blood, and/or CSF occurs at a level of 1×10⁴ ds vg/μg of DNA or ds vg/mL biofluid or less. In some embodiments, expression of the vector (e.g., the AAV vector) in the subject's DRG, blood, and/or CSF occurs at a level of 1×10³ ds vg/μg of DNA or ds vg/mL biofluid or less. In some embodiments, expression of the vector (e.g., the AAV vector) in the subject's DRG, blood, and/or CSF occurs at a level of 1×10² ds vg/μg of DNA or ds vg/mL biofluid or less. In some embodiments, expression of the vector (e.g., the AAV vector) in the subject's DRG, blood, and/or CSF occurs at a level of 10 ds vg/μg of DNA or ds vg/mL biofluid or less.

In some embodiments of any of the foregoing aspects, the composition:
(a) reduces the number of seizures per day and/or reduces epileptiform discharges in the subject, for example, as measured by an electroencephalogram and standardized to seizure frequency per 30 days;
(b) improves the subject's measurements on routine laboratory parameters, such as hematology, biochemistry, coagulation, and urinalysis parameters, within at least 4 weeks, at least 5 weeks, at least 6 weeks, at least 7 weeks, at least 8 weeks, at least 3 months, at least 4 months, at least 5 months, at least 6 months, or at least 1 year after administration of the composition;
(c) reduces frequency of interictal discharges, as measured by an electroencephalogram;
(d) reduces aberrant neurological behavior by the subject; and
(e) produces no adverse effects after 4 weeks or 8 weeks.

In some embodiments of any of the foregoing aspects, the vector (e.g., the AAV vector) is for administration once per year (e.g., once per 2, 3, 4, 5, 6, 7, 8, 9, 10, 15, 20, 25, or 30 years, or more (e.g., 100 years or once in the life of the subject)). In some embodiments, the vector (e.g., the AAV vector) is administered to the subject once in their lifetime.

In some embodiments of any one of the foregoing aspects, the method includes administering the composition of any one of the foregoing aspects to the subject.

### Definitions

Unless stated otherwise, or implicit from context, the following terms and phrases include the meanings provided below. The definitions are provided to aid in describing particular embodiments and are not intended to limit the claimed technology. Unless otherwise defined, all technical and scientific terms used herein have the same meaning as commonly understood by one of ordinary skill in the art to which this technology belongs.

In this application, unless otherwise clear from context, (i) the term “a” may be understood to mean “at least one”; (ii) the term “or” may be understood to mean “and/or”; and (iii) the terms “including” and “comprising” may be understood to encompass itemized components or steps whether presented by themselves or together with one or more additional components or steps.

The term “about” refers to an amount that is ±10% of the recited value and may be ±5% of the recited value or ±2% of the recited value.

The term “administration” refers to providing or giving a subject a therapeutic agent (e.g., an inhibitory ribopolynucleotide that binds to and inhibits the expression of a GRIK2 mRNA, or a vector encoding the same, as is disclosed herein), by any effective route. Exemplary routes of administration are described herein and below (e.g., intracerebroventricular injection, intrathecal injection, intraparenchymal injection, intravenous injection, and stereotactic injection).

The term “adeno-associated viral vector” or “AAV vector” refers to a vector derived from an adeno-associated virus serotype, including without limitation, AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV-TT, AAV-DJ8, or AAV.HSC16. AAV vectors can have one or more of the AAV wild-type genes deleted in whole or part, e.g., the rep and/or cap genes, but retain functional flanking ITR sequences. Functional ITR sequences promote the rescue, replication, and packaging of the AAV virion. Thus, an AAV vector is defined herein to include at least those sequences required in cis for replication and packaging (e.g., functional ITRs) of the virus. ITRs do not need to be the wild-type polynucleotide sequences and may be altered, e.g., by the insertion, deletion, or substitution of nucleotides, so long as the sequences provide for functional rescue, replication, and packaging. AAV expression vectors are constructed using known techniques to at least provide as operatively linked components in the direction of transcription, control elements including a transcriptional initiation region, the DNA of interest (e.g., a polynucleotide encoding an inhibitory RNA agent of the disclosure) and a transcriptional termination region.

The terms “antisense oligonucleotide” and “ASO” refer to an inhibitory polynucleotide capable of hybridizing through complementary base-pairing with a target mRNA molecule (e.g., a GRIK2mRNA) and inhibiting its expression through mRNA destabilization and degradation, or inhibition of translation.

The terms “disrupt expression of,” “inhibit expression of,” or “reduce the expression of,” with respect to a gene (e.g., GRIK2), refers to preventing, reducing, or inhibiting the formation of a functional gene product (e.g., a GluK2 protein). A gene product is functional if it fulfills its normal (wild-type) function(s). Disruption of the gene prevents or reduces the expression of a functional protein encoded by the gene. The disrupted gene may be disrupted by, e.g., an interfering RNA molecule (e.g., an ASO), such as those described herein.

The terms “effective amount” and “sufficient amount” applied to, e.g., a composition, polyribonucleotide, or vector described herein refer to a quantity that, when administered to the subject, including a mammal, for example a human, achieves beneficial or desired results, including clinical results in the subject. For example, in the context of treating temporal lobe epilepsy (TLE), an “effective amount” or synonym thereof can be considered an amount of the composition, polyribonucleotide or vector that achieves a treatment response (e.g., a reduction in one or more symptoms of TLE, such as a reduction in seizure activity, as defined here), as compared to the response obtained without administration of the composition, polyribonucleotide, or vector.

The term “epilepsy” refers to one or more neurological disorders that clinically present with recurrent epileptic seizures. Epilepsy can be classified according the electroclinical syndromes following the Classification and Terminology of the International League Against Epilepsy (ILAE; Berg et al., 2010). These syndromes can be categorized by age at onset, distinctive constellations (surgical syndromes), and structural-metabolic causes, such as: (A) age at onset: (i) neonatal period includes benign familial neonatal epilepsy (BFNE), early myoclonic encephalopathy (EME), Ohtahara syndrome; (ii) infancy period includes epilepsy of infancy with migrating focal seizures, West syndrome, myoclonic epilepsy in infancy (MEI), benign infantile epilepsy, benign familial infantile epilepsy, Dravet syndrome, myoclonic encephalopathy in nonprogressive disorders; (iii) childhood period includes febrile seizures plus (FS+), Panayiotopoulos syndrome, epilepsy with myoclonic atonic (previously astatic) seizures, benign epilepsy with centrotemporal spikes (BECTS), autosomal-dominant nocturnal frontal lobe epilepsy (ADNFLE), late onset childhood occipital epilepsy (Gastaut type), epilepsy with myoclonic absences, Lennox-Gastaut syndrome, epileptic encephalopathy with continuous spike-and-wave during sleep (CSWS), Landau-Kleffner syndrome (LKS), childhood absence epilepsy (CAE); (iv) adolescence-adult period includes juvenile absence epilepsy (JAE) juvenile myoclonic epilepsy (JME), epilepsy with generalized tonic-clonic seizures alone, progressive myoclonus epilepsies (PME), autosomal dominant epilepsy with auditory features (ADEAF), other familial temporal lobe epilepsies; (v) variable age onset includes familial focal epilepsy with variable foci (childhood to adult), reflex epilepsies; (B) distinctive constellations (surgical syndromes) include mesial temporal lobe epilepsy (MTLE), Rasmussen syndrome, gelastic seizures with hypothalamic hamartoma, hemiconvulsion-hemiplegia-epilepsy; (C) epilepsies attributed to and organized by structural-metabolic causes include malformations of cortical development (hemimegalencephaly, heterotopias, etc.), neurocutaneous syndromes (tuberous sclerosis complex and Sturge-Weber), tumor, infection, trauma, angioma, perinatal insults, and stroke. The term “refractory epilepsy” refers to an epilepsy which is refractory to pharmaceutical treatment (e.g., treatment with an anti-epileptic drug); that is to say that current pharmaceutical treatment does not allow an effective treatment of a patient's disease (see, for example, Englot et al. (Journal of Neurosurgery. 118(1): 169-74. 2013)). Non-limiting examples of anti-epileptic drugs include narrow-spectrum anti-epileptic drugs and broad-spectrum anti-epileptic drugs. Narrow-spectrum anti-epileptic drugs are primarily used to treat focal seizures and include Carbamazepine (Carbatrol, Tegretol, Epitol, Equetro), Eslicarbazepine (Aptiom) Everolimus (Afinitor, Afinitor Disperz), Gabapentin (Neurontin), Lacosamide (Vimpat), Oxcarbazepine (Trileptal, Oxtellar XR), Phenobarbital, Phenytoin (Dilantin, Phenytek), Pregabalin (Lyrica), Tiagabine (Gabitril), and Vigabatrin (Sabril). Broad-spectrum anti-epileptic drugs include Brivaracetam (Briviact), Cannabidiol (Epidiolex), Cenobamate (Xcopri), Clorazepate (Gen-Xene, Tranxene-T), Divalproex (Depakote, Depakote ER), Felbamate (Felbatol), Lamotrigine (Lamictal, Lamictal CD, Lamictal ODT, Lamictal XR), Levetiracetam (Elepsia XR, Keppra, Keppra XR, Spritam), Lorazepam (Ativan), Perampanel (Fycompa), Primidone (Mysoline), Rufinamide (Banzel), Topiramate (Topamax, Qudexy XR, Trokendi XR), Valproic acid, and Zonisamide (Zonegran).

The term “expression” when used in the context of expression of a gene or nucleic acid refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include mRNAs, which are modified by processes such as capping, polyadenylation, methylation, and editing, and proteins (e.g., GluK2) modified by, for example, methylation, acetylation, phosphorylation, ubiquitination, SUMOylation, ADP-ribosylation, myristoylation, and glycosylation.

The term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. Expression of a gene of interest in a subject can manifest, for example, by detecting: a decrease or increase in the quantity or concentration of mRNA encoding a corresponding protein (as assessed, e.g., using RNA detection procedures described herein or known in the art, such as quantitative polymerase chain reaction (qPCR) and RNA seq techniques), a decrease or increase in the quantity or concentration of a corresponding protein (as assessed, e.g., using protein detection methods described herein or known in the art, such as enzyme-linked immunosorbent assays (ELISA), among others), and/or a decrease or increase in the activity of a corresponding protein (e.g., in the case of an ion channel, as assessed using electrophysiological methods described herein or known in the art) in a sample obtained from the subject.

The term “GluK2”, also known as “GluR6”, “GRIK2”, “MRT6”, “EAA4”, or “GluK6”, refers to the glutamate ionotropic receptor kainate type subunit 2 protein (XP_047274637.1, NP_068775.1, XP_047274638.1, XP_016866270.1, XP_016866271.1, NP_786944.1, NP_001159719.1, XP_005267003.1) as named in the currently used IUPHAR nomenclature (Collingridge, G.L., Olsen, R.W., Peters, J., Spedding, M., 2009. A nomenclature for ligand-gated ion channels. Neuropharmacology 56, 2-5). The terms “GluK2-containing KAR,” “GluK2 receptor,” “GluK2 protein,” and “GluK2 subunit” may be used interchangeably throughout and generally refer to the protein encoded by or expressed by a GRIK2 gene.

The term “ionotropic glutamate receptors” include members of the NMDA (N-methyl-D-aspartate), AMPA (α-amino-3-hydroxy-5-methyl-4-isoxazoleproprionic acid) and kainate receptor (KAR) classes. Functional KARs can be assembled into tetrameric assemblies from the homomeric or heteromeric combination of five subunits named GluK1, GluK2, GluK3, GluK4 and GluK5 subunits (Reiner et al., 2012). The targets of the disclosure are, in some instances, KAR complexes composed of GluK2 and GluK5. Inhibiting the expression of GRIK2gene is sufficient to abolish GluK2/GluK5 kainate receptor function, given the observation that the GluK5 subunit by itself does not form functional homomeric channels.

An “inhibitor of expression” refers to an agent (e.g., an inhibitory RNA agent (e.g., an inhibitory ribopolynucleotide) of the disclosure) that has a biological effect to inhibit or decrease the expression of a gene, e.g., the GRIK2 gene (NC_000006.12:101393708-102070083, XM_047418681.1, NM_021956.5, XM_047418682.1, XM_017010781.3, XM_017010782.3, NM_175768.3, NM_001166247.1, XM_005266946.5). Inhibiting expression of a gene, e.g., the GRIK2gene, will typically result in a decrease or even abolition of expression of the gene product (protein, e.g., GluK2 protein) in target cells or tissues, although various levels of inhibition may be achieved. Inhibiting or decreasing expression is typically referred to as knockdown.

The term “isolated polynucleotide” refers to an isolated molecule including two or more covalently linked nucleotides. Such covalently linked nucleotides may also be referred to as nucleic acid molecules. Generally, an “isolated” polynucleotide refers to a polynucleotide that is man-made, chemically synthesized, purified, and/or heterologous with respect to the nucleic acid sequence from which it is obtained.

The term “microRNA” refers to a short (e.g., typically ˜22 nucleotide) sequence of non-coding RNA that regulates mRNA translation and thus influences target protein abundance. Some microRNAs are transcribed from a single, monocistronic gene, while others are transcribed as part of polycistronic gene clusters. The structure of a microRNA may include 5′ and 3′ flanking sequences, hairpin sequences including stem and loop sequences. During processing within the cell, an immature microRNA is truncated by Drosha, which cleaves off the 5′ and 3′ flanking sequences. Subsequently, the microRNA molecule is translocated from the nucleus to the cytoplasm, where it undergoes cleavage of the loop region by Dicer. The biological action of microRNAs is exerted at the level of translational regulation through binding to regions of the mRNA molecule, typically the 3′ untranslated region, and leading to the cleavage, degradation, destabilization, and/or less efficient translation of the mRNA. Binding of the microRNA to its target is generally mediated by a short (e.g., 6-8 nucleotide) “seed region/sequence” within the hairpin sequence of the microRNA. Throughout the disclosure, the term siRNA may include its equivalent miRNA, such that the miRNA encompasses the same bases that have homology to the target (e.g., in the seed region) as its equivalent siRNA. As described herein, a microRNA may be a non-naturally occurring microRNA, such as a microRNA having one or more heterologous nucleic acid sequences.

The term “nucleotide” is defined as a modified or naturally occurring deoxyribonucleotide or ribonucleotide. Nucleotides typically include purines and pyrimidines, which include thymidine, cytidine, guanosine, adenosine and uridine. The term “inhibitory polynucleotide” as used herein is defined as an oligomer of the nucleotides defined above or modified nucleotides disclosed herein. The term “inhibitory polynucleotide” refers to a nucleic acid sequence, 3′-5′ or 5′-3′ oriented, which may be single- or double-stranded. The inhibitory polynucleotide used in the context of the disclosure may in particular be DNA or RNA. The term may also include an “inhibitory polynucleotide analog,” which refers to an inhibitory polynucleotide having, e.g., (i) a modified backbone structure, e.g., a backbone other than the standard phosphodiester linkage found in natural oligo- and polynucleotides, and (ii) optionally, modified sugar moieties, e.g., morpholino moieties rather than ribose or deoxyribose moieties. Inhibitory polynucleotide analogs support bases capable of hydrogen bonding by Watson-Crick base pairing to standard polynucleotide bases, where the analog backbone presents the bases in a manner to permit such hydrogen bonding in a sequence-specific fashion between the inhibitory polynucleotide analog molecule and bases in a standard polynucleotide {e.g., single-stranded RNA or single-stranded DNA). Particularly, analogs are those having a substantially uncharged, phosphorus containing backbone. A substantially uncharged, phosphorus containing backbone in an inhibitory polynucleotide analog is one in which a majority of the subunit linkages, e.g., between 50-100%, typically at least 60% to 100% or 75% or 80% of its linkages, are uncharged, and contain a single phosphorous atom. Furthermore, the term “inhibitory polynucleotide” can include an inhibitory polynucleotide sequence that is inverted relative to its normal orientation for transcription and so corresponds to an RNA or DNA sequence that is complementary to a target gene mRNA molecule expressed within the host cell. An antisense guide strand may be constructed in a number of different ways, provided that it is capable of interfering with the expression of a target gene. For example, the antisense guide strand can be constructed by reverse-complementing the coding region (or a portion thereof) of the target gene relative to its normal orientation for transcription to allow the transcription of its complement, (e.g., RNAs encoded by the antisense and sense gene may be complementary). The inhibitory polynucleotide need not have the same intron or exon pattern as the target gene, and noncoding segments of the target gene may be equally effective in achieving antisense suppression of target gene expression as coding segments such as an ASO. In some cases, the inhibitory RNA has the same exon pattern as the target gene.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are well-known in the art, for example, using publicly available computer software such as BLAST, BLAST-2, or Megalign software. Using well-recognized and conventional methods, the appropriate parameters can be determined for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

$$100 multiplied by ( the fraction X / Y )$$

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A. Regardless of the percent sequence identity between a candidate sequence and a reference polynucleotide or polypeptide sequence, the candidate sequence retains at least 20%, 30%, 40%, 50%, 60%, 70%, 75%, 80%, 85%, 90%, 95%, 97%, 99%, or 100% of the function (e.g., the ability to reduce a level of GRIK2 mRNA, as defined herein, or a level of expression of GluK2 protein, as defined herein) of the reference polynucleotide or polypeptide sequence.

The term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

The term “pharmaceutical composition,” as used herein, represents a composition containing a compound (e.g., an inhibitory nucleic acid molecule (e.g., a ribopolynucleotide, such as an RNA) or vector containing the same) described herein formulated with a pharmaceutically acceptable excipient, and in some instances may be manufactured or sold with the approval of a governmental regulatory agency as part of a therapeutic regimen for the treatment of disease in a mammal. Pharmaceutical compositions can be formulated, for example, for parenteral administration, such as intravenous administration (e.g., as a sterile solution free of particulate emboli and in a solvent system suitable for intravenous use), intrathecal injection, intracerebroventricular injections, intraparenchymal injection, or in any other pharmaceutically acceptable formulation.

The terms “target” or “targeting” refers to the ability of an inhibitory nucleic acid molecule (e.g., a ribopolynucleotide, such as an RNA), such as an inhibitory RNA agent described herein, to specifically bind through complementary base pairing to a GRIK2gene or mRNA encoding a GluK2 protein.

The terms “short interfering RNA” and “siRNA” refer to an inhibitory polynucleotide containing double stranded nucleic acid in which each strand comprises RNA, RNA analog(s) or RNA and DNA. The siRNA molecule can include between 19 and 23 nucleotides (e.g., 21 nucleotides). The siRNA typically has 2 bp overhangs on the 3′ ends of each strand such that the duplex region in the siRNA comprises 17-21 nucleotides (e.g., 19 nucleotides). Typically, the antisense strand of the siRNA is sufficiently complementary with the target sequence of the target gene/RNA. siRNA molecules operate within the RNA interference pathway, leading to inhibition of mRNA expression by binding to a target mRNA (e.g., GRIK2 mRNA) and degrading the mRNA through Dicer-mediated mRNA cleavage. Throughout the disclosure, the term siRNA is meant to include its equivalent miRNA, such that the miRNA encompasses the same bases that have homology to the target as its equivalent siRNA.

The terms “short hairpin RNA” and “shRNA” refer to an inhibitory polynucleotide containing single-stranded RNA of 50 to 100 nucleotides that forms a stem-loop structure in a cell, which contains a loop region of 5 to 30 nucleotides, and long complementary RNAs of 15 to 50 nucleotides at both sides of the loop region, which form a double-stranded stem by base pairing between the complementary RNA sequences; and, in some cases, an additional 1 to 500 nucleotides included before and after each complementary strand forming the stem. For example, shRNA generally requires specific sequences 3′ of the hairpin to terminate transcription by RNA polymerase. Such shRNAs generally bypass processing by Drosha due to their inclusion of short 5′ and 3′ flanking sequences. Other shRNAs, such as “shRNA-like microRNAs,” which are transcribed from RNA polymerase II, include longer 5′ and 3′ flanking sequences, and require processing in the nucleus by Drosha, after which the cleaved shRNA is exported from the nucleus to cytosol and further cleaved in the cytosol by Dicer. Like siRNA, shRNA binds to the target mRNA in a sequence specific manner, thereby cleaving and destroying the target mRNA, and thus suppressing expression of the target mRNA.

The terms “subject” and “patient” refer to an animal (e.g., a mammal, such as a human). A subject to be treated according to the methods described herein may be one who has been diagnosed with an epilepsy (e.g., TLE or focal epilepsy), or one who exhibits one or more symptoms of epilepsy (e.g., seizures). Diagnosis may be performed by any method or technique known in the art. A subject to be treated according to the disclosure may have been subjected to standard tests or may have been identified as a candidate for treatment based on their symptoms. For example, the subject:
(a) may have or experience:
   (i) at least 12 documented seizures during the previous 90 days, in which the seizures include, e.g.:
      at least 2 documented focal impaired awareness seizures; and/or
      at least 10 documented focal aware seizures;
   (ii) no 21-day seizure-free period in the previous 90 days;
   (iii) hippocampal atrophy, for example, as determined by MRI-T1 imaging, optionally with:
      increased ipsilateral mesial signal on T2 imaging; or
      ipsilateral hypometabolism on fluorodeoxyglucose positron emission tomography (FDG-PET);
   (iv) a score of 23 or above on a Montreal Cognitive Assessment (MoCA); and/or
   (v) no significant focal neurocognitive dysfunction, inconsistent with disease pathology-related magnetic resonance imaging (MRI) and positron emission tomography (PET) imaging findings; and/or
(b) may not have:
   (i) lesions on neuroimaging outside of the mesial temporal love area, temporal neocortical or extratemporal lesions on MRI, or diffuse unilateral or bilateral hypometabolism on PET;
   (ii) a progressive neurological disorder;
   (iii) a psychogenic seizure within the last 2 years;
   (iv) an implanted device that would contraindicate MRI-guided convection-enhanced delivery (CED), such as a vagus nerve stimulation (VNS) device or a cochlear implant;
   (vi) a major disease-unrelated neurosurgical intervention due to intracranial tumor, trauma, or bleeding; and/or
   (vii) a medical history of schizophrenia.

The terms “temporal lobe epilepsy” or “TLE” refers to a chronic neurological condition characterized by chronic and recurrent seizures (epilepsy) which originate in the temporal lobe of the brain. This disease is different from acute seizures in naïve brain tissue since TLE is characterized by morpho-functional reorganization of neuronal networks and sprouting of recurrent mossy fibers from granule cells of the dentate gyrus of the hippocampus, whereas acute seizures in naïve tissue do not precipitate such circuit-specific reorganization. TLE may result from an emergence of an epileptogenic focus in one or both hemispheres of the brain.

The terms “transduction” and “transduce” refer to a method of introducing a nucleic acid material (e.g., a vector, such as a viral vector construct, or a part thereof) into a cell and subsequent expression of a polynucleotide encoded by the nucleic acid material (e.g., the vector construct or part thereof) in the cell.

The term “treatment” or “treat” refers to both prophylactic and preventive treatment as well as curative or disease modifying treatment, including treatment of a human subject displaying or diagnosed as having one or more symptoms of epilepsy, such as a human subject experiencing seizures. The treatment may be administered to a subject having a medical disorder or displaying one or more symptoms of such a disorder, in order to reduce the severity of, or ameliorate one or more symptoms of the disorder beyond that expected in the absence of such treatment. By “therapeutic regimen” is meant the pattern of treatment of an illness, e.g., the pattern of dosing used during therapy.

The term “vector” includes a nucleic acid vector, e.g., a DNA vector, such as a plasmid, an RNA vector, or another suitable replicon (e.g., viral vector). A variety of vectors have been developed for the delivery of polynucleotides encoding exogenous polynucleotides or proteins into a eukaryotic cell. Examples of such expression vectors are disclosed in, e.g., WO 1994/011026; incorporated herein by reference as it pertains to vectors suitable for the expression of a nucleic acid material of interest. Expression vectors suitable for use with the compositions and methods described herein contain a polynucleotide sequence as well as, e.g., additional sequence elements used for the expression of heterologous nucleic acid materials (e.g., an ASO) in a mammalian cell. Certain vectors that can be used for the expression of the inhibitory nucleic acid (e.g., a ribopolynucleotide, such as an RNA) agents described herein include plasmids that contain regulatory sequences, such as promoter and enhancer regions, which direct gene transcription. Other useful vectors for expression of inhibitory nucleic acid (e.g., RNA) agents disclosed herein contain polynucleotide sequences that enhance the rate of translation of these polynucleotides or improve the stability or nuclear export of the nucleic acid (e.g., RNA) that results from gene transcription. These sequence elements include, e.g., 5′ and 3′ untranslated regions, an IRES, and polyadenylation signal sequence site in order to direct efficient transcription of the gene carried on the expression vector.

As used herein, the term “variant” refers to a polynucleotide, such as, e.g., an inhibitory polynucleotide sequence of the disclosure or a complement thereof (e.g., substantial or full complement thereof) which is obtained by rationally including one or more (e.g., 1, 2, 3, 4, 5, 6, or 7) nucleotide modifications (substitutions, insertions, deletions, or mismatches) to a starting sequence (e.g., a reference sequence). Such modifications may improve at least one characteristic (e.g., a biological function) of the polynucleotide (e.g., improved RISC loading or retention of a guide strand, reduced RISC loading or retention of the passenger strand, or increased ratio of guide-to-strand production, and improved inhibition of a target nucleic acid sequence).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a graph showing fold change of GluK2 protein expression after transduction of different constructs in mouse cortical neurons. Data are represented as mean+/−SEM. ANOVA followed by Dunnett's multiple comparisons test, test versus control, *P<0.05, **P<0.01, and ****P<0.0001. (Construct D (GFP control): hSyn.GFP, NT: not transfected, Construct A (null control): Control with no coding sequence, Construct E: hSyn.miR30.amiR38; Construct F: hSyn.amiR38v2.E-miR-30.amiR3b.E-miR-218-1, Construct B: hSyn.amiR38v2R3.E-miR-30.amiR3bR2.E-miR-218-1, Construct G: hSyn.amiR38v2R3.E-miR-30.stuffer, Construct H: hSyn.amiR3bR2.E-miR-218-1.stuffer).

FIG. 2 is a graph showing quantification of epileptiform discharges from human organotypic slices activity after treatment with Construct B at 1×10¹⁰ vg/slice or control (Construct D (GFP control), Construct A (null control), or Construct M) at 1.8×10¹⁰ vg/slice. Epileptiform activity is captured as percent change of the epileptic discharge frequency compared to the baseline condition (treatment of the slices with 4-EPI-Gabosine). Data are represented as mean+/−SEM. *P<0.05, t-test. Results obtained from 7 different donors showed that treatment with Construct B leads to significant decrease in epileptiform discharges when compared to controls.

FIG. 3 is a graph showing the distance traveled in the open field test by pilocarpine-treated mice before and after injection of Construct B (double construct), Construct G and Construct H (single constructs), or the control vector (Construct A, null control). The dotted line represents the average level of activity of 20 naïve wild-type mice. Results are presented as mean±SD. ****P<0.0001, two-way ANOVA repeated measure followed by Sidak's multiple comparison test. Note that not all construct used in RES-2021-020 study are included in this analysis. Lines represent changes in individual animals pre- and post-injection.

FIGS. 4A-4C are graphs showing (FIG. 4A) vector genome, (FIG. 4B), miR38R, and (FIG. 4C) miR3bR quantification in hippocampi of pilocarpine-treated mice injected with Construct A (null control) at 1×10¹⁰ vg/brain as a control or Construct B at 1×10⁸, 1×10⁹, and 1×10¹⁰ vg/brain. Results are presented as mean+/−SEM.

FIG. 5 is a graph showing the distance traveled by pilocarpine-treated mice before and after injection of Construct B or injection of the control AAV9-vector (Construct A, null control). He dotted line represents the average level of activity of 20 naïve wild-type mice. Results are presented as mean±SEM.***P<0.0001, two-way ANOVA repeated measure followed by Sidak's multiple comparison test. ** P<0.0001, two-way ANOVA repeated measure followed by Sidak's multiple comparison test. Lines represent changes in individual animals pre- and post-injection.

FIG. 6 is a graph showing the number of seizures per day in pilocarpine-treated mice after injection of AAV-constructs expressing Construct B or injection of a control vector (Construct A, null control). Results are presented as mean+/−SD. *P<0.05, student t-test.

FIG. 7 is an image of an axial reconstruction of planned laser ablation trajectory (red) with an outline of the hippocampus (yellow) and amygdala (cyan). This figure is FIG. 3 reproduced from Vakharia et al. (Annals of Neurology. 83 (4):676-690, 2018).

FIG. 8 shows four hippocampal images of cynomolgus monkeys taken at different angles to illustrate top-down administration conduction of Construct B showing good spread of the gadolinium formulation throughout the hippocampus.

FIG. 9 is a graph showing reduction of GluK2 expression after transduction of different constructs. Data are represented as mean±SEM. ANOVA followed by Dunnett's multiple comparisons test, test versus control, **P<0.01 and ****P<0.0001.

FIGS. 10A and 10B show results from administration of Construct D (GFP control) or Construct B in ex-vivo human brain organotypic slices. FIG. 10A shows a representative electroencephalogram (EEG) trace obtained with resected TLE patient organotypic slices. FIG. 10B is a graph showing quantification of epileptiform discharges after transduction with Construct D or Construct B at 1×10¹⁰ vg/slice.

FIG. 11 is a graph showing vector genome (vg) quantification in the left and right hippocampi of pilocarpine-treated mice injected with Construct A (null control, left) as a control or Construct B (right). Results are presented as mean+/−SEM.

FIG. 12 is a graph showing distance traveled by pilocarpine-treated mice before and after injection of Construct A (left two bars) as a control or Construct B (right two bars). Results are presented as mean±SEM. The dotted line represents the average level of activity of 20 naïve wild-type mice. *P<0.05, **P<0.0001, two-way ANOVA repeated measure followed by Sidak's multiple comparison test. Lines represent changes in individual animals pre- and post-injection.

FIGS. 13A and 13B are a set of photomicrographs showing the comparison of expression from AAV9.hsyn.GFP (FIG. 13A; 2×10¹⁰ dose) and AAV.CAG.GFP (FIG. 13B; 1×10⁹ dose). The arrows indicate area where there is a lack of neuronal expression in the CA2 layer and the hillus/polymorphic region of the dentate gyrus (DG) with AAV.CAG.GFP.

FIG. 14 is a set of photomicrographs showing high magnification images of GFP expression in the DG following AAV.hsyn.GFP administration as shown in FIG. 13. GFP expression is restricted to neurons following administration of the vector.

FIG. 15 is a set of photomicrographs showing MRI-guided intra-hippocampal delivery of 60 μl of Construct D by CED using a CLEARPOINT® Neuro SMARTFLOW® cannula using top-down delivery through the top of the skull of a pilocarpine mouse. PROHANCE®, a gadolinium-based contrast agent (gadoteridol) was co-infused at a concentration of 2 mM to monitor the infusate distribution. Image shows start of infusion (left panel), mid-infusion (middle panel) and end-of-infusion (right panel).

FIG. 16 is an image showing eGFP immunostaining of the hippocampus in a pilocarpine mouse at low magnification.

FIG. 17 is an image showing eGFP immunostaining of the same hippocampus in the pilocarpine mouse shown in FIG. 20 at high magnification.

FIG. 18 is an image showing eGFP immunostaining of the same hippocampus in the pilocarpine mouse shown in FIGS. 20 and 21 at high magnification.

FIG. 19 is a graph showing the biodistribution of AAV9 vector genome by quantitative real-time PCR (qPCR) in brain regions. The highest levels of AAV9 vector genome were detected in the left hippocampus. Levels in other parts of the brain, spinal cord, and DRG were 100-1000 fold lower than those in the left hippocampus, except for the entorhinal cortex.

FIG. 20 is a graph showing the biodistribution of AAV9 vector genome by qPCR in peripheral organs. Levels of AAV9 in peripheral organs (liver, lung, heart, and kidney) were negligible, but higher in the spleen.

FIGS. 21A and 21B are pictographic representation of the nucleotide sequence alignment between the human (hsa), cynomolgus monkey (mcf), and mouse (mmu) mRNA sequences at the mature miR sites (FIG. 21A, miR-38 (SEQ ID NOs: 18-21; FIG. 21B, miR-3b (SEQ ID NOs: 22-25)) showing perfect complementarity in the key pairing regions for human, monkey, and mouse.

FIGS. 22A-22F are images showing a brain punch map for the cynomolgus monkey. Twelve (6 male, 6 female) cynomolgus monkeys received bilateral infusions of diluent or Construct B into the hippocampus, delivered by CED using a CLEARPOINT® Neuro SMARTFLOW® cannula. PROHANCE®, a gadolinium-based contrast agent (gadoteridol) was co-infused at a concentration of 2 mM to monitor the infusate distribution. Dose administration was guided by real-time MRI using the same trajectory and procedure as with Construct D (GFP control). The volume remained the same for all injections. The dose was administered at a rate of 1 to 3 μL/minute. The high dose of 2×10¹³ vg/mL was approaching the maximum feasible dose based on the volume administered and the vector titer of the stock (5.15×10¹³ vg/mL). The mid- and low-dose levels were selected to be 10-fold and a 100-fold lower vg/mL concentration than the high-dose level. Blood and CSF were collected for immunogenicity and biodistribution assessment. On Day 29±2, the animals were sacrificed, and selected tissues were harvested for histopathological and biodistribution evaluation. The brain was sliced coronally at 3 to 4 mm slice thickness. 4 mm brain punches (FIG. 22A, slice 7; FIG. 22B, slice 8; FIG. 22C, slice 9; FIG. 22D, slice 10; FIG. 22E, slice 11; FIG. 22F, slice 13) were collected and analyzed for AAV9 vector genome, mature miRNA expression, and GRIK2 mRNA levels.

FIG. 23 is a graph showing the group mean Construct B vector genome (vg) biodistribution by qPCR assay in the hippocampus and other brain punches, the spinal cord, the DRG, and the peripheral organs.

FIG. 24 is a graph showing the group mean Construct B vector genome biodistribution in the blood and CSF, shown as double stranded vector genome (vg) per μg of host DNA or mL CSF.

FIGS. 25A-25D are graphs showing the correlation of Construct B double stranded vector genome (vg) per μg of host DNA versus miR3bR in individual hippocampal brain punches of high-dose cynomolgus monkeys (FIG. 25A, adult male subject 1; FIG. 25B, adult male subject 2; FIG. 25C, adult female subject 1; FIG. 25D, adult female subject 2).

FIG. 26 is a graph showing miR38R expression in various tissues and CSF.

FIG. 27 is a graph showing miR3bR expression in various tissues and CSF.

FIGS. 28A-28D is a series of graphs showing the correlation of miR38R and miR3bR expression in individual hippocampal punches from high-dose animals for Construct B. The top two panels (FIG. 28A and FIG. 28B, respectively) each represent the correlated expression in individual male cynomolgus monkeys, and the bottom two panels (FIG. 28C and FIG. 28D, respectively) each represent the correlated expression in individual female cynomolgus monkeys. No significant difference was found between the sexes.

FIG. 29 is a graph showing the correlation of miR38R expression in hippocampal brain punches of low-, mid-, and high-dose cynomolgus monkeys versus the relative fold change of GRIK2 mRNA over the control group. Greater than 50% reduction in GRIK2 mRNA was observed with 200,000 miR38R copies/ng total RNA.

FIG. 30 is a graph showing the correlation of miR-3bR expression in hippocampal brain punches of low-, mid-, and high-dose cynomolgus monkeys versus relative fold change of GRIK2 mRNA over the control group. Greater than 50% reduction in GRIK2 mRNA was observed with 200,000 miR-3bR copies/ng total RNA.

FIG. 31 is a real time MRI image of vector administration in the hippocampus of a cynomolgus monkey.

FIGS. 32A and 32B are a set of graphs showing miRNA expression (FIG. 32A) and seizure count (FIG. 32B) following administration via bilateral injection of 2 μl/hippocampus of Construct B in pilocarpine mice.

FIGS. 33A and 33B show data collected post-administration of different doses of Construct B in a cynomolgus monkey. FIG. 33A is a map of a brain punch taken from a cynomolgus monkey following administration of different doses of Construct B via bilateral injection of 60 μl/hippocampus. The diluent was used as a control. FIG. 33B is an RNAscope image showing Construct B transduction at increasing magnification in a cynomolgus monkey hippocampus as observed in the slab 8 of the brain punch of FIG. 37A. Immunostaining for miR38 and GRIK2 showed localization (punctae) of Construct B to the hippocampal neurons.

FIG. 34 is a graph showing expression of miRNA (copies/10 pg total RNA) following administration of Construct B, G, or H in cynomolgus monkeys.

FIG. 35 is a graph showing the biodistribution (vg per μg tissue) of a high dose of Construct B in cynomolgus brain and CNS.

FIGS. 36A and 36B show miRNA biodistribution in cynomolgus monkeys following administration of Construct B. FIG. 36A is a graph showing the expression of miR38R (copies per ng host RNA) of a high dose of Construct B in cynomolgus brain and peripheral tissues. FIG. 36B is a graph showing the expression of miR3bR (copies per ng host RNA) of a high dose of Construct B in cynomolgus brain and CNS.

FIGS. 37A to 37F are a set of graphs showing quantification of vDNA, miR38R and miR3bR in pilocarpine mice administered Construct B or the diluent (37A, 37B, 37C) or in non-epileptic wild type mice administered Construct B or the diluent (37D, 37E, 37F). Results are presented as mean±SEM. Kruskal Wallis test followed by Dunn's multiple comparison, *P<0.05, **P<0.01, and ****P<0.0001.

FIGS. 38A to 38F show that vDNA levels correlate well with miR38R and miR3bR expression and that stable expression of miR38R and miR3bR correlates with stable knock down of GluK2 protein up to six months in vivo. FIGS. 38A and 38B show a correlation between vector copies and miR38R expression (FIG. 38A) or between vector copies and miR3bR expression (FIG. 38B) in pilocarpine treated mice up to six months after administration of Construct B. FIG. 38C is a graph showing GluK2 protein expression up to 6 months after administration of Construct B or diluent in pilocarpine mice.

FIGS. 38D and 38E show the correlation between vector copies and miR38R expression (FIG. 38D) or vector copies and miR3bR expression (FIG. 38E) in non-epileptic wild type mice up to six months after administration of Construct B. FIG. 38F is a graph showing GluK2 protein expression up to 6 months after administration of Construct B or diluent in non-epileptic wild type mice. Results are presented as mean±SEM. **P<0.0001 one-way ANOVA, main effect of Construct B and *P<0.05, one-way ANOVA, followed by Dunnett's multiple comparison.

FIG. 39 shows Construct B vDNA biodistribution in Cynomolgus monkeys after 6 months. For each tissue, vDNA levels were assessed at three doses. Each bar (three per tissue) shows the vDNA level of one dose±SD, the top one being the highest, middle being the medium and bottom one being the lowest dose. In some cases, no vDNA was detected.

FIG. 40 shows the correlation between combined miRNA expression (miR38R and miR3bR) versus Construct B vDNA in hippocampal brain punches of Cynomolgus monkeys after 6 months administration.

FIGS. 41A and 41B show miRNA expression in brain punches from Cynomolgus monkeys. In FIG. 41A, miR38R expression is down in brain tissues of Cynomolgus monkeys 6 months after administration of Construct B. In FIG. 41B, miR3bR expression is down in brain tissues of Cynomolgus monkeys 6 months after administration of Construct B. For each tissue, vDNA levels were assessed at three doses. Each bar (three per tissue) shows the miRNA expression of one dose±SD, the top one being the highest, middle being the medium and bottom one being the lowest dose. In some cases, no miRNA was detected.

FIG. 42 shows combined miRNA in hippocampus brain punches of Cynomolgus monkeys at each dose level and showing distance form the injection site from left to right (right being the furthest from injection site).

FIG. 43 shows the relationship between GRIK2 mRNA and combined miRNA expression showing a good correlation that increased miRNA expression leads to decreased GRIK2 mRNA expression in Cynomolgus monkeys 6 months after administration of Construct B.

### DETAILED DESCRIPTION

Described herein are compositions and methods for the treatment of an epilepsy, such as, e.g., a temporal lobe epilepsy (TLE; e.g., TLE that is refractory to or non-responsive to treatment with, e.g., anti-epileptic drugs) or a focal epilepsy, in a human subject (e.g., a human subject diagnosed with or exhibiting one or more symptoms of epilepsy). The compositions include a vector, such as a viral vector, e.g., an adeno-associated viral (AAV) vector (e.g., an AAV9 vector) with a nucleic acid sequence having at least 95%, at least 96%, at least 97%, at least 98%, at least 99%, or more sequence identity to SEQ ID NO: 2, 7, 8, or 16 (e.g., SEQ ID NO: 2 or 16, such as SEQ ID NO: 2).

The nucleic acid sequence encodes inhibitory ribopolynucleotides (e.g., two different ribopolynucleotides, e.g., miR3bR (SEQ ID NO: 14) and miR38R (SEQ ID NO: 15), or a dual miRNA including miR3bR and miR38R (SEQ ID NO: 13 or 16)), each of which inhibit glutamate ionotropic receptor kainate type subunit 2 (GRIK2). The vector (e.g., an AAV vector) is formulated for administration at a dosage that provides, e.g., from about 1×10¹¹ vector genomes (vg)/mL to about 1.0×10¹³ vg/mL, and optionally in a volume of, e.g., 3.0 mL or less, such as, e.g., 1.8 mL or less (e.g., 0.1 mL to about 1.8 mL). Administration of the vector (e.g., an AAV vector), or a composition containing the vector (e.g., an AAV vector), ameliorates one or more symptoms of epilepsy, for example, without eliciting an adverse effect. Administration of the vector (e.g., an AAV vector), and subsequent expression of the inhibitory ribopolynucleotides therefrom in transduced cells: (1) reduces the level of Grik2 in the brain (e.g., the hippocampus) of the subject for at least 28 days (for example, for 1-5 years, 1-10 years, 1-20 years, or 1-30 years, or for the life of the subject), (2) reduces the level of GluK2 in the brain (e.g., the hippocampus) of the subject for at least 28 days (for example, for 1-5 years, 1-10 years, 1-20 years, or 1-30 years, or for the life of the subject), (3) produces a level of expression of the vector (e.g., the AAV vector) at 10% or less in the dorsal root ganglion (DRG), blood, cerebral spinal fluid (CSF), and peripheral tissues (e.g., liver and heart) relative to the expression in the transduced neurons (e.g., transduced hippocampal neurons) of the subject prior to administration of the AAV, and/or (4) maintains Grik2 and Gluk2 reduction in the brain (e.g., hippocampus) of the subject for at least 1 year (for example, for 1-5 years, 1-10 years, 1-20 years, or 1-30 years, or, e.g., for the lifetime of the subject). The expression of the inhibitory ribopolynucleotides in transduced cells also (a) reduces the number of seizures per day, per week, per month, or per year and/or reduces epileptiform discharges in the subject, for example, as measured by an electroencephalogram and standardized to seizure frequency per 30 days or more, (b) improves the subject's measurements on routine laboratory parameters, such as hematology, biochemistry, coagulation, and urinalysis parameters, within at least 4 weeks, at least 5 weeks, at least 6 weeks, at least 7 weeks, at least 8 weeks, at least 3 months, at least 4 months, at least 5 months, at least 6 months, or at least 1 year (e.g., at least 2 years, 3 years, 4 years, 5 years, 10 years, 15 years, or 20 years, or for the life of the subject) after administration of the vector (e.g., an AAV vector) or the administration of a composition containing the vector (e.g., an AAV vector), (c) reduces frequency of interictal discharges, as measured by an electroencephalogram (EEG), (d) reduces aberrant neurological behavior by the subject, and/or (e) produces no adverse effects after 4 weeks.

For example, the AAV vector may be administered to the subject intra-parenchymally in an amount of from about 1×10¹¹ vector genomes (vg)/mL to about 1.0×10¹³ vg/mL, in a volume of, e.g., 3.0 mL or less, such as, e.g., 1.8 mL or less. The vector (e.g., an AAV vector) composition may be administered in a single dose per hemisphere (e.g., in a single dose to one or each hemisphere of the brain of the subject) by advancing a needle through the brain (e.g., the hippocampus) and dispensing a volume of the composition at between 1-10 focal sites (e.g., 5 or fewer sites, such as 5 sites) within the brain (e.g., the hippocampus).

Administration can be monitored or verified using, e.g., magnetic resonance imaging (MRI) or positron emission tomography (PET) scan. The total volume of the single dose of the composition may be divided (e.g., equally) by the number of focal sites. For example, administration of a volume of 1.8 mL of the composition at five distinct focal sites could involve administration of 360 μL of the composition at each focal site. The method of administration may include MRI-guided convection enhanced delivery (CED) using a CLEARPOINT® Neuro System and SMARTFLOW® cannula.

### GRIK2

GRIK2 is a gene encoding an ionotropic glutamate receptor subunit, GluK2, that is activated by the endogenous agonist glutamate and can also be selectively activated by the agonist kainate. GluK2-containing kainate receptors (KARs), like other ionotropic glutamate receptors, exhibit fast ligand gating by glutamate, which acts by opening a cation channel pore permeable to sodium and potassium. KAR complexes can be assembled from several subunits as heteromeric or homomeric assemblies of KAR subunits. Such receptors feature an extracellular N-terminus and a large peptide loop that together form the ligand-binding domain and an intracellular C-terminus. The ionotropic glutamate receptor complex itself acts as a ligand-gated ion channel, and upon binding glutamate mediates the passage of charged ions across the neuronal membrane. Generally, KARs are multimeric assemblies of GluK1, 2 and/or 3 (previously named GluR5, GluR6 and GluR7, respectively), GluK4 (KA1) and GluK5 (KA2) subunits (Collingridge, Neuropharmacology. 2009 January; 56(1):2-5). The various combinations of subunits involved in a KAR complex are often determined by RNA splicing and/or RNA editing (e.g., conversion of adenosine to inosine by adenosine deaminases) of mRNA encoding a particular KAR subunit. Furthermore, such RNA modification may impact the properties of the receptor, such as, e.g., altering calcium permeability of the channel. Increased activity of kainate receptors is known to be epileptogenic. GluK2-containing KARs are suitable targets for modulation of ionotropic glutamate receptor activity and subsequently amelioration of symptoms related to epileptogenesis (Peret et al. Cell Reports. 8(2): 347-354.2014).

### Focal Epilepsy

Epileptogenesis is a process that leads to the establishment of epilepsy and which may appear latent while cellular, molecular, and morphological changes leading to pathological neuronal network reorganization occur. Focal epilepsies are characterized by seizures arising from a specific lobe of the brain. Focal seizures are most common in people who have had head injuries, birth abnormalities of their brain, febrile seizures in childhood, infections of their brain (encephalitis), strokes, brain tumors, or other conditions that affect their brain. Focal epilepsies include idiopathic location-related epilepsies, frontal lobe epilepsy, temporal lobe epilepsy, parietal love epilepsy, and occipital lobe epilepsy. Methods of the disclosure can be used to treat focal epilepsy.

### Temporal Lobe Epilepsy

Temporal lobe epilepsy (TLE) is the most common form of focal epilepsy, affecting 6 of every 10 people with focal epilepsy. Seizures in TLE start in one or both temporal lobes. The hippocampus, including the DG, has been identified as a brain region particularly susceptible to damage that leads to TLE, and, in some instances, has been associated with treatment-resistant (i.e., refractory) epilepsy (Jarero-Basulto et al., Pharmaceuticals 11(1):17, 2018). An amplification of excitatory glutamatergic signaling may facilitate spontaneous seizures (Kuruba et al., Epilepsy Behav. 14 (Suppl. 1): 65-73, 2009).

Clinical management of TLE is notoriously difficult, with at least one third of TLE patients being unable to have adequate control of debilitating seizures using available medications. These patients often experience recurrent epileptic seizures that are refractory to treatment. The compositions and methods described herein can be used to treat the underlying molecular pathophysiology that leads to the development and progression of TLE.

### Methods of Treatment

The compositions described herein (e.g., an inhibitory ribopolynucleotide or an AAV vector (e.g., an AAV9 vector) containing the same) can be used for the treatment of epilepsy (e.g., focal epilepsy or TLE) or symptoms of epilepsy (e.g., seizures) by targeting GRIK2 mRNA and reducing the expression of GluK2 in neurons, which promotes a reduction in spontaneous epileptiform discharges in neuronal circuits (e.g., hippocampal circuits). As such, the compositions and methods described herein target the physiological cause of the disease and can be used for therapy.

### Dosing

The inhibitory ribopolynucleotide molecule (e.g., SEQ ID NO: 2, 7, 8, or 16 (e.g., SEQ ID NO: 2 or 16, such as SEQ ID NO: 2), or a variant thereof with at least 95% or greater sequence identity thereto), or a vector, e.g., a viral vector, such as an AAV vector (e.g., an AAV9 vector) containing the same, of the disclosure may be administered in an amount and for a time effective to result in one or more of (e.g., 2 or more, 3 or more, 4 or more of): (a) a decrease in the level of GRIK2 mRNA and/or GluK2 protein in a cell (e.g., a DG) of the subject, (b) delayed onset of the disorder (e.g., epilepsy, such as focal epilepsy or TLE), or one or more symptoms thereof, (c) increased survival of subject, (d) increased progression free survival of a subject, (e) a change in GluK2 protein function or KAR function, (f) reduce risk of seizure recurrence; (g) reduction of excitotoxicity and associated neuronal cell death in the CNS; (h) restoration of a physiological excitation-inhibition balance in the affected region of the CNS (e.g., the hippocampus); and/or (i) reduction in one or more symptoms of a epilepsy (e.g., frequency, duration, or intensity of epileptic seizures). The AAV vector (e.g., an AAV9 vector) including a nucleic acid molecule (e.g., SEQ ID NO: 2, 7, 8, or 16 (e.g., SEQ ID NO: 2 or 16, such as SEQ ID NO: 2), or a variant thereof with at least 95% or greater sequence identity thereto encoding an inhibitory ribopolynucleotide molecule (e.g., dual ribopolynucleotide molecules, miR3bR and miR38R, e.g., SEQ ID NO: 13 or 16) can be administered to the subject in an amount of about 1×10⁹ vg, 2×10⁹ vg, 3×10⁹ vg, 4×10⁹ vg, 5×10⁹ vg, 6×10⁹ vg, 7×10⁹ vg, 8×10⁹ vg, 9×10⁹ vg, 1×10¹⁰ vg, 2×10¹⁰ vg, 3×10¹⁰ vg, 4×10¹⁰ vg, 5×10¹⁰ vg, 6×10¹⁰ vg, 7×10¹⁰ vg, 8×10¹⁰ vg, 9×10¹⁰ vg, 1×10¹¹ vg, 2×10¹¹ vg, 3×10¹¹ vg, 4×10¹¹ vg, 5×10¹¹ vg, 6×10¹¹ vg, 7×10¹¹ vg, 8×10¹¹ vg, 9×10¹¹ vg, 1×10¹² vg, 2×10¹² vg, 3×10¹² vg, 4×10¹² vg, 5×10¹² vg, 6×10¹² vg, 7×10¹² vg, 8×10¹² vg, 9×10¹² vg, or 1×10¹³ vg.

The disclosed compositions can be administered in amounts determined to be appropriate by those of skill in the art. For example, the AAV vector (e.g., an AAV9 vector) containing a nucleic acid molecule (e.g., SEQ ID NO: 2, 7, 8, or 16 (e.g., SEQ ID NO: 2 or 16, such as SEQ ID NO: 2), or a variant thereof with at least 95% or greater sequence identity thereto) encoding an inhibitory ribopolynucleotide molecule (e.g., dual ribopolynucleotide molecules, miR3bR and miR38R, e.g., SEQ ID NO: 13 or 16) can be administered at a dose of from about 1×10¹¹ vector genomes (vg)/mL to about 1.0×10¹³ vg/mL (e.g., from about 2×10¹¹ vg/mL to about 9×10¹² vg/mL, from about 3×10¹¹ vg/mL to about 8×10¹² vg/mL, from about 4×10¹¹ vg/mL to about 7×10¹² vg/mL, from about 5×10¹¹ vg/mL to about 6×10¹² vg/mL, from about 6×10¹¹ vg/mL to about 5×10¹² vg/mL, from about 7×10¹¹ vg/mL to about 4×10¹² vg/mL, from about 8×10¹¹ vg/mL to about 3×10¹¹ vg/mL, from about 9×10¹¹ vg/mL to about 2×10¹² vg/mL, or about 1×10¹² vg/mL). In some embodiments, the AAV vector is administered at a dose of from about 2×10¹¹ vg/mL to about 9×10¹² vg/mL. In some embodiments, the AAV vector is administered at a dose of from about 3×10¹¹ vg/mL to about 8×10¹² vg/mL. In some embodiments, the AAV vector is administered at a dose of from about 4×10¹¹ vg/mL to about 7×10¹² vg/mL. In some embodiments, the AAV vector is administered at a dose of from about 5×10¹¹ vg/mL to about 6×10¹² vg/mL. In some embodiments, the AAV vector is administered at a dose of from about 6×10¹¹ vg/mL to about 5×10¹² vg/mL. In some embodiments, the AAV vector is administered at a dose from about 7×10¹¹ vg/mL to about 4×10¹² vg/mL. In some embodiments, the AAV vector is administered at a dose of from about 8×10¹¹ vg/mL to about 3×10¹¹ vg/mL. In some embodiments, the AAV vector is administered at a dose of from about 9×10¹¹ vg/mL to about 2×10¹² vg/mL. In some embodiments, the AAV vector is administered at a dose of about 1×10¹² vg/mL. In some embodiments, the AAV vector is administered at a dose of about 1×10¹¹ vg/mL. In some embodiments, the AAV vector is administered at a dose of about 2×10¹¹ vg/mL. In some embodiments, the AAV vector is administered at a dose of about 3×10¹¹ vg/mL. In some embodiments, the AAV vector is administered at a dose of about 4×10¹¹ vg/mL. In some embodiments, the AAV vector is administered at a dose of about 5×10¹¹ vg/mL. In some embodiments, the AAV vector is administered at a dose of about 6×10¹¹ vg/mL. In some embodiments, the AAV vector is administered at a dose of about 7×10¹¹ vg/mL. In some embodiments, the AAV vector is administered at a dose of about 8×10¹¹ vg/mL. In some embodiments, the AAV vector is administered at a dose of about 9×10¹¹ vg/mL. In some embodiments, the AAV vector is administered at a dose of about 1×10¹² vg/mL. In some embodiments, the AAV vector is administered at a dose of about 2×10¹² vg/mL. In some embodiments, the AAV vector is administered at a dose of about 3×10¹² vg/mL. In some embodiments, the AAV vector is administered at a dose of about 4×10¹² vg/mL. In some embodiments, the AAV vector is administered at a dose of about 5×10¹² vg/mL. In some embodiments, the AAV vector is administered at a dose of about 6×10¹² vg/mL. In some embodiments, the AAV vector is administered at a dose of about 7×10¹² vg/mL. In some embodiments, the AAV vector is administered at a dose of about 8×10¹² vg/mL. In some embodiments, the AAV vector is administered at a dose of about 9×10¹² vg/mL. In some embodiments, the AAV vector is administered at a dose of about 1×10¹³ vg/mL.

In some embodiments, the AAV vector is administered at a dose of 3×10⁸ vg/mm³ hippocampus to about 1.2×10⁹ vg/mm³ hippocampus (e.g., 4×10⁸ vg/mm³ hippocampus to 1×10⁹ vg/mm³ hippocampus, 5×10⁸ vg/mm³ hippocampus to 9×10⁸ vg/mm³ hippocampus, 6×10⁸ vg/mm³ hippocampus to 8×10⁸ vg/mm³ hippocampus, or 7×10⁸ vg/mm³ hippocampus). In some embodiments, the AAV vector is administered at a dose of 4×10⁸ vg/mm³ hippocampus to 1×10⁹ vg/mm³ hippocampus. In some embodiments, the AAV vector is administered at a dose of 5×10⁸ vg/mm³ hippocampus to 9×10⁸ vg/mm³ hippocampus. In some embodiments, the AAV vector is administered at a dose of 6×10⁸ vg/mm³ hippocampus to 8×10⁸ vg/mm³ hippocampus. In some embodiments, the AAV vector is administered at a dose of 7×10⁸ vg/mm³ hippocampus. In some embodiments, the AAV vector is administered at a dose of 3×10⁸ vg/mm³ hippocampus. In some embodiments, the AAV vector is administered at a dose of 4×10⁸ vg/mm³ hippocampus. In some embodiments, the AAV vector is administered at a dose of 5×10⁸ vg/mm³ hippocampus. In some embodiments, the AAV vector is administered at a dose of 6×10⁸ vg/mm³ hippocampus. In some embodiments, the AAV vector is administered at a dose of 7×10⁸ vg/mm³ hippocampus. In some embodiments, the AAV vector is administered at a dose of 8×10⁸ vg/mm³ hippocampus. In some embodiments, the AAV vector is administered at a dose of 9×10⁸ vg/mm³ hippocampus. In some embodiments, the AAV vector is administered at a dose of 1×10⁹ vg/mm³ hippocampus.

In some embodiments, the AAV vector is administered at a dose of 9×10¹¹ total vg/hippocampus to about 3.6×10¹² total vg/hippocampus (e.g., 1×10¹² total vg/hippocampus to 3×10¹² total vg/hippocampus, or 2×10¹² total vg/hippocampus). In some embodiments, the AAV vector is administered at a dose of 1×10¹² total vg/hippocampus to 3×10¹² total vg/hippocampus. In some embodiments, the AAV vector is administered at a dose of 2×10¹² total vg/hippocampus. In some embodiments, the AAV vector is administered at a dose of 9×10¹¹ total vg/hippocampus. In some embodiments, the AAV vector is administered at a dose of 1×10¹² total vg/hippocampus. In some embodiments, the AAV vector is administered at a dose of 2×10¹² total vg/hippocampus. In some embodiments, the AAV vector is administered at a dose of 3×10¹² total vg/hippocampus. In some embodiments, the AAV vector is administered at a dose of 3.6×10¹² total vg/hippocampus. These doses can also be administered in the indicated amounts to a region of the brain other than the hippocampus (either unilaterally or bilaterally) to treat epilepsy (e.g., a focal epilepsy) that occurs, e.g., outside of the hippocampus.

In some embodiments, the single dose is administered to the subject in a total volume of 0.1 mL to 3 mL (e.g., 0.1 mL to 3 mL, 0.5 mL to 2 mL, 1 mL to 1.75 mL, or 1.25 mL to 1.5 mL). In some embodiments, the single dose is administered to the subject in a total volume of 0.5 mL to 1.8 mL (e.g., 0.5 mL to 1.8 mL, 0.75 mL to 1.75 mL, 1.0 mL to 1.5 mL, or 1.25 mL). In some embodiments, the single dose is administered to the subject in a total volume of 0.75 mL to 1.75 mL. In some embodiments, the single dose is administered to the subject in a total volume of 1.0 mL to 1.5 mL. In some embodiments, the single dose is administered to the subject in a total volume of 1.25 mL. In some embodiments, the single dose is administered to the subject in a total volume of 0.5 mL. In some embodiments, the single dose is administered to the subject in a total volume of 0.75 mL. In some embodiments, the single dose is administered to the subject in a total volume of 1.0 mL. In some embodiments, the single dose is administered to the subject in a total volume of 1.25 mL. In some embodiments, the single dose is administered to the subject in a total volume of 1.5 mL. In some embodiments, the single dose is administered to the subject in a total volume of 1.75 mL. In some embodiments, the single dose is administered to the subject in a total volume of about 1.8 mL (e.g., 1.8 mL).

In some embodiments, the total volume of the single dose is about 20% to about 70% (e.g., 50%) of the subject's hippocampal volume (or, e.g., a volume of a region of the brain other than the hippocampus). Subjects diagnosed with TLE suffer from hippocampal atrophy and exhibit a reduction in hippocampal volume relative to age matched healthy controls of about 25%. Hippocampal volume may be determined by nomogram assessment, for example, as described in Nobis et al. (NeuroImage: Clinical. 23, 2019). In some embodiments, the total volume of the single dose is no lower than 0.5 mL. In some embodiments, the total volume of the single dose does not exceed 1.8 mL.

Optionally, the disclosed agents may be administered as part of a pharmaceutically acceptable composition suitable for delivery to a subject, as is described herein. The disclosed agents are included within these compositions in amounts sufficient to provide a desired dosage and/or elicit a therapeutically beneficial effect, as can be readily determined by those of skill in the art.

The disclosed compositions described herein may be administered in an amount (e.g., an effective amount) and for a time sufficient to treat the subject or to effect one of the outcomes described above (e.g., a reduction in one or more symptoms of disease in the subject). The disclosed compositions may be administered once or more than once. The disclosed compositions may be administered once yearly. Subjects may be evaluated for treatment efficacy 1 month, 2 months, 3 months, 4 months, 5 months, 6 months or more following administration of a composition of the disclosure depending on the composition and the route of administration used for treatment. Methods of evaluating treatment efficacy are disclosed herein (see, e.g., the section titled “Pharmaceutical Uses”). Depending on the outcome of the evaluation, treatment may be continued or ceased (and, e.g., if ceased, treatment may be resumed at a later time, if desired), treatment frequency or dosage may change, or the patient may be treated with a different disclosed composition. Subjects may be monitored after treatment for a period of time (e.g., over the course of 1, 2, 3, 4, 5, 6, 7, 8, 9, 10, 11, 12 months, or more, once quarterly, bi-annually, yearly, or once every 2, 3, 4, or 5 years or more). If the effectiveness of the treatment appears to wane or diminish, treatment with the disclosed composition may be repeated one or more times, as needed (e.g., until symptoms of the disease or condition (e.g., seizures) are alleviated). Treatment may be administered multiple times over the life of the subject, e.g., depending on the severity and nature of the disease or condition being treated in the subject. For example, a subject diagnosed with TLE and treated with a composition disclosed herein may be given one or more (e.g., 1, 2, 3, 4, 5, 6, 7, 8, 9, 10, or more) additional treatments if initial or subsequent rounds of treatment do not elicit a therapeutic benefit (e.g., reduction of any one of the symptoms disclosed herein or a reduction in the levels of GRIK2 mRNA or GluK2 protein levels in the afflicted brain region of the subject).

Inhibitory Polynucleotides Targeting GRIK2 mRNA

The compositions described herein, which are AAV vectors including polynucleotides encoding inhibitory ribonucleic acid constructs (e.g., inhibitory ribopolynucleotides or nucleic acid vectors encoding the same) that target GRIK2, can be administered according to the methods described herein to treat an epilepsy, such as focal epilepsy or TLE.

The inhibitory ribopolynucleotide (e.g., a ribopolynucleotide including a nucleic acid sequence with at least 95% sequence identity to SEQ ID NO: 2, 7, 8, or 16 (e.g., SEQ ID NO: 2 or 16, such as SEQ ID NO: 2)) may inhibit the expression of GluK2 by causing the degradation of GRIK2 mRNA in a cell (e.g., a neuron, such as, e.g., a hippocampal neuron, such as, e.g., a hippocampal neuron of the dentate gyrus, such as, e.g., a dentate granule cell (DGC), or a glutamatergic pyramidal neuron), thereby preventing translation of the Grik2 mRNA into a functional GluK2 protein. In some embodiments, the ribopolynucleotide includes a nucleic acid sequence with at least 95% (e.g., at least 95%, 96%, 97%, 98%, 99%, or more (e.g., 100%)) sequence identity to SEQ ID NO: 2. In some embodiments, the ribopolynucleotide includes the nucleic acid sequence of SEQ ID NO: 2. In some embodiments, the ribopolynucleotide includes a nucleic acid sequence with at least 95% (e.g., at least 95%, 96%, 97%, 98%, 99%, or more (e.g., 100%)) sequence identity to SEQ ID NO: 16. In some embodiments, the ribopolynucleotide includes the nucleic acid sequence of SEQ ID NO: 16. In some embodiments, the ribopolynucleotide includes a nucleic acid sequence with at least 95% (e.g., at least 95%, 96%, 97%, 98%, 99%, or more (e.g., 100%)) sequence identity to SEQ ID NO: 7. In some embodiments, the ribopolynucleotide includes the nucleic acid sequence of SEQ ID NO: 7. In some embodiments, the ribopolynucleotide includes a nucleic acid sequence with at least 95% (e.g., at least 95%, 96%, 97%, 98%, 99%, or more (e.g., 100%)) sequence identity to SEQ ID NO: 8. In some embodiments, the ribopolynucleotide includes the nucleic acid sequence of SEQ ID NO: 8.

The inhibitory ribopolynucleotide targeting the GRIK2 mRNA disclosed herein may act to decrease the frequency of or completely inhibit the occurrence of epileptic brain activity (e.g., epileptiform discharges) in one or more brain regions. Such brain regions may include, but are not limited to the mesial temporal lobe, lateral temporal lobe, frontal lobe, or more specifically, hippocampus (e.g., DG, CA1, CA2, CA3, subiculum) or neocortex. Due to the aberrant expression of GluK2-containing KARs in rMF-DGCs of the DG, the occurrence of epileptic brain activity, as well as the occurrence of interictal discharges, may be inhibited in the DG.

Accordingly, the disclosure provides methods and compositions for reducing epileptiform discharges in a CNS cell (e.g., a DGC) by contacting the cell with an effective amount of an AAV vector (e.g., an AAV9) encoding an inhibitory ribopolynucleotide with at least 95% (e.g., at least 95%, 96%, 97%, 98%, 99%, or more (e.g., 100%)) sequence identity to SEQ ID NO: 2, 7, 8, or 16. In some embodiments, the inhibitory ribopolynucleotide has at least 96% sequence identity to SEQ ID NO: 2, 7, 8, or 16. In some embodiments, the inhibitory ribopolynucleotide has at least 97% sequence identity to SEQ ID NO: 2, 7, 8, or 16. In some embodiments, the inhibitory ribopolynucleotide has at least 98% sequence identity to SEQ ID NO: 2, 7, 8, or 16. In some embodiments, the inhibitory ribopolynucleotide has at least 99% sequence identity to SEQ ID NO: 2, 7, 8, or 16. In some embodiments, the inhibitory ribopolynucleotide has the sequence of SEQ ID NO: 2, 7, 8, or 16.

In some embodiments, the disclosure provides methods and compositions for reducing epileptiform discharges in a CNS cell (e.g., a DGC) by contacting the cell with an effective amount of an AAV vector (e.g., an AAV9) encoding an inhibitory ribopolynucleotide with at least 95% (e.g., at least 95%, 96%, 97%, 98%, 99%, or more (e.g., 100%)) sequence identity to SEQ ID NO: 2. In some embodiments, the inhibitory ribopolynucleotide has at least 96% sequence identity to SEQ ID NO: 2. In some embodiments, the inhibitory ribopolynucleotide has at least 97% sequence identity to SEQ ID NO: 2. In some embodiments, the inhibitory ribopolynucleotide has at least 98% sequence identity to SEQ ID NO: 2. In some embodiments, the inhibitory ribopolynucleotide has at least 99% sequence identity to SEQ ID NO: 2. In some embodiments, the inhibitory ribopolynucleotide has the sequence of SEQ ID NO: 2.

In some embodiments, the disclosure provides methods and compositions for reducing epileptiform discharges in a CNS cell (e.g., a DGC) by contacting the cell with an effective amount of an AAV vector (e.g., an AAV9) encoding an inhibitory ribopolynucleotide with at least 95% (e.g., at least 95%, 96%, 97%, 98%, 99%, or more (e.g., 100%)) sequence identity to SEQ ID NO: 16. In some embodiments, the inhibitory ribopolynucleotide has at least 96% sequence identity to SEQ ID NO: 16. In some embodiments, the inhibitory ribopolynucleotide has at least 97% sequence identity to SEQ ID NO: 16. In some embodiments, the inhibitory ribopolynucleotide has at least 98% sequence identity to SEQ ID NO: 16. In some embodiments, the inhibitory ribopolynucleotide has at least 99% sequence identity to SEQ ID NO: 16. In some embodiments, the inhibitory ribopolynucleotide has the sequence of SEQ ID NO: 16. In some embodiments, the ribopolynucleotide includes or encodes a miRNA.

In some embodiments, the disclosure provides methods and compositions for reducing epileptiform discharges in a CNS cell (e.g., a DGC) by contacting the cell with an effective amount of an AAV vector (e.g., an AAV9) encoding an inhibitory ribopolynucleotide with at least 95% (e.g., at least 95%, 96%, 97%, 98%, 99%, or more (e.g., 100%)) sequence identity to SEQ ID NO: 7. In some embodiments, the inhibitory ribopolynucleotide has at least 96% sequence identity to SEQ ID NO: 7. In some embodiments, the inhibitory ribopolynucleotide has at least 97% sequence identity to SEQ ID NO: 7. In some embodiments, the inhibitory ribopolynucleotide has at least 98% sequence identity to SEQ ID NO: 7. In some embodiments, the inhibitory ribopolynucleotide has at least 99% sequence identity to SEQ ID NO: 7. In some embodiments, the inhibitory ribopolynucleotide has the sequence of SEQ ID NO: 7. In some embodiments, the ribopolynucleotide includes or encodes a miRNA.

In some embodiments, the disclosure provides methods and compositions for reducing epileptiform discharges in a CNS cell (e.g., a DGC) by contacting the cell with an effective amount of an AAV vector (e.g., an AAV9) encoding an inhibitory ribopolynucleotide with at least 95% (e.g., at least 95%, 96%, 97%, 98%, 99%, or more (e.g., 100%)) sequence identity to SEQ ID NO: 8. In some embodiments, the inhibitory ribopolynucleotide has at least 96% sequence identity to SEQ ID NO: 8. In some embodiments, the inhibitory ribopolynucleotide has at least 97% sequence identity to SEQ ID NO: 8. In some embodiments, the inhibitory ribopolynucleotide has at least 98% sequence identity to SEQ ID NO: 8. In some embodiments, the inhibitory ribopolynucleotide has at least 99% sequence identity to SEQ ID NO: 8. In some embodiments, the inhibitory ribopolynucleotide has the sequence of SEQ ID NO: 8. In some embodiments, the ribopolynucleotide includes or encodes a miRNA.

In some embodiments, the ribopolynucleotide includes dual miRNAs, such as miR3bR and miR38R (e.g., SEQ ID NO: 13), which includes the sequences of miRNAs such as miR3bR (e.g., SEQ ID NO: 14) and miR38R (e.g., SEQ ID NO: 15). In some embodiments, the ribopolynucleotide has the sequence of SEQ ID NO: 13. In some embodiments, the ribopolynucleotide has at least 95% (e.g., at least 96%, at least 97%, at least 98%, at least 99%, or more (e.g., 100%)) sequence identity to the nucleotide sequence of SEQ ID NO: 13. In some embodiments, the ribopolynucleotide has at least 96% sequence identity to the nucleotide sequence of SEQ ID NO: 13. In some embodiments, the ribopolynucleotide has at least 97% sequence identity to the nucleotide sequence of SEQ ID NO: 13. In some embodiments, the ribopolynucleotide has at least 98% sequence identity to the nucleotide sequence of SEQ ID NO: 13. In some embodiments, the ribopolynucleotide has at least 99% sequence identity to the nucleotide sequence of SEQ ID NO: 13.

In some embodiments, the ribopolynucleotide includes or encodes a miRNA. In some embodiments, the ribopolynucleotide includes dual miRNAs, such as miR3bR and miR38R (e.g., as set forth in SEQ ID NO: 2 or 16), which includes the sequences of miRNAs such as miR3bR (e.g., SEQ ID NO: 14) and miR38R (e.g., SEQ ID NO: 15).

In some embodiments, the miRNA includes the sequence of miR3bR (e.g., SEQ ID NO: 14). In some embodiments, the miRNA includes a sequence having at least 95% (e.g., at least 96%, at least 97%, at least 98%, at least 99%, or more (e.g., 100%)) sequence identity to the nucleotide sequence of SEQ ID NO: 14. In some embodiments, the miRNA includes a sequence having at least 96% sequence identity to the nucleotide sequence of SEQ ID NO: 14. In some embodiments, the miRNA includes a sequence having at least 97% sequence identity to the nucleotide sequence of SEQ ID NO: 14. In some embodiments, the miRNA includes a sequence having at least 98% sequence identity to the nucleotide sequence of SEQ ID NO: 14. In some embodiments, the miRNA includes a sequence having at least 99% sequence identity to the nucleotide sequence of SEQ ID NO: 14.

In some embodiments, the miRNA includes the sequence of miR38R (e.g., SEQ ID NO: 15). In some embodiments, the miRNA includes a sequence having at least 95% (e.g., at least 96%, at least 97%, at least 98%, at least 99%, or more (e.g., 100%)) sequence identity to the nucleotide sequence of SEQ ID NO: 15. In some embodiments, the miRNA includes a sequence having at least 96% sequence identity to the nucleotide sequence of SEQ ID NO: 15. In some embodiments, the miRNA includes a sequence having at least 97% sequence identity to the nucleotide sequence of SEQ ID NO: 15. In some embodiments, the miRNA includes a sequence having at least 98% sequence identity to the nucleotide sequence of SEQ ID NO: 15. In some embodiments, the miRNA includes a sequence having at least 99% sequence identity to the nucleotide sequence of SEQ ID NO: 15.

### Adeno-Associated Viral (AAV) Vectors Encoding GRIK2 Inhibitory Polynucleotides

Nucleic acids of the compositions (e.g., the nucleic acid of SEQ ID NO: 2, 7, 8, or 16 (e.g., SEQ ID NO: 2 or 16, such as SEQ ID NO: 2), and variants thereof with at least 95% sequence identity thereto and greater, e.g., with 96%, 97%, 98%, 99% or more sequence identity thereto) may be incorporated into a viral vector, such as a recombinant adeno-associated virus (AAV) vector, a lentiviral vector, a retroviral vector, or a herpes simplex vector, in order to facilitate their introduction into a cell (e.g., a neuron). AAVs useful in the compositions and methods described herein contain recombinant ribopolynucleotides that include (1) a heterologous sequence to be expressed and (2) viral sequences that facilitate integration and expression of the heterologous genes.

The ribopolynucleotides described herein can be incorporated into a virion (e.g., an AAV virion) in order to facilitate introduction of the nucleic acid or vector into a cell. Examples of AAVs that can be used to produce an AAV vector of the disclosure include, e.g., AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV-TT, AAV-DJ8, and AAV.HSC16. For targeting cells located in or delivered to the central nervous system, AAV2, AAV9, and AAV10 may be particularly useful. For example, any of these AAVs can be modified to include the nucleic acid of SEQ ID NO: 2, 7, 8, or 16 (e.g., SEQ ID NO: 2 or 16, such as SEQ ID NO: 2), and variants thereof with at least 95% sequence identity thereto and greater (e.g., with 96%, 97%, 98%, 99% or more sequence identity thereto). Construction and use of AAV vectors and AAV proteins of different serotypes are known in the art.

The disclosure relates an AAV vector for delivery of a heterologous ribopolynucleotide (e.g., SEQ ID NO: 2, 7, 8, or 16 (e.g., SEQ ID NO: 2 or 16, such as SEQ ID NO: 2)) that encodes an inhibitory RNA agent (e.g., miRNA, or shmiRNA) construct that specifically binds GRIK2 mRNA and inhibits expression of GluK2 protein in a cell. Accordingly, an object of the disclosure provides an AAV vector including an inhibitory ribopolynucleotide sequence that includes a nucleic acid sequence with at least 95% (e.g., at least 95%, 96%, 97%, 98%, 99%, or more (e.g., 100%)) sequence identity to the sequence of SEQ ID NO: 2, 7, 8, or 16 (e.g., SEQ ID NO: 2 or 16, such as SEQ ID NO: 2). In some embodiments, the ribopolynucleotide sequence includes a nucleic acid sequence with at least 96% sequence identity to the sequence of SEQ ID NO: 2, 7, 8, or 16 (e.g., SEQ ID NO: 2 or 16, such as SEQ ID NO: 2). In some embodiments, the ribopolynucleotide sequence includes a nucleic acid sequence with at least 97% sequence identity to the sequence of SEQ ID NO: 2, 7, 8, or 16 (e.g., SEQ ID NO: 2 or 16, such as SEQ ID NO: 2). In some embodiments, the ribopolynucleotide sequence includes a nucleic acid sequence with at least 98% sequence identity to the sequence of SEQ ID NO: 2, 7, 8, or 16 (e.g., SEQ ID NO: 2 or 16, such as SEQ ID NO: 2). In some embodiments, the ribopolynucleotide sequence includes a nucleic acid sequence with at least 99% sequence identity to the sequence of SEQ ID NO: 2, 7, 8, or 16 (e.g., SEQ ID NO: 2 or 16, such as SEQ ID NO: 2). In some embodiments, the ribopolynucleotide sequence includes the nucleic acid sequence of SEQ ID NO: 2, 7, 8, or 16 (e.g., SEQ ID NO: 2 or 16, such as SEQ ID NO: 2).

In some embodiments, the inhibitory RNA agent is a miRNA. In some embodiments, the AAV vector includes a dual miRNA ribopolynucleotide (e.g., SEQ ID NO: 13). For example, the AAV vector includes the miRNA miR3bR (e.g., SEQ ID NO: 14) and the miRNA miR38R (e.g., SEQ ID NO: 15). In some embodiments, the ribopolynucleotide has the sequence of SEQ ID NO: 13 or 16. In some embodiments, the ribopolynucleotide has at least 95% (e.g., at least 96%, at least 97%, at least 98%, at least 99%, or more (e.g., 100%)) sequence identity to the nucleotide sequence of SEQ ID NO: 13 or 16. In some embodiments, the ribopolynucleotide has at least 96% sequence identity to the nucleotide sequence of SEQ ID NO: 13 or 16. In some embodiments, the ribopolynucleotide has at least 97% sequence identity to the nucleotide sequence of SEQ ID NO: 13 or 16. In some embodiments, the ribopolynucleotide has at least 98% sequence identity to the nucleotide sequence of SEQ ID NO: 13. In some embodiments, the ribopolynucleotide has at least 99% sequence identity to the nucleotide sequence of SEQ ID NO: 13 or 16.

In some embodiments, the miRNA includes the sequence of miR3bR (e.g., SEQ ID NO: 14). In some embodiments, the miRNA includes a sequence having at least 95% (e.g., at least 96%, at least 97%, at least 98%, at least 99%, or more (e.g., 100%)) sequence identity to the nucleotide sequence of SEQ ID NO: 14. In some embodiments, the miRNA includes a sequence having at least 96% sequence identity to the nucleotide sequence of SEQ ID NO: 14. In some embodiments, the miRNA includes a sequence having at least 97% sequence identity to the nucleotide sequence of SEQ ID NO: 14. In some embodiments, the miRNA includes a sequence having at least 98% sequence identity to the nucleotide sequence of SEQ ID NO: 14. In some embodiments, the miRNA includes a sequence having at least 99% sequence identity to the nucleotide sequence of SEQ ID NO: 14.

In some embodiments, the miRNA includes the sequence of miR38R (e.g., SEQ ID NO: 15). In some embodiments, the miRNA includes a sequence having at least 95% (e.g., at least 96%, at least 97%, at least 98%, at least 99%, or more (e.g., 100%)) sequence identity to the nucleotide sequence of SEQ ID NO: 15. In some embodiments, the miRNA includes a sequence having at least 96% sequence identity to the nucleotide sequence of SEQ ID NO: 15. In some embodiments, the miRNA includes a sequence having at least 97% sequence identity to the nucleotide sequence of SEQ ID NO: 15. In some embodiments, the miRNA includes a sequence having at least 98% sequence identity to the nucleotide sequence of SEQ ID NO: 15. In some embodiments, the miRNA includes a sequence having at least 99% sequence identity to the nucleotide sequence of SEQ ID NO: 15.

### Pharmaceutical Uses

Disclosed herein are methods for the treatment of an epilepsy (e.g., focal epilepsy or TLE) in a subject diagnosed with or displaying one or more symptoms of epilepsy by administration of the compositions described herein (e.g., an inhibitory ribopolynucleotide (e.g., a dual miRNA construct, such as that of SEQ ID NO: 2 or 16, or a single miRNA construct, such as that of SEQ ID NO: 7 or 8, and variants thereof, as described herein) or an AAV vector encoding the same).

### Therapeutic Effects

Administration of the compositions (e.g., an inhibitory ribonucleotide or AAV vector encoding the same) described herein by the methods of the disclosure promotes expression of the miRNAs (e.g., miR3bR (SEQ ID NO: 14) and miR38R (SEQ ID NO: 15)) encoded by the inhibitory ribopolynucleotide at a level of 1×10³ to 1×10⁷ (e.g., 1×10⁴ to 1×10⁶, or 1×10⁵) copies/nanogram of RNA for both miR3bR and miR38R in a cell of the subject. In some embodiments, administration of the compositions described herein by the methods of the disclosure promotes expression of the miRNAs (e.g., miR3bR and miR38R) encoded by the inhibitory ribopolynucleotide at a level of 1×10⁴ to 1×10⁸ copies/nanogram of RNA for both miR3bR and miR38R in a cell of the subject (e.g., 1×10⁴ to 1×10⁶ copies/nanogram of RNA for both miR3bR and miR38R). In some embodiments, administration of the compositions described herein by the methods of the disclosure promotes expression of the miRNAs (e.g., miR3bR and miR38R) encoded by the inhibitory ribopolynucleotide at a level of 1×10⁵ copies/nanogram of RNA for both miR3bR and miR38R in a cell of the subject.

Administration of the compositions described herein by the methods of the disclosure promotes transduction of the AAV vector in neuronal cells of the subject, but not in the subject's DRG, blood, CSF, or peripheral tissues (e.g., liver and heart). For example, transduction of the AAV vector, and expression of the inhibitory ribopolynucleotide, in the subject's DRG, blood, CSF, or peripheral tissues (e.g., liver and heart) occurs at a level of 10% or less (e.g., 9%, 8%, 7%, 6%, 5%, 4%, 3%, 2%, 1%, or less) relative to transduction of the AAV vector, and expression of the inhibitory ribopolynucleotide in, a transduced cell of the brain (e.g., the hippocampus (e.g., in a hippocampal neuron)). In some embodiments, administration of the compositions described herein by the methods of the disclosure produces no transduction of the AAV vector in the subject's DRG, blood, CSF, or peripheral tissues (e.g., liver and heart).

In some embodiments, transduction of the AAV vector, and expression of the inhibitory ribopolynucleotide, in the subject's DRG, blood, CSF, or peripheral tissues (e.g., liver and heart) occurs at a level of 1×10⁶ ds vg/μg of DNA or ds vg/mL biofluid or less (e.g., 1×10⁵ ds vg/μg of DNA or ds vg/mL biofluid, 1×10⁴ ds vg/μg of DNA or ds vg/mL biofluid, 1×10³ ds vg/μg of DNA or ds vg/mL biofluid, 1×10² ds vg/μg of DNA or ds vg/mL biofluid, 10 ds vg/μg of DNA or ds vg/mL biofluid, or less). In some embodiments, transduction of the AAV vector, and expression of the inhibitory ribopolynucleotide, in the subject's DRG, blood, and/or CSF occurs at a level of 1×10⁵ ds vg/μg of DNA or ds vg/mL biofluid or less. In some embodiments, transduction of the AAV vector, and expression of the inhibitory ribopolynucleotide, in the subject's DRG, blood, and/or CSF occurs at a level of 1×10⁴ ds vg/μg of DNA or ds vg/mL biofluid or less. In some embodiments, transduction of the AAV vector, and expression of the inhibitory ribopolynucleotide, in the subject's DRG, blood, and/or CSF occurs at a level of 1×10³ ds vg/μg of DNA or ds vg/mL biofluid or less. In some embodiments, transduction of the AAV vector, and expression of the inhibitory ribopolynucleotide, in the subject's DRG, blood, and/or CSF occurs at a level of 1×10² ds vg/μg of DNA or ds vg/mL biofluid or less. In some embodiments, transduction of the AAV vector in, and expression of the inhibitory ribopolynucleotide, in the subject's DRG, blood, and/or CSF occurs at a level of 10 ds vg/μg of DNA or ds vg/mL biofluid or less.

Administration of the compositions described herein by the methods of the disclosure provides a reduction in the level of GRIK2 expression in a transduced cell in the brain (e.g., the hippocampus) of the subject. Accordingly, the level of GRIK2 expression in a transduced cell in the brain (e.g., the hippocampus) of the subject is reduced by at least 10%, at least at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or more, e.g., relative to a control AAV vector or relative to a cell in the brain (e.g., the hippocampus) of the subject that is not transduced. In some embodiments, the level of GRIK2 expression in a transduced cell in the brain (e.g., the hippocampus) of the subject is reduced by 5% to 60% (e.g., 10% to 50%, 20% to 40%, or 30%). In some embodiments, the level of GRIK2 expression in a transduced cell in the brain (e.g., the hippocampus) of the subject is reduced by 10% to 50%. In some embodiments, the level of GRIK2 expression in a transduced cell in the brain (e.g., the hippocampus) of the subject is reduced by 20% to 40%. In some embodiments, the level of GRIK2 expression in a transduced cell in the brain (e.g., the hippocampus) of the subject is reduced by 30%. In some embodiments, the level of GRIK2 is reduced (e.g., by at least 10-60%) for at least 28 days (e.g., at least 30 days, at least 60 days, at least 120 days, at least 365 days, or more). In some embodiments, the level of GRIK2 is reduced (e.g., by at least 10-60%) for at least 30 days. In some embodiments, the level of GRIK2 is reduced (e.g., by at least 10-60%) for at least 60 days. In some embodiments, the level of GRIK2 is reduced (e.g., by at least 10-60%) for at least 120 days. In some embodiments, the level of GRIK2 is reduced (e.g., by at least 10-60%) for at least 365 days. In some embodiments, the level of GRIK2 is reduced (e.g., by at least 10-60%) for 1-5 years, or for the lifetime of the subject. In some embodiments, the level of GRIK2 is reduced (e.g., by at least 10-60%) for at least 10 years or more, or for the lifetime of the subject. In some embodiments, the level of GRIK2 is reduced (e.g., by at least 10-60%) for at least 20 years or more, or for the lifetime of the subject.

Administration of the compositions described herein by the methods of the disclosure provides a reduction in the level of GluK2 expression in a transduced cell in the brain (e.g., the hippocampus) of the subject. Accordingly, the level of GluK2 expression in a transduced cell in the brain (e.g., the hippocampus) of the subject is reduced by at least 10%, at least at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or more, e.g., relative to a control AAV vector or relative to a cell in the brain (e.g., the hippocampus) of the subject that is not transduced. In some embodiments, the level of GluK2 expression in a transduced cell in the brain (e.g., the hippocampus) of the subject is reduced by 5% to 60% (e.g., 10% to 50%, 20% to 40%, or 30%). In some embodiments, the level of GluK2 expression in a transduced cell in the brain (e.g., the hippocampus) of the subject is reduced by 10% to 50%. In some embodiments, the level of GluK2 expression in a transduced cell in the brain (e.g., the hippocampus) of the subject is reduced by 20% to 40%. In some embodiments, the level of GluK2 expression in a transduced cell in the brain (e.g., the hippocampus) of the subject is reduced by 30%. In some embodiments, the level of GluK2 is reduced (e.g., by at least 10-60%) for at least 28 days (e.g., at least 30 days, at least 60 days, at least 120 days, at least 365 days, or more). In some embodiments, the level of GluK2 is reduced (e.g., by at least 10-60%) for at least 30 days. In some embodiments, the level of GluK2 is reduced (e.g., by at least 10-60%) for at least 60 days. In some embodiments, the level of GluK2 is reduced (e.g., by at least 10-60%) for at least 120 days. In some embodiments, the level of GluK2 is reduced (e.g., by at least 10-60%) for at least 365 days. In some embodiments, the level of GluK2 is reduced (e.g., by at least 10-60%) for 1-5 years, or for the lifetime of the subject. In some embodiments, the level of GluK2 is reduced (e.g., by at least 10-60%) for at least 10 years or more, or for the lifetime of the subject. In some embodiments, the level of GluK2 is reduced (e.g., by at least 10-60%) for at least 20 years or more, or for the lifetime of the subject.

Administration of the compositions described herein by the methods of the disclosure:
(a) reduces the number of seizures per day and/or reduces epileptiform discharges in the subject, for example, as measured by an electroencephalogram and standardized to seizure frequency per 30 days;
(b) improves the subject's measurements on routine laboratory parameters, such as hematology, biochemistry, coagulation, and urinalysis parameters, within at least 4 weeks, at least 5 weeks, at least 6 weeks, at least 7 weeks, at least 8 weeks, at least 3 months, at least 4 months, at least 5 months, at least 6 months, or at least 1 year after the method is performed;
(c) reduces frequency of interictal discharges, as measured by an electroencephalogram;
(d) reduces aberrant neurological behavior by the subject; and/I(e) produces no adverse effects after 4 weeks.

In some embodiments, the compositions described herein are administered once yearly. In some embodiments, the compositions described herein are administered once per lifetime.

### Measuring GRIK2 Inhibition

Upon administration, the inhibitory ribopolynucleotides or AAV vectors encoding the same of the disclosure are capable of inhibiting the expression of a GRIK2 mRNA, resulting in reduced levels (e.g., by at least 10-60% relative to a cell in the subject's hippocampus that is not transduced) of GRIK2 mRNA and GluK2 protein in a transduced cell in the subject's hippocampus. For example, the expression of GRIK2 is decreased in a first cell or group of cells in the subject's hippocampus as compared to a second cell or group of cells substantially identical to the first cell or group of cells but which has not or have not been so treated (control cell(s) not treated with an AAV vector or inhibitory ribopolynucleotide of the disclosure, or cell(s) in the hippocampus of the subject that are not transduced). The degree of decrease in the level of Grik2 mRNA or GluK2 protein may be expressed in terms of:

$$\frac{( mRNA in control cells ) - ( mRNA in treated cells )}{( mRNA in control cells )} \times 100 \%$$

A change in the level of expression of GRIK2 may be manifested by a decrease in the level of the GluK2 protein that is expressed by a cell or group of cells (e.g., the level of GluK2 protein expressed in a sample derived from a subject). As is explained above, for the assessment of GRIK2 mRNA suppression, the change in the level of GluK2 protein expression in a treated cell or group of cells may similarly be expressed as a percentage of the level of protein in a control cell or group of cells.

The level of GRIK2 mRNA expressed by a cell or group of cells may be determined using any method known in the art for assessing mRNA expression. For example, the level of expression GRIK2 mRNA in a sample may be determined by detecting a transcribed polynucleotide, or portion thereof, e.g., mRNA. RNA may be extracted from cells using RNA extraction techniques including, for example, using acid phenol/guanidine isothiocyanate extraction (RNAzol B; Biogenesis), RNEASY™ RNA preparation kits (Qiagen) or PAXgene (PreAnalytix, Switzerland). Typical assay formats utilizing ribonucleic acid hybridization include nuclear run-on assays, RT-pCR, RNase protection assays, northern blotting, in situ hybridization, and microarray analysis. Circulating mRNA may be detected using methods the described in PCT Publication WO2012/177906, the entire contents of which are hereby incorporated herein by reference. The level of expression of Grik2 mRNA may also be determined using a nucleic acid probe.

One method for the determination of mRNA levels involves contacting the extracted mRNA with a nucleic acid molecule that can hybridize to the Grik2 mRNA. The mRNA may be immobilized on a solid surface and contacted with a probe, for example by running the isolated mRNA on an agarose gel and transferring the mRNA from the gel to a membrane, such as nitrocellulose. The probe(s) may also be immobilized on a solid surface and the mRNA is contacted with the probe(s), for example, in an AFFYMETRIX gene chip array. Known mRNA detection methods in the art may be adapted for use in determining the level of Grik2 mRNA.

An alternative method for determining the level of expression of Grik2 mRNA in a sample involves the process of nucleic acid amplification and/or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known in the art. These detection schemes are especially useful for the detection of nucleic acid molecules if such molecules are present in very low numbers. In particular aspects of the disclosure, the level of expression of Grik2 mRNA can be determined by, e.g., quantitative fluorogenic RT-PCR (i.e., the TAQMAN™ System) or the DUAL-GLO® Luciferase assay.

The level of mRNA expression may also be assessed using real time quantitative PCR (qPCR). The use of this PCR method is described and exemplified in the Examples presented herein. Such methods can also be used for the detection of Grik2 nucleic acids.

Furthermore, the level of GluK2 protein produced by the expression the GRIK2gene may be determined using any method known in the art for the measurement of protein levels. Such methods include, for example, electrophoresis, capillary electrophoresis, chromatography, fluid or gel precipitin reactions, absorption spectroscopy, a colorimetric assays, spectrophotometric assays, flow cytometry, immunodiffusion (single or double), immunoelectrophoresis, western blotting, radioimmunoassay (RIA), enzyme-linked immunosorbent assays (ELISAs), immunofluorescent assays, electrochemiluminescence assays, and the like. Such assays can also be used for the detection of proteins indicative of the presence or replication of proteins produced by the gene of interest.

In addition, the assays described above may be utilized to determine whether a subject (e.g., a subject suffering from an epilepsy, such as, e.g., TLE) has responded to treatment using the compositions and methods disclosed herein. For example, hippocampal brain tissue from an epileptogenic brain hemisphere(s) can be obtained from the TLE-afflicted subject by way of a small biopsy prior to treatment with the compositions and methods disclosed herein and expression of GRIK2 mRNA or GluK2 protein may be assessed using the aforementioned assays. The subject may then be administered treatment according to the methods and compositions disclosed herein. Subsequent to the recovery of the patient following treatment (e.g., 30, 60, 90, or more days after treatment) with the disclosed methods and compositions, a second biopsy may be performed over the same brain regions assessed prior to treatment and levels of GRIK2 mRNA or GluK2 protein may again be assessed. A showing that the TLE-afflicted subject exhibits lower (e.g., by at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more (e.g., 100%)) levels of expression of GRIK2 mRNA or GluK2 protein would indicate that the subject was responsive to treatment.

Other methods that can be used to assess GRIK2 mRNA or GluK2 protein levels without requiring a biopsy are also envisioned.

GRIK2 mRNA or GluK2 protein levels in the treated subject may also be compared with expression of the same from one or more healthy control subjects. A showing that GRIK2 mRNA or GluK2 protein levels in the TLE-afflicted subject after treatment are statistically indistinguishable from levels of the same in one or more healthy control subjects would indicate that the subject is responsive or has responded to treatment. GRIK2 mRNA levels or GluK2 protein levels in the neuronal cells of a treated subject can also be compared to standard or reference levels of these analytes that are known to indicate the absence of a disease state.

### Routes of Administration

The compositions disclosed herein may be administered to a subject (e.g., a subject identified as having TLE) using standard methods. For example, the compositions disclosed herein can be administered by systemic administration, such as parenteral (e.g., intra-parenchymal) administration.

In particular, the inhibitory ribopolynucleotides and AAV vectors encoding the same may be administered locally to brain tissue of the subject, such as brain tissue determined to exhibit increased epileptiform activity. Local administration to the brain generally includes any method suitable for delivery of an inhibitory ribopolynucleotide or an AAV vector encoding the same to brain cells (e.g., neural cells), such that at least a portion of cells of a selected, synaptically connected cell population is contacted with the composition. Vectors may be delivered to any cells of the CNS, including neurons.

The vectors of the disclosure may be delivered by way of stereotactic injections or microinjections directly into the parenchyma or ventricles of the CNS. In a particular example, the vectors of the disclosure may be delivered directly to one or more epileptic foci in the brain of the subject. For example, the subject may be administered a vector of the disclosure by means of a stereotactic injection directly into one or both hemispheres of the allocortex (e.g., hippocampus) or neocortex (e.g., frontal lobe). In a particular example, the subject is administered a vector of the disclosure by means of a stereotactic injection directly into one or both hemispheres of the hippocampus. Alternatively, the vectors of the disclosure may be administered by intravenous injection, for example in the context of vectors that exhibit tropism for CNS tissues, including but not limited to an AAV described herein, such as, e.g., AAV2, AAV5, or AAV9.

To deliver a vector of the disclosure specifically to a particular region and to a particular population of CNS cells, the vector may be administered by stereotaxic microinjection. For example, subjects may have a stereotactic frame base surgically fixed in place (screwed into the skull). The brain with a stereotactic frame base (e.g., MRI compatible stereotactic frame base with fiducial markings) is imaged using high resolution MRI. The MRI images are then transferred to a computer which runs stereotactic software. A series of coronal, sagittal and axial images are used to determine the target injection site and trajectory of the cannula or injection needle used for injecting a composition of the disclosure into the brain. The software directly translates the trajectory into three-dimensional coordinates appropriate for the stereotactic frame. Holes are drilled above the entry site and the stereotactic apparatus is positioned with the injection needle implanted at the given depth. The composition (such as a composition disclosed herein) may be injected at the target sites. In the case that the composition includes an integrating vector, rather than producing viral particles, the spread of the vector is minor and mainly a function of passive diffusion from the site of injection. The degree of diffusion may be controlled by adjusting the ratio of vector to fluid carrier. This method is referred to as MRI-guided convection enhanced delivery (CED).

The AAV vector (e.g., an AAV9 vector) containing an inhibitor ribopolynucleotide (e.g., SEQ ID NOs: 2, 7, 8, 13, or 16, or variants thereof with at least 95% sequence identity thereto) can be administered to the subject in a single dose per hemisphere by advancing a needle through the region of the brain (e.g., the hippocampus) and injecting a volume of the composition containing the AAV vector at between 1-10 (e.g., between 1-10, between 2-9, between 3-8, between 4-7, or between 5-6) focal sites within the brain (e.g., the hippocampus). In some embodiments, the composition is administered to the subject at 5 or 6 focal sites within the brain (e.g., the hippocampus). The focal sites may be identified using an MRI or PET scan. The volume of the dose may be equally divided by the number of focal sites.

### Combination Therapy

The compositions disclosed herein may be administered to a subject in need thereof (e.g., a human subject) to treat an epilepsy (e.g., a TLE) in combination with one or more additional therapeutic modalities (e.g., 1, 2, 3, or more additional therapeutic modalities), including other therapeutic agents or physical interventions (e.g., rehabilitation therapy or surgical intervention). The two or more agents can be administered at the same time (e.g., administration of all agents occurs within 15 minutes, 10 minutes, 5 minutes, 2 minutes or less). The agents can also be administered simultaneously via co-formulation. The two or more agents can also be administered sequentially, such that the action of the two or more agents overlaps and their combined effect is such that the reduction in a symptom, or other parameter related to the disorder is greater than what would be observed with one agent or treatment delivered alone or in the absence of the other. The effect of the two or more treatments can be partially additive, wholly additive, or greater than additive (e.g., synergistic). The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 24 hours, or up to 7 days, 14 days, 21 days, or 30 days before or after the second therapeutic agent.

In cases in which the subject is diagnosed with or displaying one or more (e.g., two, three, four, or more) symptoms of epilepsy (e.g., a TLE), the second therapeutic agent may include one or more antiepileptic drug (AED) including, but not limited to valproate, lamotrigine, ethosuximide, topiramate, lacosamide, levetiracetam, clobazam, stiripentol, benzodiazepine, phenytoin, carbamazepine, primidone, phenobarbital, gabapentin, pregabalin, tiagabine, zonisamide, felbamate, and/or vigabatrin. Additional therapeutic modalities that can be administered together with the methods and compositions of the disclosure include vagus nerve stimulation, deep brain stimulation, transcranial magnetic stimulation, responsive neurostimulation, external trigeminus nerve stimulation, low glycemic index treatment, medium chain triglyceride diet, and ketogenic diet.

### Kits

The disclosure also provides a kit that includes a composition (e.g., an inhibitory ribopolynucleotide or a vector, such as a viral vector, e.g., an AAV vector, encoding the same) disclosed herein (e.g., an AAV9 vector containing an inhibitor ribopolynucleotide, such as, e.g., SEQ ID NOs: 2, 7, 8, 13, or 16, or variants thereof with at least 95% sequence identity thereto) that inhibits the expression of a GRIK2 gene in a subject (e.g., an inhibitory RNA targeting a GRIK2 mRNA) for use in the prevention or treatment of an epilepsy (e.g., a TLE, such as treatment-refractory TLE, or a focal epilepsy). The composition may be present in the kit in, e.g., a vial. The composition may be formulated for administration of the AAV vector to the subject intra-parenchymally, and may include an amount of the AAV vector of from about 1×10⁹ vg to about 1×10¹³ vg (e.g., about 1×10¹¹ vg/mL to about 1×10¹³ vg/mL), and, e.g., in a volume of about 3.0 mL or less (e.g., 1.8 mL or less). The kit can optionally include an agent or device for delivering the composition to the subject. In other examples, the kit may include one or more sterile applicators, such as syringes or needles. Further, the kit may optionally include other agents, e.g., anesthetics or antibiotics. The kit can also include a package insert that instructs a user of the kit, such as a physician, to perform the methods disclosed herein.

### Sequences

The foregoing sequences are represented as DNA (i.e., cDNA) sequences that can be incorporated into a vector of the disclosure. These sequences may also be represented as corresponding RNA sequences that are synthesized from the vector within the cell. One skilled in the art would understand that the cDNA sequence is equivalent to the mRNA sequence, except for the substitution of uridines with thymidines, and can be used for the same purpose herein, i.e., the generation of a polynucleotide for inhibiting the expression of Grik2 mRNA. In the case of DNA vectors (e.g., AAV), the polynucleotide containing the antisense nucleic acid is a DNA sequence. In the case of RNA vectors, the transgene incorporates the RNA equivalent of the antisense DNA sequences described herein.

TABLE 1
Sequence Listing

| SEQ ID | | |
|---|---|---|
| NO: | Description: | Sequence: |
| 1 | Construct A | CTGCGCGCTCGCTCGCTCACTGAGGCCGCCCGGGCAAAGCCCGGGCGTC |
| | | GGGCGACCTTTGGTCGCCCGGCCTCAGTGAGCGAGCGAGCGCGCAGAGA |
| | | GGGAGTGGCCAACTCCATCACTAGGGGTTCCTTGTAGTTAATGATTAACCC |
| | | GCCATGCTACTTATCTACCAGGGTAATGGGGATCCTCTAGAACTATAGCTA |
| | | GTCGACGACGGGGTGAACTACGCCTGAGGATCCGATCTTTTTCCCTCTGC |
| | | CAAAAATTATGGGGACATCATGAAGCCCCTTGAGCATCTGACTTCTGGCTA |
| | | ATAAAGGAAATTTATTTTCATTGCAATAGTGTGTTGGAATTTTTTGTGTCTCT |
| | | CACTCGGTTGATCTGAAGCAATTCGTTGAGTCTGCCTTCGCCCTTGCCCAC |
| | | TCTCTCACCTCCTCCCCCCGGCTCTGACTGACTTTACTCCCACAGGTGAGC |
| | | GGCGGATGCCCTTCTCCTCTGGCTGTAATTAGTCTTGGTTTACTTGTTTCTT |
| | | TTCTGTGGCTGTTGAAAGCCTTGAGGGGCTCTGGAGGGCCCTTTGTGTGG |
| | | GGGAGTGCTTGGGGGTGTTGTTGTGTGTGTGTTGGGGAGTCTTTGTGCTC |
| | | TTCTGCCTGTGCTGTGAGTCTGTGGTTGTTGGGCTTTGTGTCTCTCAGTGT |
| | | GTTAGGGGAGTTGCTGGGGTGTGCCCTTGTGTGGGGGGGCTGTAGGGGA |
| | | ACAAAGGCTGTTGTGGGTGTGTGTTGGGGGGGTGAGCAGGGGGTGTGGG |
| | | TTTTGTTGGCTGCAACCCCCCCTGCACCCCCCTCCCTAGTTGCTGAGCATG |
| | | CCTGCTTTGGTGTGGGCTCTTATGGGTTGGTTGGGCTTCTTGCTGGTGGG |
| | | GGTGGTGCAGGTGGGGGTGCTGGTGGGTGGGCTCCTTGGCTGGGAGGGC |
| | | TTGGGGAGGGGTTGTGCCCCTGAGTCTGTGCTGTTAGGTTGTAGCTCAGC |
| | | CATTGCCTTTTTTGTAGAGGGTCAGGGACTTCCTTTGTCCCAAATCTGTGTG |
| | | AGCTAAATCTGGGAGGTCTCTCACCCCCTCTAGTGGTTGGGTAAGTGTGTG |
| | | TCTGCAGGAAGGAAAGGGTGGGAGGGCCTTTTGTTTCTTCTCTTCCCCTTC |
| | | TCCCTCTCCAGCCTTGGGCTGTCTTGGGGGATGCTGCCTTTGGGGGGATG |
| | | GGCAGGGTGGGTTTGCTTCTGGTTGTGACTGTGCTCTAGAGCCTCTGCTAA |
| | | CCTGTTCTGCCTTCTTCTTTTTCCTACAGCTCCTGGGCAATTGCTGGTTATT |
| | | GTGCTGTCTCTATTTTGGCAAAGAATTCTCTGAATTTCGACCACCCATAATA |
| | | CCCATTACCCTGGTAGATATAGTCTATCCAGGTTGAGCATCCTGCTGGTGG |
| | | TTACAAGAAACTGTTTGAAACTGTGGAGGAACTGTCCTCGCCGCTCACAGC |
| | | TCATGTAACAGGCAGGATCCCCCTCTGGCTCACCGGCAGTCTCCTTCGATG |
| | | TGGGCCAGGACTCTTTGAAGTTGGATCTGAGCCATTTTACCACCTGTTTGA |
| | | TGGGCAAGCCCTCCTGCACAAGTTTGACTTTAAAGAAGGACATGTCACATA |
| | | CCACAGAAGGTTCATCCGCACTGATGOTTACGTACGGGCAATGACTGAGAA |
| | | AAGGATCGTCATAACAGAATTTGGCACCTGTGCTTTCCCAGATCCCTGCAA |
| | | GAATATATTTTCCAGGTTTTTTTCTTACTTTCGAGGAGTAGAGGTTACTGAC |
| | | AATTGCCCTTGTTAATGTCTACCCAGTGGGGGAAGATTACTACGCTTGCAC |
| | | AGAGACCAACTTTATTACAAAGATTAATCCAGAGACCTTGGAGACAATTAAG |
| | | CAGGTTGATCTTTGCAACTAAGTCTCTGTCAATGGGGCCACTGCTCACCCC |
| | | CACATTGAAAATGATGGAACCGTTTACAATATTGGTAATTGCTTTGGAAAAA |
| | | ATTTTTCAATTGCCTACAACATTGTAAAGATCCCACCACTGCAAGCAGACAA |
| | | GGAAGATCCAATAAGCAAGTCAGAGATCGTTGTACAATTCCCCTGCAGTGA |
| | | CCGATTCAAGCCATCTTACGTTCATAGTTTTGGTCTGACTCCCAACTATATC |
| | | GTTTTTGTGGAGACACCAGTCAAAATTAACCTGTTCAAGTTCCTTTCTTCAT |
| | | GGAGTCTTTGGGGAGCCAACTACATGGATTGTTTTGAGTCCAATGAAACCA |
| | | TGGGGTTTGGCTTCATATTGCTGACAAAAAAAGGAAAAAGTACCTCAATAAT |
| | | AAATACAGAACTTCTCCTTTCAACCTCTTCCATCACATCAACACCTATGAAG |
| | | ACAATGGGTTTCTGATTGTGGATCTCTGCTGCTGGAAAGGATTTGAGTTTGT |
| | | TTATAATTACTTATATTTAGCCAATTTACGTGAGAACTGGGAAGAGGTGAAA |
| | | AAAAATGCCAGAAAGGCTCCCCAACCTGAAGTTAGGAGATATGTACTTCCT |
| | | TTGAATATTGACAAGGCTGACACAGGCAAGAATTTAGTCAGCTCCCCAATA |
| | | CAACTGCCACTGCAATTCTGTGCAGTGACGAGACTATCTGGCTGGAGCCTG |
| | | AAGTTCTCTTTTCAGGGCCTCGTCAAGCATTTGAGTTTCCTCAAATCAATTA |
| | | CCAGAAGTATTGTGGGAAACCTTACACATATGCGTATGGACTTGGCTTGAA |
| | | TCACTTTGTTCCAGATAGGCTCTGTAAGCTGAATGTCAAAACTAAAGAAACT |
| | | TGGGTTTGGCAAGAGCCTGATTCATACCCATCAGAACCCATCTTTGTTTCTC |
| | | ACCCAGATGCCTTGGAAGAAGATGATGGTGTAGTTCTGAGTGTGGTGGTGA |
| | | GCCCAGGAGCAGGACAAAAGCCTGCTTATCTCCTGATTCTGAATGCCAAGG |
| | | ACTTAAGTGAAGTTGCCCGGGCTGAAGTGGAGATTAACATCCCTGTCACCT |
| | | TTCATGGACTGTTCAAAAAATCTTGAAAGTAGCTAGCGCATGGGGGGTTAA |
| | | TCATTAACTACAAGGAACCCCTAGTGATGGAGTTGGCCACTCCCTCTCTGC |
| | | GCGCTCGCTCGCTCACTGAGGCCGGGCGACCAAAGGTCGCCCGACGCCC |
| | | GGGCTTTGCCCGGGGGGCCTCAGTGAGCGAGCGAGCGCGCAG |
| 2 | Construct B | CTGCGCGCTCGCTCGCTCACTGAGGCCGCCCGGGCAAAGCCCGGGCGTC |
| | | GGGCGACCTTTGGTCGCCCGGCCTCAGTGAGCGAGCGAGCGCGCAGAGA |
| | | GGGAGTGGCCAACTCCATCACTAGGGGTTCCTTGTAGTTAATGATTAACCC |
| | | GCCATGCTACTTATCTACCAGGGTAATGGGGATCCTCTAGATCCGGTCGGG |
| | | CCCGCGGTACCGTCGAGAAGCTTGATGTGGGGGGAGCTTCGAAGGGGCG |
| | | GGCGCCCGTGGGGGGGGTCCTGAGTGGGGGGGGACCGGGGCCGGCAC |
| | | CTGGGTGAGGTTCTGCAGAGGGCCCTGCGTATGAGTGCAAGTGGGTTTTA |
| | | GGACCAGGATGAGGCGGGGTGGGGGTGCCTACCTGACGACCGACCCCGA |
| | | CCCACTGGACAAGCACCCAACCCCCATTCCCCAAATTGCGCATCCCCTATC |
| | | AGAGAGGGGGAGGGGAAACAGGATGCGGCGAGGCGCGTGCGCACTGCC |
| | | AGCTTCAGCACCGCGGACAGTGCCTTCGCCCCCGCCTGGCGGCGCGCGC |
| | | CACCGCCGCCTCAGCACTGAAGGCGCGCTGACGTCACTCGCCGGTCCCC |
| | | CGCAAACTCCCCTTCCCGGCCACCTTGGTCGCGTCCGCGCCGCCGCCGG |
| | | CCCAGCCGGACCGCACCACGCGAGGCGCGAGATAGGGGGGCACGGGCG |
| | | CGACCATCTGCGCTGCGGCGCCGGCGACTCAGCGCTGCCTCAGTCTGCG |
| | | GTGGGCAGCGGAGGAGTCGTGTCGTGCCTGAGAGCGCAGGGCGCGCCTA |
| | | GCCCGGGCTAGGTCGACTCGACTAGGGATAACAGGGTAATTGTTTGAATG |
| | | AGGCTTCAGTACTTTACAGAATCGTTGCCTGCACATCTTGGAAACACTTGCT |
| | | GGGATTACTTCTTCAGGTTAACCCAACAGAAGGCTAAAGAAGGTATATTGC |
| | | TGTTGACAGTGAGCGACTTCTCGATATGGAGAACCCAGGCCGTGAAGCCA |
| | | CAGATGGGCCTGGGTTTTATATCGAGCAGCTGCCTACTGCCTCGGACTTCA |
| | | AGGGGCTACTTTAGGAGCAATTATCTTGTTTACTAAAACTGAATACCTTGCT |
| | | ATCTCTTTGATACATTTTTACAAAGCTGAATTAAAATGGTATAAATTATCACG |
| | | GGATCCGAATTCACGTTTCCAGAACGTCTGTAGCTTTTCTCCTCCTTCCCTC |
| | | CATTTTCCTCTTGGTCTTACCTTTGGCCTAGTGGTTGGTGTAGTGATAATGT |
| | | AGCGAGATTTTCTGTAGAGCATTGCAGATGGACCGGGTTGCGAGGTATGA |
| | | GTAAACGGTCCATACGCAATGCTCCATGGAACGTCACGCAGCTTTCTACAG |
| | | CATGACAAGCTGCTGAGGCTTAAATCAGGATTTTCCTGTCTOTTTCTACAAA |
| | | ATCAAAATGAAAAAAGAGGGCTTTTTAGGCATCTCCGAGATTATGTGCTCGA |
| | | GGGGATCCGATCTTTTTCCCTCTGCCAAAAATTATGGGGACATCATGAAGC |
| | | CCCTTGAGCATCTGACTTCTGGCTAATAAAGGAAATTTATTTTCATTGCAAT |
| | | AGTGTGTTGGAATTTTTTGTGTCTCTCACTCGGCGGCCGCATAGTCTATCC |
| | | AGGTTGAGCATCCTGCTGGTGGTTACAAGAAACTGTTTGAAACTGTGGAGG |
| | | AACTGTCCTCGCCGCTCACAGCTCATGTAACAGGCAGGATCCCCCTCTGG |
| | | CTCACCGGCAGTCTCCTTCGATGTGGGCCAGGACTCTTTGAAGTTGGATCT |
| | | GAGCCATTTTACCACCTGTTTGATGGGCAAGCCCTCCTGCACAAGTTTGAC |
| | | TTTAAAGAAGGACATGTCACATACCACAGAAGGTTCATCCGCACTGATGCT |
| | | TACGTACGGGCAATGACTGAGAAAAGGATCGTCATAACAGAATTTGGCACC |
| | | TGTGCTTTCCCAGATCCCTGCAAGAATATATTTTCCAGGTTTTTTTCTTACTT |
| | | TCGAGGAGTAGAGGTTACTGACAATTGCCCTTGTTAATGTCTACCCAGTGG |
| | | GGGAAGATTACTACGCTTGCACAGAGACCAACTTTATTACAAAGATTAATCC |
| | | AGAGACCTTGGAGACAATTAAGCAGGTTGATCTTTGCAACTAAGTCTCTGT |
| | | CAATGGGGCCACTGCTCACCCCCACATTGAAAATGATGGAACCGTTTACAA |
| | | TATTGGTAATTGCTTTGGAAAAAATTTTTCAATTGCCTACAACATTGTAAAGA |
| | | TCCCACCACTGCAAGCAGACAAGGAAGATCCAATAAGCAAGTCAGAGATCG |
| | | TTGTACAATTCCCCTGCAGTGACCGATTCAAGCCATCTTACGTTCATAGTTT |
| | | TGGTCTGACTCCCAACTATATCGTTTTTGTGGAGACACCAGTCAAAATTAAC |
| | | CTGTTCAAGTTCCTTTCTTCATGGAGTCTTTGGGGAGCCAACTACATGGATT |
| | | GTTTTGAGTCCAATGAAACCATGGGGTTTGGCTTCATATTGCTGACAAAAAA |
| | | AGGAAAAAGTACCTCAATAATAAATACAGAACTTCTCCTTTCAACCTCTTCC |
| | | ATCACATCAACACCTATGAAGACAATGGGTTTCTGATTGTGGATCTCTGCTG |
| | | CTGGAAAGGATTTGAGTTTGTTTATAATTACTTATATTTAGCCAATTTACGTG |
| | | AGAACTGGGAAGAGGTGAAAAAAAATGCCAGAAAGGCTCCCCAACCTGAA |
| | | GTTAGGAGATATGTACTTCCTTTGAATATTGACAAGGCTGACACAGGCAAG |
| | | AATTTAGTCAGCTCCCCAATACAACTGCCACTGCAATTCTGTGCAGTGACG |
| | | AGACTATCTGGCTGGAGCCTGAAGTTCTCTTTTCAGGGCCTCGTCAAGCAT |
| | | TTGAGTTTCCTCAAATCAATTACCAGAAGTATTGTGGGAAACCTTACACATA |
| | | TGCGTATGGACTTGGCTTGAATCACTTTGTTCCAGATAGGCTCTGTAAGCT |
| | | GAATGTCAAAACTAAAGAAACTTGGGTTTGGCAAGAGCCTGATTCATACCC |
| | | ATCAGAACCCATCTTTGTTTCTCACCCAGATGCCTTGGAAGAAGATGATGG |
| | | TGTAGTTCTGAGTGTGGTGGTGAGCCCAGGAGCAGGACAAAAGCCTGCTT |
| | | ATCTCCTGATTCTGAATGCCAAGGACTTAAGTGAAGTTGCCCGGGCTGAAG |
| | | TGGAGATTAACATCCCTGTCACCTTTCATGGACTGTTCAAAAAATCTTGACC |
| | | GGCCGGCTAGCGAAGCAATTCGTTGATCTGAATTTCGACCACCCATAATAC |
| | | CCATTACCCTGGTAGATAAGTAGCATGGGGGGTTAATCATTAACTACAAGG |
| | | AACCCCTAGTGATGGAGTTGGCCACTCCCTCTCTGCGCGCTCGCTCGCTC |
| | | ACTGAGGCCGGGCGACCAAAGGTCGCCCGACGCCCGGGCTTTGCCCGGG |
| | | CGGCCTCAGTGAGCGAGCGAGCGCGCAG |
| 3 | Construct C | CTGCGCGCTCGCTCGCTCACTGAGGCCGCCCGGGCAAAGCCCGGGCGTC |
| | | GGGCGACCTTTGGTCGCCCGGCCTCAGTGAGCGAGCGAGCGCGCAGAGA |
| | | GGGAGTGGCCAACTCCATCACTAGGGGTTCCTTGTAGTTAATGATTAACCC |
| | | GCCATGCTACTTATCTACCAGGGTAATGGGGATCCTCTAGATCCGGTCGGG |
| | | CCCGCGGTACCGTCGAGAAGCTTGATGTGGGGGAGCTTCGAAGGGGCG |
| | | GGCGCCCGTGGGGGGGGTCCTGAGTGGGGGGGGACCGGGGCCGGCAC |
| | | CTGGGTGAGGTTCTGCAGAGGGCCCTGCGTATGAGTGCAAGTGGGTTTTA |
| | | GGACCAGGATGAGGCGGGGGGGGGTGCCTACCTGACGACCGACCCCGA |
| | | CCCACTGGACAAGCACCCAACCCCCATTCCCCAAATTGCGCATCCCCTATC |
| | | AGAGAGGGGGAGGGGAAACAGGATGCGGCGAGGCGCGTGCGCACTGCC |
| | | AGCTTCAGCACCGCGGACAGTGCCTTCGCCCCCGCCTGGCGGCGCGCGC |
| | | CACCGCCGCCTCAGCACTGAAGGCGCGCTGACGTCACTCGCCGGTCCCC |
| | | CGCAAACTCCCCTTCCCGGCCACCTTGGTCGCGTCCGCGCCGCCGCCGG |
| | | CCCAGCCGGACCGCACCACGCGAGGCGCGAGATAGGGGGGCACGGGCG |
| | | CGACCATCTGCGCTGCGGCGCCGGCGACTCAGCGCTGCCTCAGTCTGCC |
| | | AATTGCAGCGGAGGAGTCGTGTCGTGCCTGAGAGCGCAGGGCGCGCCTA |
| | | GCCCGGGCTAGGTCGACTCGACTAGGGATAACAGGGTAATTGTTTGAATG |
| | | AGGCTTCAGTACTTTACAGAATCGTTGCCTGCACATCTTGGAAACACTTGCT |
| | | GGGATTACTTCTTCAGGTTAACCCAACAGAAGGCTCGAGAAGGTATATTGC |
| | | TGTTGACAGTGAGCGCTAAAACAGGCATTAGCTATGGGTAGTGAAGCCACA |
| | | GATGCCCATAGCTAATGCCTGTTTTATTGCCTACTGCCTCGGAATTCAAGG |
| | | GGCTACTTTAGGAGCAATTATCTTGTTTACTAAAACTGAATACCTTGCTATCT |
| | | CTTTGATACATTTTTACAAAGCTGAATTAAAATGGTATAAATTATCACGGGAT |
| | | CCGATCTTTTTCCCTCTGCCAAAAATTATGGGGACATCATGAAGCCCCTTGA |
| | | GCATCTGACTTCTGGCTAATAAAGGAAATTTATTTTCATTGCAATAGTGTGTT |
| | | GGAATTTTTTGTGTCTCTCACTCGGCGGCCGCCCGAGTTTAATTGGTTTATA |
| | | GAACTCTTCAAGCTAGCGAAGCAATTCGTTGATCTGAATTTCGACCACCCAT |
| | | AATACCCATTACCCTGGTAGATAAGTAGCATGGGGGGTTAATCATTAACTAC |
| | | AAGGAACCCCTAGTGATGGAGTTGGCCACTCCCTCTCTGCGCGCTCGCTC |
| | | GCTCACTGAGGCCGGGCGACCAAAGGTCGCCCGACGCCCGGGCTTTGCC |
| | | CGGGGGGCCTCAGTGAGCGAGCGAGCGCGCAG |
| 5 | Construct E | CTGCGCGCTCGCTCGCTCACTGAGGCCGCCCGGGCAAAGCCCGGGCGTC |
| | | GGGCGACCTTTGGTCGCCCGGCCTCAGTGAGCGAGCGAGCGCGCAGAGA |
| | | GGGAGTGGCCAACTCCATCACTAGGGGTTCCTTGTAGTTAATGATTAACCC |
| | | GCCATGCTACTTATCTACCAGGGTAATGGGGATCCTCTAGATCCGGTCGGG |
| | | CCCGCGGTACCGTCGAGAAGCTTGATGTGGGGGAGCTTCGAAGGGGCG |
| | | GGCGCCCGTGGGGGGGGTCCTGAGTGGGGGGGGACCGGGGCCGGCAC |
| | | CTGGGTGAGGTTCTGCAGAGGGCCCTGCGTATGAGTGCAAGTGGGTTTTA |
| | | GGACCAGGATGAGGCGGGGTGGGGGTGCCTACCTGACGACCGACCCCGA |
| | | CCCACTGGACAAGCACCCAACCCCCATTCCCCAAATTGCGCATCCCCTATC |
| | | AGAGAGGGGGAGGGGAAACAGGATGCGGCGAGGCGCGTGCGCACTGCC |
| | | AGCTTCAGCACCGCGGACAGTGCCTTCGCCCCCGCCTGGCGGCGCGCGC |
| | | CACCGCCGCCTCAGCACTGAAGGCGCGCTGACGTCACTCGCCGGTCCCC |
| | | CGCAAACTCCCCTTCCCGGCCACCTTGGTCGCGTCCGCGCCGCCGCCGG |
| | | CCCAGCCGGACCGCACCACGCGAGGCGCGAGATAGGGGGGCACGGGCG |
| | | CGACCATCTGCGCTGCGGCGCCGGCGACTCAGCGCTGCCTCAGTCTGCC |
| | | AATTGCAGCGGAGGAGTCGTGTCGTGCCTGAGAGCGCAGGGCGCGCCTA |
| | | GCCCGGGCTAGGTCGACTCGACTAGGGATAACAGGGTAATTGTTTGAATG |
| | | AGGCTTCAGTACTTTACAGAATCGTTGCCTGCACATCTTGGAAACACTTGCT |
| | | GGGATTACTTCTTCAGGTTAACCCAACAGAAGGCTCGAGAAGGTATATTGC |
| | | TGTTGACAGTGAGCGACATGGGTTCTCCATATCGAGACTAGTGAAGCCACA |
| | | GATGGTCTCGATATGGAGAACCCATGCTGCCTACTGCCTCGGAATTCAAGG |
| | | GGCTACTTTAGGAGCAATTATCTTGTTTACTAAAACTGAATACCTTGCTATCT |
| | | CTTTGATACATTTTTACAAAGCTGAATTAAAATGGTATAAATTATCACGGGAT |
| | | CCGATCTTTTTCCCTCTGCCAAAAATTATGGGGACATCATGAAGCCCCTTGA |
| | | GCATCTGACTTCTGGCTAATAAAGGAAATTTATTTTCATTGCAATAGTGTGTT |
| | | GGAATTTTTTGTGTCTCTCACTCGGCGGCCGCCCGAGTTTAATTGGTTTATA |
| | | GAACTCTTCAAGCTAGCGAAGCAATTCGTTGATCTGAATTTCGACCACCCAT |
| | | AATACCCATTACCCTGGTAGATAAGTAGCATGGGGGGTTAATCATTAACTAC |
| | | AAGGAACCCCTAGTGATGGAGTTGGCCACTCCCTCTCTGCGCGCTCGCTC |
| | | GCTCACTGAGGCCGGGCGACCAAAGGTCGCCCGACGCCCGGGCTTTGCC |
| | | CGGGGGGCCTCAGTGAGCGAGCGAGCGCGCAG |
| 6 | Construct F | CTGCGCGCTCGCTCGCTCACTGAGGCCGCCCGGGCAAAGCCCGGGCGTC |
| | | GGGCGACCTTTGGTCGCCCGGCCTCAGTGAGCGAGCGAGCGCGCAGAGA |
| | | GGGAGTGGCCAACTCCATCACTAGGGGTTCCTTGTAGTTAATGATTAACCC |
| | | GCCATGCTACTTATCTACCAGGGTAATGGGGATCCTCTAGATCCGGTCGGG |
| | | CCCGCGGTACCGTCGAGAAGCTTGATGTGGGGGGAGCTTCGAAGGGGCG |
| | | GGCGCCCGTGGGGGGGGTCCTGAGTGGGGGGGGACCGGGGCCGGCAC |
| | | CTGGGTGAGGTTCTGCAGAGGGCCCTGCGTATGAGTGCAAGTGGGTTTTA |
| | | GGACCAGGATGAGGCGGGGTGGGGGTGCCTACCTGACGACCGACCCCGA |
| | | CCCACTGGACAAGCACCCAACCCCCATTCCCCAAATTGCGCATCCCCTATC |
| | | AGAGAGGGGGAGGGGAAACAGGATGCGGCGAGGCGCGTGCGCACTGCC |
| | | AGCTTCAGCACCGCGGACAGTGCCTTCGCCCCCGCCTGGCGGCGCGCGC |
| | | CACCGCCGCCTCAGCACTGAAGGCGCGCTGACGTCACTCGCCGGTCCCC |
| | | CGCAAACTCCCCTTCCCGGCCACCTTGGTCGCGTCCGCGCCGCCGCCGG |
| | | CCCAGCCGGACCGCACCACGCGAGGCGCGAGATAGGGGGGCACGGGCG |
| | | CGACCATCTGCGCTGCGGCGCCGGCGACTCAGCGCTGCCTCAGTCTGCG |
| | | GTGGGCAGCGGAGGAGTCGTGTCGTGCCTGAGAGCGCAGGGCGCGCCTA |
| | | GCCCGGGCTAGGTCGACTCGACTAGGGATAACAGGGTAATTGTTTGAATG |
| | | AGGCTTCAGTACTTTACAGAATCGTTGCCTGCACATCTTGGAAACACTTGCT |
| | | GGGATTACTTCTTCAGGTTAACCCAACAGAAGGCTAAAGAAGGTATATTGC |
| | | TGTTGACAGTGAGCGACGTCTCGATATGGAGAACCCATGCTGTGAAGCCA |
| | | CAGATGGGCATGGGTTTTATATCGAGACGCTGCCTACTGCCTCGGACTTCA |
| | | AGGGGCTACTTTAGGAGCAATTATCTTGTTTACTAAAACTGAATACCTTGCT |
| | | ATCTCTTTGATACATTTTTACAAAGCTGAATTAAAATGGTATAAATTATCACG |
| | | GGATCCGAATTCACGTTTCCAGAACGTCTGTAGCTTTTCTCCTCCTTCCCTC |
| | | CATTTTCCTCTTGGTCTTACCTTTGGCCTAGTGGTTGGTGTAGTGATAATGT |
| | | AGCGAGATTTTCTGCAGAGCATTGCAGATGGACTGGGTTGCGAGGTATGA |
| | | GTAAACAGTCCATACGCAATGCTCCGTGGAACGTCACGCAGCTTTCTACAG |
| | | CATGACAAGCTGCTGAGGCTTAAATCAGGATTTTCCTGTCTCTTTCTACAAA |
| | | ATCAAAATGAAAAAAGAGGGCTTTTTAGGCATCTCCGAGATTATGTGCTCGA |
| | | GGGGATCCGATCTTTTTCCCTCTGCCAAAAATTATGGGGACATCATGAAGC |
| | | CCCTTGAGCATCTGACTTCTGGCTAATAAAGGAAATTTATTTTCATTGCAAT |
| | | AGTGTGTTGGAATTTTTTGTGTCTCTCACTCGGCGGCCGCATAGTCTATCC |
| | | AGGTTGAGCATCCTGCTGGTGGTTACAAGAAACTGTTTGAAACTGTGGAGG |
| | | AACTGTCCTCGCCGCTCACAGCTCATGTAACAGGCAGGATCCCCCTCTGG |
| | | CTCACCGGCAGTCTCCTTCGATGTGGGCCAGGACTCTTTGAAGTTGGATCT |
| | | GAGCCATTTTACCACCTGTTTGATGGGCAAGCCCTCCTGCACAAGTTTGAC |
| | | TTTAAAGAAGGACATGTCACATACCACAGAAGGTTCATCCGCACTGATGCT |
| | | TACGTACGGGCAATGACTGAGAAAAGGATCGTCATAACAGAATTTGGCACC |
| | | TGTGCTTTCCCAGATCCCTGCAAGAATATATTTTCCAGGTTTTTTTCTTACTT |
| | | TCGAGGAGTAGAGGTTACTGACAATTGCCCTTGTTAATGTCTACCCAGTGG |
| | | GGGAAGATTACTACGCTTGCACAGAGACCAACTTTATTACAAAGATTAATCC |
| | | AGAGACCTTGGAGACAATTAAGCAGGTTGATCTTTGCAACTAAGTCTCTGT |
| | | CAATGGGGCCACTGCTCACCCCCACATTGAAAATGATGGAACCGTTTACAA |
| | | TATTGGTAATTGCTTTGGAAAAAATTTTTCAATTGCCTACAACATTGTAAAGA |
| | | TCCCACCACTGCAAGCAGACAAGGAAGATCCAATAAGCAAGTCAGAGATCG |
| | | TTGTACAATTCCCCTGCAGTGACCGATTCAAGCCATCTTACGTTCATAGTTT |
| | | TGGTCTGACTCCCAACTATATCGTTTTTGTGGAGACACCAGTCAAAATTAAC |
| | | CTGTTCAAGTTCCTTTCTTCATGGAGTCTTTGGGGAGCCAACTACATGGATT |
| | | GTTTTGAGTCCAATGAAACCATGGGGTTTGGCTTCATATTGCTGACAAAAAA |
| | | AGGAAAAAGTACCTCAATAATAAATACAGAACTTCTCCTTTCAACCTCTTCC |
| | | ATCACATCAACACCTATGAAGACAATGGGTTTCTGATTGTGGATCTCTGCTG |
| | | CTGGAAAGGATTTGAGTTTGTTTATAATTACTTATATTTAGCCAATTTACGTG |
| | | AGAACTGGGAAGAGGTGAAAAAAAATGCCAGAAAGGCTCCCCAACCTGAA |
| | | GTTAGGAGATATGTACTTCCTTTGAATATTGACAAGGCTGACACAGGCAAG |
| | | AATTTAGTCAGCTCCCCAATACAACTGCCACTGCAATTCTGTGCAGTGACG |
| | | AGACTATCTGGCTGGAGCCTGAAGTTCTCTTTTCAGGGCCTCGTCAAGCAT |
| | | TTGAGTTTCCTCAAATCAATTACCAGAAGTATTGTGGGAAACCTTACACATA |
| | | TGCGTATGGACTTGGCTTGAATCACTTTGTTCCAGATAGGCTCTGTAAGCT |
| | | GAATGTCAAAACTAAAGAAACTTGGGTTTGGCAAGAGCCTGATTCATACCC |
| | | ATCAGAACCCATCTTTGTTTCTCACCCAGATGCCTTGGAAGAAGATGATGG |
| | | TGTAGTTCTGAGTGTGGTGGTGAGCCCAGGAGCAGGACAAAAGCCTGCTT |
| | | ATCTCCTGATTCTGAATGCCAAGGACTTAAGTGAAGTTGCCCGGGCTGAAG |
| | | TGGAGATTAACATCCCTGTCACCTTTCATGGACTGTTCAAAAAATCTTGACC |
| | | GGCCGGCTAGCGAAGCAATTCGTTGATCTGAATTTCGACCACCCATAATAC |
| | | CCATTACCCTGGTAGATAAGTAGCATGGCGGGTTAATCATTAACTACAAGG |
| | | AACCCCTAGTGATGGAGTTGGCCACTCCCTCTCTGCGCGCTCGCTCGCTC |
| | | ACTGAGGCCGGGCGACCAAAGGTCGCCCGACGCCCGGGCTTTGCCCGGG |
| | | CGGCCTCAGTGAGCGAGCGAGCGCGCAG |
| 7 | Construct G | CTGCGCGCTCGCTCGCTCACTGAGGCCGCCCGGGCAAAGCCCGGGCGTC |
| | | GGGCGACCTTTGGTCGCCCGGCCTCAGTGAGCGAGCGAGCGCGCAGAGA |
| | | GGGAGTGGCCAACTCCATCACTAGGGGTTCCTTGTAGTTAATGATTAACCC |
| | | GCCATGCTACTTATCTACCAGGGTAATGGGGATCCTCTAGATCCGGTCGGG |
| | | CCCGCGGTACCGTCGAGAAGCTTGATGTGGGGGGAGCTTCGAAGGGGCG |
| | | GGCGCCCGTGGGGGGGGTCCTGAGTGGGGGGGGACCGGGGCCGGCAC |
| | | CTGGGTGAGGTTCTGCAGAGGGCCCTGCGTATGAGTGCAAGTGGGTTTTA |
| | | GGACCAGGATGAGGGGGGTGGGGGTGCCTACCTGACGACCGACCCCGA |
| | | CCCACTGGACAAGCACCCAACCCCCATTCCCCAAATTGCGCATCCCCTATC |
| | | AGAGAGGGGGAGGGGAAACAGGATGCGGCGAGGCGCGTGCGCACTGCC |
| | | AGCTTCAGCACCGCGGACAGTGCCTTCGCCCCCGCCTGGCGGCGCGCGC |
| | | CACCGCCGCCTCAGCACTGAAGGCGCGCTGACGTCACTCGCCGGTCCCC |
| | | CGCAAACTCCCCTTCCCGGCCACCTTGGTCGCGTCCGCGCCGCCGCCGG |
| | | CCCAGCCGGACCGCACCACGCGAGGCGCGAGATAGGGGGGCACGGGCG |
| | | CGACCATCTGCGCTGCGGCGCCGGCGACTCAGCGCTGCCTCAGTCTGCG |
| | | GTGGGCAGCGGAGGAGTCGTGTCGTGCCTGAGAGCGCAGGGCGCGCCTA |
| | | GCCCGGGCTAGGTCGACTCGACTAGGGATAACAGGGTAATTGTTTGAATG |
| | | AGGCTTCAGTACTTTACAGAATCGTTGCCTGCACATCTTGGAAACACTTGCT |
| | | GGGATTACTTCTTCAGGTTAACCCAACAGAAGGCTAAAGAAGGTATATTGC |
| | | TGTTGACAGTGAGCGACTTCTCGATATGGAGAACCCAGGCCGTGAAGCCA |
| | | CAGATGGGCCTGGGTTTTATATCGAGCAGCTGCCTACTGCCTCGGACTTCA |
| | | AGGGGCTACTTTAGGAGCAATTATCTTGTTTACTAAAACTGAATACCTTGCT |
| | | ATCTCTTTGATACATTTTTACAAAGCTGAATTAAAATGGTATAAATTATCACG |
| | | GGATCCgatctttttccctctgccaaaaattatggggacatcatgaagccccttgagcatctgacttc |
| | | tggctaataaaggaaatttattttcattgcaatagtgtgttggaattttttgtgtctctcactcgGCG |
| | | GCCGCATAGTCTATCCAGGTTGAGCATCCTGCTGGTGGTTACAAGAAACTGTTTGAAACTGT |
| | | GGAGGAACTGTCCTCGCCGCTCACAGCTCATGTAACAGGCAGGATCCCCC |
| | | TCTGGCTCACCGGCAGTCTCCTTCGATGTGGGCCAGGACTCTTTGAAGTTG |
| | | GATCTGAGCCATTTTACCACCTGTTTGATGGGCAAGCCCTCCTGCACAAGT |
| | | TTGACTTTAAAGAAGGACATGTCACATACCACAGAAGGTTCATCCGCACTG |
| | | ATGCTTACGTACGGGCAATGACTGAGAAAAGGATCGTCATAACAGAATTTG |
| | | GCACCTGTGCTTTCCCAGATCCCTGCAAGAATATATTTTCCAGGTTTTTTTC |
| | | TTACTTTCGAGGAGTAGAGGTTACTGACAATTGCCCTTGTTAATGTCTACCC |
| | | AGTGGGGGAAGATTACTACGCTTGCACAGAGACCAACTTTATTACAAAGAT |
| | | TAATCCAGAGACCTTGGAGACAATTAAGCAGGTTGATCTTTGCAACTAAGTC |
| | | TCTGTCAATGGGGCCACTGCTCACCCCCACATTGAAAATGATGGAACCGTT |
| | | TACAATATTGGTAATTGCTTTGGAAAAAATTTTTCAATTGCCTACAACATTGT |
| | | AAAGATCCCACCACTGCAAGCAGACAAGGAAGATCCAATAAGCAAGTCAGA |
| | | GATCGTTGTACAATTCCCCTGCAGTGACCGATTCAAGCCATCTTACGTTCAT |
| | | AGTTTTGGTCTGACTCCCAACTATATCGTTTTTGTGGAGACACCAGTCAAAA |
| | | TTAACCTGTTCAAGTTCCTTTCTTCATGGAGTCTTTGGGGAGCCAACTACAT |
| | | GGATTGTTTTGAGTCCAATGAAACCATGGGGTTTGGCTTCATATTGCTGACA |
| | | AAAAAAGGAAAAAGTACCTCAATAATAAATACAGAACTTCTCCTTTCAACCT |
| | | CTTCCATCACATCAACACCTATGAAGACAATGGGTTTCTGATTGTGGATCTC |
| | | TGCTGCTGGAAAGGATTTGAGTTTGTTTATAATTACTTATATTTAGCCAATTT |
| | | ACGTGAGAACTGGGAAGAGGTGAAAAAAAATGCCAGAAAGGCTCCCCAAC |
| | | CTGAAGTTAGGAGATATGTACTTCCTTTGAATATTGACAAGGCTGACACAG |
| | | GCAAGAATTTAGTCAGCTCCCCAATACAACTGCCACTGCAATTCTGTGCAG |
| | | TGACGAGACTATCTGGCTGGAGCCTGAAGTTCTCTTTTCAGGGCCTCGTCA |
| | | AGCATTTGAGTTTCCTCAAATCAATTACCAGAAGTATTGTGGGAAACCTTAC |
| | | ACATATGCGTATGGACTTGGCTTGAATCACTTTGTTCCAGATAGGCTCTGTA |
| | | AGCTGAATGTCAAAACTAAAGAAACTTGGGTTTGGCAAGAGCCTGATTCAT |
| | | ACCCATCAGAACCCATCTTTGTTTCTCACCCAGATGCCTTGGAAGAAGATG |
| | | ATGGTGTAGTTCTGAGTGTGGTGGTGAGCCCAGGAGCAGGACAAAAGCCT |
| | | GCTTATCTCCTGATTCTGAATGCCAAGGACTTAAGTGAAGTTGCCCGGGCT |
| | | GAAGTGGAGATTAACATCCCTGTCACCTTTCATGGACTGTTCAAAAAATCTT |
| | | GACCGGCCGGCTAGCGAAGCAATTCGTTGATCTGAATTTCGACCACCCATA |
| | | ATACCCATTACCCTGGTAGATAAGTAGCATGGGGGGTTAATCATTAACTACA |
| | | AGGAACCCCTAGTGATGGAGTTGGCCACTCCCTCTCTGCGCGCTCGCTCG |
| | | CTCACTGAGGCCGGGCGACCAAAGGTCGCCCGACGCCCGGGCTTTGCCC |
| | | GGGCGGCCTCAGTGAGCGAGCGAGCGCGCAG |
| 8 | Construct H | CTGCGCGCTCGCTCGCTCACTGAGGCCGCCCGGGCAAAGCCCGGGCGTC |
| | | GGGCGACCTTTGGTCGCCCGGCCTCAGTGAGCGAGCGAGCGCGCAGAGA |
| | | GGGAGTGGCCAACTCCATCACTAGGGGTTCCTTGTAGTTAATGATTAACCC |
| | | GCCATGCTACTTATCTACCAGGGTAATGGGGATCCTCTAGATCCGGTCGGG |
| | | CCCGCGGTACCGTCGAGAAGCTTGATGTGGGGGAGCTTCGAAGGGGCG |
| | | GGCGCCCGTGGGGGGGGTCCTGAGTGGGGGGGGACCGGGGCCGGCAC |
| | | CTGGGTGAGGTTCTGCAGAGGGCCCTGCGTATGAGTGCAAGTGGGTTTTA |
| | | GGACCAGGATGAGGCGGGGTGGGGGTGCCTACCTGACGACCGACCCCGA |
| | | CCCACTGGACAAGCACCCAACCCCCATTCCCCAAATTGCGCATCCCCTATC |
| | | AGAGAGGGGGAGGGGAAACAGGATGCGGCGAGGCGCGTGCGCACTGCC |
| | | AGCTTCAGCACCGCGGACAGTGCCTTCGCCCCCGCCTGGCGGCGCGCGC |
| | | CACCGCCGCCTCAGCACTGAAGGCGCGCTGACGTCACTCGCCGGTCCCC |
| | | CGCAAACTCCCCTTCCCGGCCACCTTGGTCGCGTCCGCGCCGCCGCCGG |
| | | CCCAGCCGGACCGCACCACGCGAGGCGCGAGATAGGGGGGCACGGGCG |
| | | CGACCATCTGCGCTGCGGCGCCGGCGACTCAGCGCTGCCTCAGTCTGCG |
| | | GTGGGCAGCGGAGGAGTCGTGTCGTGCCTGAGAGCGCAGGGCGCGCCTG |
| | | AATTCACGTTTCCAGAACGTCTGTAGCTTTTCTCCTCCTTCCCTCCATTTTC |
| | | CTCTTGGTCTTACCTTTGGCCTAGTGGTTGGTGTAGTGATAATGTAGCGAG |
| | | ATTTTCTGTAGAGCATTGCAGATGGACCGGGTTGCGAGGTATGAGTAAACG |
| | | GTCCATACGCAATGCTCCATGGAACGTCACGCAGCTTTCTACAGCATGACA |
| | | AGCTGCTGAGGCTTAAATCAGGATTTTCCTGTCTCTTTCTACAAAATCAAAA |
| | | TGAAAAAAGAGGGCTTTTTAGGCATCTCCGAGATTATGTGCTCGAGGGGAT |
| | | CCgatctttttccctctgccaaaaattatggggacatcatgaagccccttgagcatctgacttctggc |
| | | taataaaggaaatttattttcattgcaatagtgtgttggaattttttgtgtctctcactcgGCGGCCG |
| | | CATAGTCTATCCAGGTTGAGCATCCTGCTGGTGGTTACAAGAAACTGTTTGAAACTGTGGAGG |
| | | AACTGTCCTCGCCGCTCACAGCTCATGTAACAGGCAGGATCCCCCTCTGG |
| | | CTCACCGGCAGTCTCCTTCGATGTGGGCCAGGACTCTTTGAAGTTGGATCT |
| | | GAGCCATTTTACCACCTGTTTGATGGGCAAGCCCTCCTGCACAAGTTTGAC |
| | | TTTAAAGAAGGACATGTCACATACCACAGAAGGTTCATCCGCACTGATGCT |
| | | TACGTACGGGCAATGACTGAGAAAAGGATCGTCATAACAGAATTTGGCACC |
| | | TGTGCTTTCCCAGATCCCTGCAAGAATATATTTTCCAGGTTTTTTTCTTACTT |
| | | TCGAGGAGTAGAGGTTACTGACAATTGCCCTTGTTAATGTCTACCCAGTGG |
| | | GGGAAGATTACTACGCTTGCACAGAGACCAACTTTATTACAAAGATTAATCC |
| | | AGAGACCTTGGAGACAATTAAGCAGGTTGATCTTTGCAACTAAGTCTCTGT |
| | | CAATGGGGCCACTGCTCACCCCCACATTGAAAATGATGGAACCGTTTACAA |
| | | TATTGGTAATTGCTTTGGAAAAAATTTTTCAATTGCCTACAACATTGTAAAGA |
| | | TCCCACCACTGCAAGCAGACAAGGAAGATCCAATAAGCAAGTCAGAGATCG |
| | | TTGTACAATTCCCCTGCAGTGACCGATTCAAGCCATCTTACGTTCATAGTTT |
| | | TGGTCTGACTCCCAACTATATCGTTTTTGTGGAGACACCAGTCAAAATTAAC |
| | | CTGTTCAAGTTCCTTTCTTCATGGAGTCTTTGGGGAGCCAACTACATGGATT |
| | | GTTTTGAGTCCAATGAAACCATGGGGTTTGGCTTCATATTGCTGACAAAAAA |
| | | AGGAAAAAGTACCTCAATAATAAATACAGAACTTCTCCTTTCAACCTCTTCC |
| | | ATCACATCAACACCTATGAAGACAATGGGTTTCTGATTGTGGATCTCTGCTG |
| | | CTGGAAAGGATTTGAGTTTGTTTATAATTACTTATATTTAGCCAATTTACGTG |
| | | AGAACTGGGAAGAGGTGAAAAAAAATGCCAGAAAGGCTCCCCAACCTGAA |
| | | GTTAGGAGATATGTACTTCCTTTGAATATTGACAAGGCTGACACAGGCAAG |
| | | AATTTAGTCAGCTCCCCAATACAACTGCCACTGCAATTCTGTGCAGTGACG |
| | | AGACTATCTGGCTGGAGCCTGAAGTTCTCTTTTCAGGGCCTCGTCAAGCAT |
| | | TTGAGTTTCCTCAAATCAATTACCAGAAGTATTGTGGGAAACCTTACACATA |
| | | TGCGTATGGACTTGGCTTGAATCACTTTGTTCCAGATAGGCTCTGTAAGCT |
| | | GAATGTCAAAACTAAAGAAACTTGGGTTTGGCAAGAGCCTGATTCATACCC |
| | | ATCAGAACCCATCTTTGTTTCTCACCCAGATGCCTTGGAAGAAGATGATGG |
| | | TGTAGTTCTGAGTGTGGTGGTGAGCCCAGGAGCAGGACAAAAGCCTGCTT |
| | | ATCTCCTGATTCTGAATGCCAAGGACTTAAGTGAAGTTGCCCGGGCTGAAG |
| | | TGGAGATTAACATCCCTGTCACCTTTCATGGACTGTTCAAAAAATCTTGACC |
| | | GGCCGGCTAGCGAAGCAATTCGTTGATCTGAATTTCGACCACCCATAATAC |
| | | CCATTACCCTGGTAGATAAGTAGCATGGGGGGTTAATCATTAACTACAAGG |
| | | AACCCCTAGTGATGGAGTTGGCCACTCCCTCTCTGCGCGCTCGCTCGCTC |
| | | ACTGAGGCCGGGCGACCAAAGGTCGCCCGACGCCCGGGCTTTGCCCGGG |
| | | CGGCCTCAGTGAGCGAGCGAGCGCGCAG |
| 9 | Construct I | GTTTGAATGAGGCTTCAGTACTTTACAGAATCGTTGCCTGCACATCTTGGAA |
| | | ACACTTGCTGGGATTACTTCTTCAGGTTAACCCAACAGAAGGCTAAAGAAG |
| | | GTATATTGCTGTTGACAGTGAGCGACTTCTCGATATGGAGAACCCAGGCCG |
| | | TGAAGCCACAGATGGGCCTGGGTTTTATATCGAGCAGCTGCCTACTGCCTC |
| | | GGACTTCAAGGGGCTACTTTAGGAGCAATTATCTTGTTTACTAAAACTGAAT |
| | | ACCTTGCTATCTCTTTGATACATTTTTACAAAGCTGAATTAAAATGGTATAAA |
| | | TTA |
| 10 | Construct J | ACGTTTCCAGAACGTCTGTAGCTTTTCTCCTCCTTCCCTCCATTTTCCTCTT |
| | | GGTCTTACCTTTGGCCTAGTGGTTGGTGTAGTGATAATGTAGCGAGATTTT |
| | | CTGCAGAGCATTGCAGATGGACTGGGTTGCGAGGTATGAGTAAACAGTCC |
| | | ATACGCAATGCTCCGTGGAACGTCACGCAGCTTTCTACAGCATGACAAGCT |
| | | GCTGAGGCTTAAATCAGGATTTTCCTGTCTOTTTCTACAAAATCAAAATGAA |
| | | AAAAGAGGGCTTTTTAGGCATCTCCGAGATTATGTG |
| 11 | Construct K | TCTGCCGCGGAAAGGGGAGAAGTGTGGGCTCCTCCGAGTCGGGGGGGGA |
| | | CTGGGACAGCACAGTCGGCTGAGCGCAGCGCCCCCGCCCTGCCCGCCAC |
| | | GCGGCGAAGACGCCTGAGCGTTCGCGCCCCTCGGGCGAGGACCCCACGC |
| | | AAGCCCGAGCCGGTCCCGACCCTGGCCCCGACGCTCGCCGCCCGCCCCA |
| | | GCCCTGAGGGCCCCTCGACGTTTATCTACAACACTCTGATTTAATGTCTATA |
| | | CAATCAGAGCATTGCAGATGGACTGCGAGAGGCGCCTCCGCCGCTCCTTT |
| | | CTCATGGAAATGGCCCGCGAGCCCGTCCGGCCCAGCGCCCCTCCCGCGG |
| | | GAGGAAGGCGAGCCCGGCCCCCGGCGGCCATTCGCGCCGCGGACAAATC |
| | | CGGCGAACAATGCGCCCGCCCAGAGTGCGGCCCAGCTGCCGGGCCGGG |
| | | GATCTGGCCGCGGGACACAAAGGGGCCCGCACGCCTCTGGCGT |
| 12 | Construct L | CTGCGCGCTCGCTCGCTCACTGAGGCCGCCCGGGCAAAGCCCGGGCGTC |
| | | GGGCGACCTTTGGTCGCCCGGCCTCAGTGAGCGAGCGAGCGCGCAGAGA |
| | | GGGAGTGGCCAACTCCATCACTAGGGGTTCCTTGTAGTTAATGATTAACCC |
| | | GCCATGCTACTTATCTACCAGGGTAATGGGGATCCTCTAGATCCGGTCGGG |
| | | CCCGCGGTACCGTCGAGAAGCTTGATGTGGGCGGAGCTTCGAAGGGGCG |
| | | GGCGCCCGTGGGGGGGGTCCTGAGTGGGGGGGGACCGGGGCCGGCAC |
| | | CTGGGTGAGGTTCTGCAGAGGGCCCTGCGTATGAGTGCAAGTGGGTTTTA |
| | | GGACCAGGATGAGGCGGGGTGGGGGTGCCTACCTGACGACCGACCCCGA |
| | | CCCACTGGACAAGCACCCAACCCCCATTCCCCAAATTGCGCATCCCCTATC |
| | | AGAGAGGGGGAGGGGAAACAGGATGCGGCGAGGCGCGTGCGCACTGCC |
| | | AGCTTCAGCACCGCGGACAGTGCCTTCGCCCCCGCCTGGCGGCGCGCGC |
| | | CACCGCCGCCTCAGCACTGAAGGCGCGCTGACGTCACTCGCCGGTCCCC |
| | | CGCAAACTCCCCTTCCCGGCCACCTTGGTCGCGTCCGCGCCGCCGCCGG |
| | | CCCAGCCGGACCGCACCACGCGAGGCGCGAGATAGGGGGGCACGGGCG |
| | | CGACCATCTGCGCTGCGGCGCCGGCGACTCAGCGCTGCCTCAGTCTGCG |
| | | GTGGGCAGCGGAGGAGTCGTGTCGTGCCTGAGAGCGCAGGGCGCGCCTA |
| | | GCCCGGGCTAGGTCGACTCGACTAGGGATAACAGGGTAATTGTTTGAATG |
| | | AGGCTTCAGTACTTTACAGAATCGTTGCCTGCACATCTTGGAAACACTTGCT |
| | | GGGATTACTTCTTCAGGTTAACCCAACAGAAGGCTAAAGAAGGTATATTGC |
| | | TGTTGACAGTGAGCGACGTCTCGATATGGAGAACCCATGCTGTGAAGCCA |
| | | CAGATGGGCATGGGTTTTATATCGAGACGCTGCCTACTGCCTCGGACTTCA |
| | | AGGGGCTACTTTAGGAGCAATTATCTTGTTTACTAAAACTGAATACCTTGCT |
| | | ATCTCTTTGATACATTTTTACAAAGCTGAATTAAAATGGTATAAATTATCACG |
| | | GGATCCCTGTGCCTTCTAGTTGCCAGCCATCTGTTGTTTGCCCCTCCCCCG |
| | | TGCCTTCCTTGACCCTGGAAGGTGCCACTCCCACTGTCCTTTCCTAATAAA |
| | | ATGAGGAAATTGCATCGCATTGTCTGAGTAGGTGTCATTCTATTCTGGGGG |
| | | GTGGGGTGGGGCAGGACAGCAAGGGGGAGGATTGGGAAGACAATAGCAG |
| | | GCATGCTGGGGAGGCGGCCGCCCGAGTTTAATTGGTTTATAGAACTCTTCA |
| | | AGCTAGCACTAGTGAAGCAATTCGTTGCATTATGGCCTTAGGTCACTTCATC |
| | | TCCATGGGGTTCTTCTTCTGATTTTCTAGAAAATGAGATGGGGGTGCAGAG |
| | | AGCTTCCTCAGTGACCTGCCCAGGGTCACATCAGAAATGTCAGAGCTAGAA |
| | | CTTGAACTCAGATTACTAATCTTAAATTCCATGCCTTGGGGGCATGCAAGTA |
| | | CGATATACAGAAGGAGTGAACTCATTAGGGCAGATGACCAATGAGTTTAGG |
| | | AAAGAAGAGTCCAGGGCAGGGTACATCTACACCACCCGCCCAGCCCTGGG |
| | | TGAGTCCAGCCACGTTCACCTCATTATAGTTGCCTCTCTCCAGTCCTACCTT |
| | | GACGGGAAGCACAAGCAGAAACTGGGACAGGAGCCCCAGGAGACCAAAT |
| | | CTTCATGGTCCCTCTGGGAGGATGGGTGGGGAGAGCTGTGGCAGAGGCC |
| | | TCAGGAGGGGCCCTGCTGCTCAGTGGTGACAGATAGGGGTGAGAAAGCA |
| | | GACAGAGTCATTCCGTCAGCATTCTGGGTCTGTTTGGTACTTCTTCTCACG |
| | | CTAAGGTGGCGGTGTGATATGCACAATGGCTAAAAAGCAGGGAGAGCTGG |
| | | AAAGAAACAAGGACAGAGACAGAGGCCAAGTCAACCAGACCAATTCCCAG |
| | | AGGAAGCAAAGAAACCATTACAGAGACTACAAGGGGGAAGGGAAGGAGAG |
| | | ATGAATTAGCTTCCCCTGTAAACCTTAGAACCCAGCTGTTGCCAGGGCAAC |
| | | GGGGCAATACCTGTCTCTTCAGAGGAGATGAAGTTGCCAGGGTAACTACAT |
| | | CCTGTCTTTCTCAAGGACCATCCCAGAATGTGGCACCCACTAGCCGTTACC |
| | | ATAGCAACTGCCTCTTTGCCCCACTTAATCCCATCCCGTCTGTTAAAAGGG |
| | | CCCTATAGTTGGAGGTGGGGGAGGTAGGAAGAGCGATGATCACTTGTGGA |
| | | CTAAGTTTGTTCGCATCCCCTTCTCCAACCCCCTCAGTACATCACCCTGGG |
| | | GGAACAGGGTCCACTTGCTCCTGGGCCCACACAGTCCTGCAGTATTGTGT |
| | | ATATAAGGCCAGGGCAAAGAGGAGCAGGTTTTAAAGTGAAAGGCAGGCAG |
| | | GTGTTGGGGAGGCAGTTACCGGGGCAACGGGAACAGGGCGTTTCGGAGG |
| | | TGGTTGCCATGGGGACCTGGATGCTGACGAAGGCTCGCGAGGCTGTGAG |
| | | CAGCCACAGTGCCCTGCTCAGAAGCCCCAAGCTCGTCAGTCAAGCCGGTT |
| | | CTCCGTTTGCACTCAGGAGCACGGGCAGGCGAGTGGCCCCTAGTTCTGGG |
| | | GGCAGCGAATTCCAATTGGCGCGCCTAGCCCGGGCTAGGTCGACTCTGCC |
| | | GCGGAAAGGGGAGAAGTGTGGGCTCCTCCGAGTCGGGGGGGGACTGGGA |
| | | CAGCACAGTCGGCTGAGCGCAGCGCCCCCGCCCTGCCCGCCACGCGGCG |
| | | AAGACGCCTGAGCGTTCGCGCCCCTCGGGCGAGGACCCCACGCAAGCCC |
| | | GAGCCGGTCCCGACCCTGGCCCCGACGCTCGCCGCCCGCCCCAGCCCTG |
| | | AGGGCCCCTCGACGTTTATCTACAACACTCTGATTTAATGTCTATACAATCA |
| | | GAGCATTGCAGATGGACTGCGAGAGGCGCCTCCGCCGCTCCTTTCTCATG |
| | | GAAATGGCCCGCGAGCCCGTCCGGCCCAGCGCCCCTCCCGCGGGAGGAA |
| | | GGCGAGCCCGGCCCCCGGCGGCCATTCGCGCCGCGGACAAATCCGGCGA |
| | | ACAATGCGCCCGCCCAGAGTGCGGCCCAGCTGCCGGGCCGGGGATCTGG |
| | | CCGCGGGACACAAAGGGGCCCGCACGCCTCTGGCGTACCGGTCTCGAGG |
| | | GATCCGATCTTTTTCCCTCTGCCAAAAATTATGGGGACATCATGAAGCCCCT |
| | | TGAGCATCTGACTTCTGGCTAATAAAGGAAATTTATTTTCATTGCAATAGTG |
| | | TGTTGGAATTTTTTGTGTCTCTCACTCGGCGGCCGCCCGAGTTTAATTGGTT |
| | | TATAGAACTCTTCAAGCTAGCGAAGCAATTCGTTGATCTGAATTTCGACCAC |
| | | CCATAATACCCATTACCCTGGTAGATAAGTAGCATGGGGGGTTAATCATTAA |
| | | CTACAAGGAACCCCTAGTGATGGAGTTGGCCACTCCCTCTCTGCGCGCTC |
| | | GCTCGCTCACTGAGGCCGGGCGACCAAAGGTCGCCCGACGCCCGGGCTT |
| | | TGCCCGGGGGGCCTCAGTGAGCGAGCGAGCGCGCAG |
| 13 | miR38R | GTTTGAATGAGGCTTCAGTACTTTACAGAATCGTTGCCTGCACATCTTGGAA |
| | and | ACACTTGCTGGGATTACTTCTTCAGGTTAACCCAACAGAAGGCTAAAGAAG |
| | miR3bR | GTATATTGCTGTTGACAGTGAGCGACTTCTCGATATGGAGAACCCAGGCCG |
| | | TGAAGCCACAGATGGGCCTGGGTTTTATATCGAGCAGCTGCCTACTGCCTC |
| | | GGACTTCAAGGGGCTACTTTAGGAGCAATTATCTTGTTTACTAAAACTGAAT |
| | | ACCTTGCTATCTCTTTGATACATTTTTACAAAGCTGAATTAAAATGGTATAAA |
| | | TTATCACGGGATCCGAATTCACGTTTCCAGAACGTCTGTAGCTTTTCTCCTC |
| | | CTTCCCTCCATTTTCCTCTTGGTCTTACCTTTGGCCTAGTGGTTGGTGTAGT |
| | | GATAATGTAGCGAGATTTTCTGTAGAGCATTGCAGATGGACCGGGTTGCGA |
| | | GGTATGAGTAAACGGTCCATACGCAATGCTCCATGGAACGTCACGCAGCTT |
| | | TCTACAGCATGACAAGCTGCTGAGGCTTAAATCAGGATTTTCCTGTCTCTTT |
| | | CTACAAAATCAAAATGAAAAAAGAGGGCTTTTTAGGCATCTCCGAGATTATG |
| | | TG |
| 14 | miR3bR | ACGTTTCCAGAACGTCTGTAGCTTTTCTCCTCCTTCCCTCCATTTTCCTCTT |
| | | GGTCTTACCTTTGGCCTAGTGGTTGGTGTAGTGATAATGTAGCGAGATTTT |
| | | CTGTAGAGCATTGCAGATGGACCGGGTTGCGAGGTATGAGTAAACGGTCC |
| | | ATACGCAATGCTCCATGGAACGTCACGCAGCTTTCTACAGCATGACAAGCT |
| | | GCTGAGGCTTAAATCAGGATTTTCCTGTCTCTTTCTACAAAATCAAAATGAA |
| | | AAAAGAGGGCTTTTTAGGCATCTCCGAGATTATGTG |
| 15 | miR38R | GTTTGAATGAGGCTTCAGTACTTTACAGAATCGTTGCCTGCACATCTTGGAA |
| | | ACACTTGCTGGGATTACTTCTTCAGGTTAACCCAACAGAAGGCTAAAGAAG |
| | | GTATATTGCTGTTGACAGTGAGCGACTTCTCGATATGGAGAACCCAGGCCG |
| | | TGAAGCCACAGATGGGCCTGGGTTTTATATCGAGCAGCTGCCTACTGCCTC |
| | | GGACTTCAAGGGGCTACTTTAGGAGCAATTATCTTGTTTACTAAAACTGAAT |
| | | ACCTTGCTATCTCTTTGATACATTTTTACAAAGCTGAATTAAAATGGTATAAA |
| | | TTA |
| 16 | miR3bR | CTGCGCGCTCGCTCGCTCACTGAGGCCGCCCGGGCAAAGCCCGGGCGTC |
| | and | GGGCGACCTTTGGTCGCCCGGCCTCAGTGAGCGAGCGAGCGCGCAGAGA |
| | miR38R | GGGAGTGGCCAACTCCATCACTAGGGGTTCCTTGTAGTTAATGATTAACCC |
| | | GCCATGCTACTTATCTACCAGGGTAATGGGGATCCTCTAGATCCGGTCGGG |
| | | CCCGCGGTACCGTCGAGAAGCTTGATGTGGGCGGAGCTTCGAAGGGGCG |
| | | GGCGCCCGTGGGGGGGGTCCTGAGTGGGGGGGGACCGGGGCCGGCAC |
| | | CTGGGTGAGGTTCTGCAGAGGGCCCTGCGTATGAGTGCAAGTGGGTTTTA |
| | | GGACCAGGATGAGGGGGGGTGGGGGTGCCTACCTGACGACCGACCCCGA |
| | | CCCACTGGACAAGCACCCAACCCCCATTCCCCAAATTGCGCATCCCCTATC |
| | | AGAGAGGGGGAGGGGAAACAGGATGCGGCGAGGCGCGTGCGCACTGCC |
| | | AGCTTCAGCACCGCGGACAGTGCCTTCGCCCCCGCCTGGCGGCGCGCGC |
| | | CACCGCCGCCTCAGCACTGAAGGCGCGCTGACGTCACTCGCCGGTCCCC |
| | | CGCAAACTCCCCTTCCCGGCCACCTTGGTCGCGTCCGCGCCGCCGCCGG |
| | | CCCAGCCGGACCGCACCACGCGAGGCGCGAGATAGGGGGGCACGGGCG |
| | | CGACCATCTGCGCTGCGGCGCCGGCGACTCAGCGCTGCCTCAGTCTGCG |
| | | GTGGGCAGCGGAGGAGTCGTGTCGTGCCTGAGAGCGCAGGGCGCGCCTA |
| | | GCCCGGGCTAGGTCGACTCGACTAGGGATAACAGGGTAATTACGTTTCCA |
| | | GAACGTCTGTAGCTTTTCTCCTCCTTCCCTCCATTTTCCTCTTGGTCTTACC |
| | | TTTGGCCTAGTGGTTGGTGTAGTGATAATGTAGCGAGATTTTCTGTAGAGC |
| | | ATTGCAGATGGACCGGGTTGCGAGGTATGAGTAAACGGTCCATACGCAAT |
| | | GCTCCATGGAACGTCACGCAGCTTTCTACAGCATGACAAGCTGCTGAGGCT |
| | | TAAATCAGGATTTTCCTGTCTCTTTCTACAAAATCAAAATGAAAAAAGAGGG |
| | | CTTTTTAGGCATCTCCGAGATTATGTGTCACGGGATCCGAATTCGTTTGAAT |
| | | GAGGCTTCAGTACTTTACAGAATCGTTGCCTGCACATCTTGGAAACACTTG |
| | | CTGGGATTACTTCTTCAGGTTAACCCAACAGAAGGCTAAAGAAGGTATATT |
| | | GCTGTTGACAGTGAGCGACTTCTCGATATGGAGAACCCAGGCCGTGAAGC |
| | | CACAGATGGGCCTGGGTTTTATATCGAGCAGCTGCCTACTGCCTCGGACTT |
| | | CAAGGGGCTACTTTAGGAGCAATTATCTTGTTTACTAAAACTGAATACCTTG |
| | | CTATCTCTTTGATACATTTTTACAAAGCTGAATTAAAATGGTATAAATTACTC |
| | | GAGGGGATCCGATCTTTTTCCCTCTGCCAAAAATTATGGGGACATCATGAA |
| | | GCCCCTTGAGCATCTGACTTCTGGCTAATAAAGGAAATTTATTTTCATTGCA |
| | | ATAGTGTGTTGGAATTTTTTGTGTCTCTCACTCGGCGGCCGCATAGTCTATC |
| | | CAGGTTGAGCATCCTGCTGGTGGTTACAAGAAACTGTTTGAAACTGTGGAG |
| | | GAACTGTCCTCGCCGCTCACAGCTCATGTAACAGGCAGGATCCCCCTCTG |
| | | GCTCACCGGCAGTCTCCTTCGATGTGGGCCAGGACTCTTTGAAGTTGGATC |
| | | TGAGCCATTTTACCACCTGTTTGATGGGCAAGCCCTCCTGCACAAGTTTGA |
| | | CTTTAAAGAAGGACATGTCACATACCACAGAAGGTTCATCCGCACTGATGC |
| | | TTACGTACGGGCAATGACTGAGAAAAGGATCGTCATAACAGAATTTGGCAC |
| | | CTGTGCTTTCCCAGATCCCTGCAAGAATATATTTTCCAGGTTTTTTTCTTACT |
| | | TTCGAGGAGTAGAGGTTACTGACAATTGCCCTTGTTAATGTCTACCCAGTG |
| | | GGGGAAGATTACTACGCTTGCACAGAGACCAACTTTATTACAAAGATTAATC |
| | | CAGAGACCTTGGAGACAATTAAGCAGGTTGATCTTTGCAACTAAGTCTCTG |
| | | TCAATGGGGCCACTGCTCACCCCCACATTGAAAATGATGGAACCGTTTACA |
| | | ATATTGGTAATTGCTTTGGAAAAAATTTTTCAATTGCCTACAACATTGTAAAG |
| | | ATCCCACCACTGCAAGCAGACAAGGAAGATCCAATAAGCAAGTCAGAGATC |
| | | GTTGTACAATTCCCCTGCAGTGACCGATTCAAGCCATCTTACGTTCATAGTT |
| | | TTGGTCTGACTCCCAACTATATCGTTTTTGTGGAGACACCAGTCAAAATTAA |
| | | CCTGTTCAAGTTCCTTTCTTCATGGAGTCTTTGGGGAGCCAACTACATGGAT |
| | | TGTTTTGAGTCCAATGAAACCATGGGGTTTGGCTTCATATTGCTGACAAAAA |
| | | AAGGAAAAAGTACCTCAATAATAAATACAGAACTTCTCCTTTCAACCTCTTC |
| | | CATCACATCAACACCTATGAAGACAATGGGTTTCTGATTGTGGATCTCTGCT |
| | | GCTGGAAAGGATTTGAGTTTGTTTATAATTACTTATATTTAGCCAATTTACGT |
| | | GAGAACTGGGAAGAGGTGAAAAAAAATGCCAGAAAGGCTCCCCAACCTGA |
| | | AGTTAGGAGATATGTACTTCCTTTGAATATTGACAAGGCTGACACAGGCAA |
| | | GAATTTAGTCAGCTCCCCAATACAACTGCCACTGCAATTCTGTGCAGTGAC |
| | | GAGACTATCTGGCTGGAGCCTGAAGTTCTCTTTTCAGGGCCTCGTCAAGCA |
| | | TTTGAGTTTCCTCAAATCAATTACCAGAAGTATTGTGGGAAACCTTACACAT |
| | | ATGCGTATGGACTTGGCTTGAATCACTTTGTTCCAGATAGGCTCTGTAAGCT |
| | | GAATGTCAAAACTAAAGAAACTTGGGTTTGGCAAGAGCCTGATTCATACCC |
| | | ATCAGAACCCATCTTTGTTTCTCACCCAGATGCCTTGGAAGAAGATGATGG |
| | | TGTAGTTCTGAGTGTGGTGGTGAGCCCAGGAGCAGGACAAAAGCCTGCTT |
| | | ATCTCCTGATTCTGAATGCCAAGGACTTAAGTGAAGTTGCCCGGGCTGAAG |
| | | TGGAGATTAACATCCCTGTCACCTTTCATGGACTGTTCAAAAAATCTTGACC |
| | | GGCCGGCTAGCGAAGCAATTCGTTGATCTGAATTTCGACCACCCATAATAC |
| | | CCATTACCCTGGTAGATAAGTAGCATGGGGGGTTAATCATTAACTACAAGG |
| | | AACCCCTAGTGATGGAGTTGGCCACTCCCTCTCTGCGCGCTCGCTCGCTC |
| | | ACTGAGGCCGGGCGACCAAAGGTCGCCCGACGCCCGGGCTTTGCCCGGG |
| | | CGGCCTCAGTGAGCGAGCGAGCGCGCAG |

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1: Pharmacological Activity of Construct B in Pilocarpine Mice

### Mouse Studies

Examples 1-3 describe studies conducted in pilocarpine mice. Table 2 includes a summary of each study design.

TABLE 2
In Vivo Studies

| | Treatment | | | |
|---|---|---|---|---|
| | duration | Test item/ | | |
| | (Days/Weeks/ | Dose levels in | Sex and No. | |
| Study Description | Months) | vg/hippocampus | per group | Endpoints |
| PD marker: in-vivo | 6 weeks | Construct A, | 5 males/ | Epileptiform |
| hyperlocomotion in | | Construct B | group | Discharges (EEG |
| disease model | | 5.0 × 10⁸ vg/ | | recording) |
| | | hippocampus | | Locomotor |
| | | | | activity |
| Study in pilocarpine mice | 6 weeks | Construct A, | 10 males/ | Epileptiform |
| to investigate reduction of | | Construct B | group | Discharges (EEG |
| seizure activity using | | 5.0 × 10⁹ vg/ | | recording) |
| EEG recording for | | hippocampus | | Locomotor |
| Construct B vs Construct A | | | | activity |
| at a single dose of 1.0 × 10¹⁰ | | | | |
| Study in pilocarpine | 6 weeks | Construct A | 10 males/ | Epileptiform |
| mouse model to | | 5.0 × 10⁹ vg/ | group | Discharges (EEG |
| investigate reduction of | | hippocampus | | recording) |
| epileptiform activity in 3 | | Construct B | | Locomotor |
| different doses of | | 5.0 × 10⁷, 5.0 × | | activity |
| Construct B compared to | | 10⁸ and 5.0 × 10⁹ | | |
| Construct A using the | | vg/hippocampus | | |
| hyperlocomotion test and | | | | |
| EEG recording | | | | |
| Mouse Study to | 6 weeks | Diluent | 8 males/ | Epileptiform |
| investigate reduction of | | Construct B | group | Discharges (EEG |
| epileptiform activity in 3 | | 5.0 × 10⁸, 5.0 × | | recording) |
| different doses of | | 10⁹ and 5.0 × 10¹⁰ | | Locomotor |
| Construct B toxicology | | vg/hippocampus | | activity |
| material compared to | | | | |
| Construct C and diluent | | | | |
| using EEG recordings | | | | |
| PK/PD study of wild type | 6 months | Diluent | 9 males/ | AAV9 vector |
| Swiss mice | | Construct B | group | distribution |
| | | 5.0 × 10¹⁰ vg/ | | (qPCR) |
| | | hippocampus | | Mature miRNAs |
| | | | | (SL RT-PCR for |
| | | | | miR38R and |
| | | | | miR3bR) |
| | | | | GRIK2 mRNA |
| | | | | (RT-PCR) |

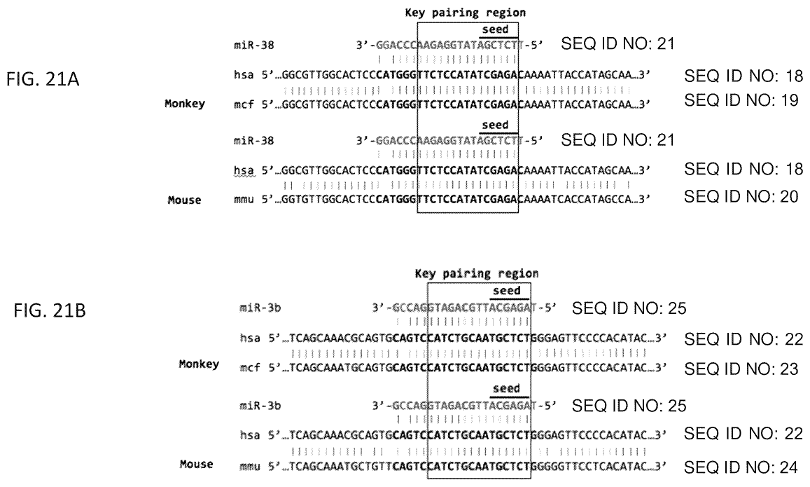

Construct B is a non-replicating, recombinant AAV9 vector that delivers a genome encoding 2 different concatenated gene-suppressing engineered miRNAs targeting the GRIK2 mRNA (FIG. 21). The activity of Construct B was characterized using the pilocarpine mouse model, which is preferred over the kainate model because it recapitulates human disease more accurately. For example, the pilocarpine mouse model displays major network reorganization and mossy fiber sprouting following an acute induction of status epilepticus, leading to the formation of powerful recurrent excitatory circuits between DGCs and resulting in chronic epileptiform activity (Vigier 2021). In addition, the model produces spontaneous seizures and shows resistance to classical anti-epileptic drugs in the therapeutic range, which is also seen in patients with TILE (Jones 2002).

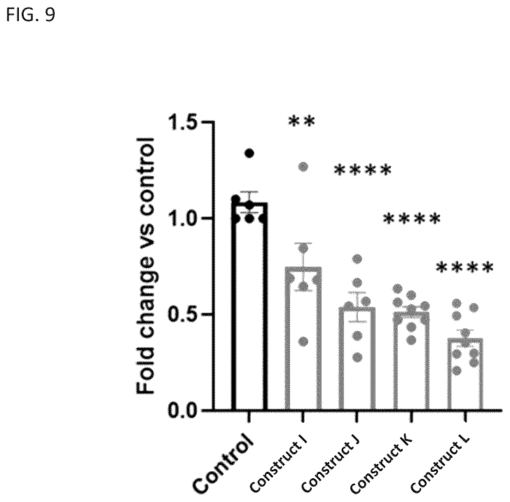

The effect of microRNA constructs miR3b and miR38 individually and in combination was assessed for their effect on GluK2 protein expression in primary mouse cortical neurons cultures using Western blot analysis. The transduction of mouse cortical neurons was performed with control vectors (Construct D) and test items (Construct I, Construct J, Construct K, and Construct L) (Table 3). Construct J, Construct K, and Construct L presented a significant reduction in GluK2 expression (FIG. 9). In conclusion, expression of miR3b (Construct J and Construct K) or miR38 (Construct 1) alone or in combination (Construct L) leads to a significant decrease of GluK2 in mouse cortical neurons.

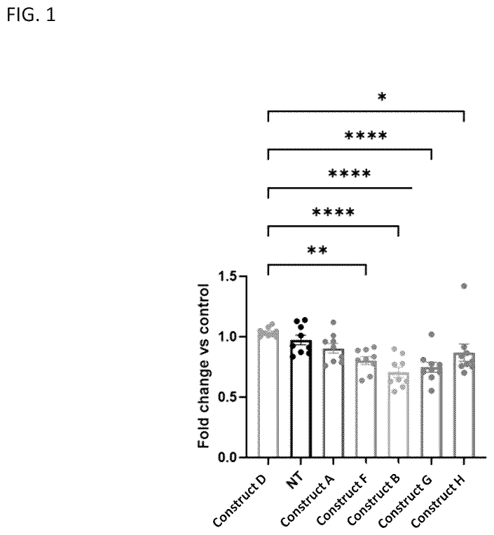

With additional experiments, 2 constructs were added under the single control of the human synapsin 1 promoter (Construct F). The sequences of miR38 and miR3b were modified to optimize the guide passenger ratio and the constructs were renamed miR38R and miR3bR, thereby obtaining the Construct B candidate. The constructs used in this study are summarized in Table 2. Transduction of mouse cortical neurons with Construct B, Construct G, and Construct H led to a significant reduction of GluK2 protein expression level, as measured by Western blot, comparable to what was observed with Construct F (FIG. 1). The candidate construct, Construct B, with optimized packaging and guide/passenger ratio, showed a significant effect in decreasing GluK2 protein level.

TABLE 3
Constructs

| Virus Name | Genome Content |
|---|---|
| Construct A | Vector control with no coding sequence |
| Construct B | hSyn.amiR38v2R3.E-miR-30.amiR3bR2.E-miR-218-1 |
| Construct C | hSyn.amiR30.hu1010 |
| Construct D | hSyn.GFP |
| Construct E | hSyn.miR30.amiR38 |
| Construct F | hSyn.amiR38v2.E-miR-30.amiR3b.E-miR-218-1 |
| Construct G | hSyn.amiR38v2R3.E-miR-30.stuffer |
| Construct H | hSyn.amiR3bR2.E-miR-218-1.stuffer |
| Construct I | hSyn.amiR38v2.E-miR-30 |
| Construct J | hSyn.amiR3b.E-miR-218-1 |
| Construct K | hSyn.amiR3b.E-miR-124-3 |
| Construct L | hSyn.amiR38v2.E-miR-30.CamKII.amiR3b.E-miR-124-3 |
| Construct M | hSyn.GFP.miR30.scr2 |

Expression of miR3bR or miR38R in a single construct under the control of the human synapsin 1 promoter leads to a significant decrease of GluK2 in mouse cortical neurons cultured in vitro. The miRNAs were optimized to favor the expression of the guide miRNA strand over the passenger. Construct B achieves robust AAV9 packaging and guide passenger ratios for constructs expressing miR3bR and miR38R. Two miRNAs were included in the construct to improve GluK2 knockdown by targeting GRIK2 mRNA at two different locations. The construct promotes consistent and significant reduction of GluK2 protein expression in mouse cortical neurons cultured in vitro (FIG. 1).

### Example 2: Construct B Dose-Response in Pilocarpine Mice

### PK/PD Safety Study in Wild-Type Swiss Mice

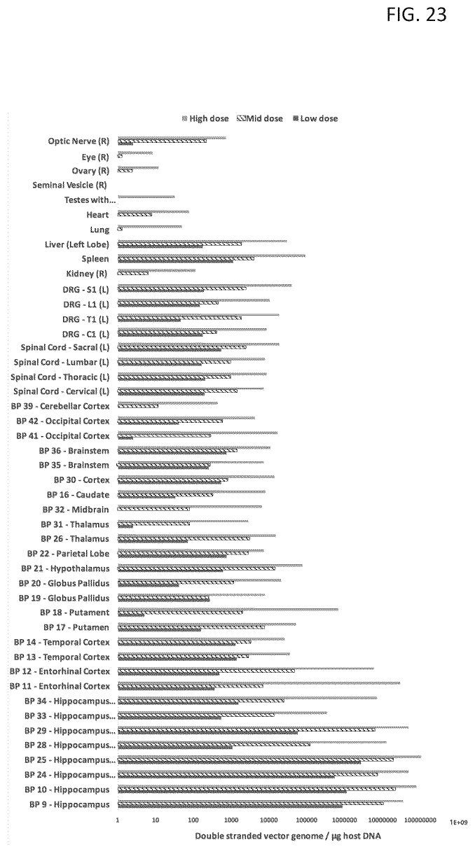

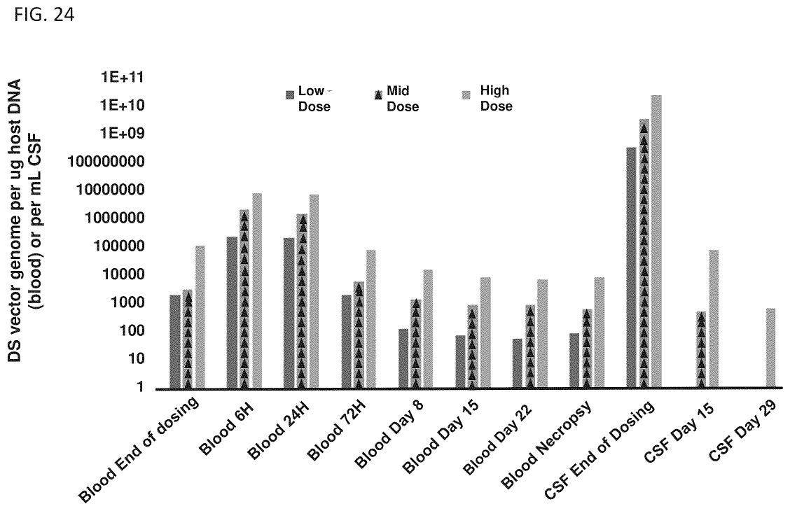

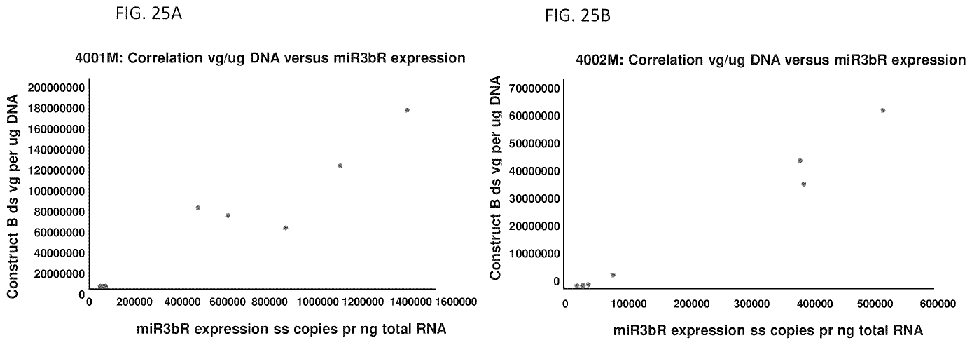

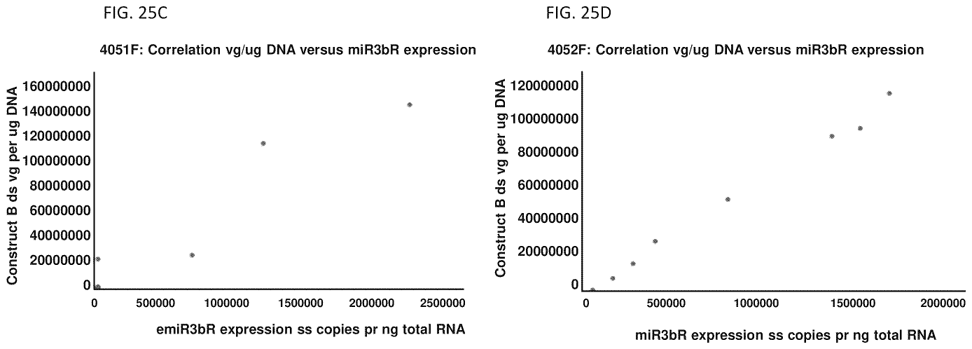

This study examined the biodistribution and tolerability of three doses of Construct B (10.0×10⁹, 1.0×10¹⁰, 1 0.0×10¹¹ vg/brain with 20 mice per group) after bilateral infusion to the dentate gyrus (DG). The mice received a total volume of 4 mL per animal and the doses covered the range from minimal efficacious dose to 100-fold excess. Animals were sacrificed after 42 days and extensively sampled for biodistribution (10 mice/group) and histopathology (remaining 10 mice per group) (Table 4). Brain and peripheral organ biodistribution of Construct B and mature miRNA expression/GRIK2 mRNA levels were assessed (FIG. 23 and FIG. 24).

TABLE 4
Serum Neutralizing Antibody (NAb)
Titers Pre-Screening and On-Study

| Animal/Group | Pre-Screening | Pre-Dose | Day 15 | Necropsy |
|---|---|---|---|---|
| 1001/Group 1 | <10 | <4 | <4 | Insufficient |
| | | | | sample |
| 1501/Group 1 | <10 | <4 | <4 | Insufficient |
| | | | | sample |
| 2001/Group 2 | <10 | <4 | 180 | 60 |
| 2501/Group 2 | Negative | <4 | 20 | 4 |
| 3001/Group 3 | <10 | <10 | 540 | 320 |
| 3002/Group 3 | <10 | <4 | 320 | 192 |
| 3501/Group 3 | Negative | <10 | 180 | 60 |
| 3502/Group 3 | Negative | <10 | 180 | 180 |
| 4001/Group 4 | Negative | <10 | 180 | 20 |
| 4002/Group 4 | Negative | Negative | 60 | 192 |
| 4501/Group 4 | <10 | <10 | 960 | 192 |
| 4502/Group 4 | <10 | <4 | 540 | 960 |

A dose-response study with Construct B in the pilocarpine-induced mouse model of chronic epilepsy with bilateral intra-hippocampal administration at dose levels of 1×10⁸, 1×10⁹, and 1×10¹⁰ vg/brain was performed. EEGs were selected as the primary endpoint and locomotor activity was selected as the secondary endpoint to assess the efficacy of the drug treatment in the pilocarpine mouse model. EEGs are used in clinic to monitor epileptic events in patients, and were used to quantify the efficacy of the drug treatment since they provided a primary readout to quantify spontaneous recurrent seizures. EEG recordings were performed using telemetry recordings via implanted EEG electrodes. To increase the robustness of the results of the drug treatment efficacy, we included the monitoring of locomotion as a secondary endpoint. Mice treated with pilocarpine show increased locomotor activity (Müller 2009; Smolensky 2019), likely due to increased extracellular concentrations of various mediators, such as dopamine opioids and glutamate. Hyperlocomotion (quantified as distance traveled in centimeters), is therefore increased in mice that have seizures.

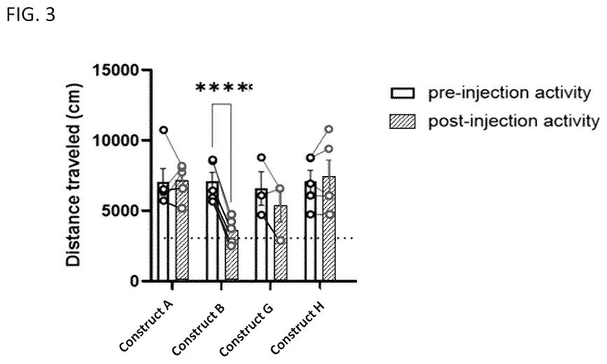

For this study, we used constructs expressing either 1 or 2 miRNAs, optimized for a high expression of guide versus passenger (Constructs B, G, and H). Construct A was used as a control. Results showed that the concatemer construct, Construct B was effective in significantly reducing the distance traveled by the pilocarpine model in vivo at 1×10⁹ vg/brain dose (FIG. 3). Single miRNA constructs with stuffer, Construct G to Construct H, did not significantly reduced the distance traveled. The concatemer design found in Construct B with 1 promoter driving expression of 2 miRNAs appears to be more effective than the single designs found in Construct G and Construct H with 1 promoter driving expression of 1 miRNA.

In vivo data with Construct B show improvement in the pathophysiological (epileptogenic hippocampus, mossy fibers sprouting, interneuron cell death and progressive hippocampal sclerosis) and behavioral (daily spontaneous recurrent seizures and significant cognitive decline) hallmarks of TLE. Additionally, a study with bilateral intra-hippocampal administration at dose levels of 1×10⁹, 1×10¹⁰, and 1×10¹¹ vg/brain was performed. Intra-hippocampal injection of Construct B expressing both miR3bR and miR38R (double construct) into pilocarpine mice reduced hyperlocomotion, while administration of the AAV expressing only miR38R (Construct G) or only miR3bR (Construct H) did not reduce hyperlocomotion in the open field test at the doses tested (FIG. 3). Briefly, mice injected with 1×10¹⁰ copies of Construct B per brain showed normalization of the locomotor activity after injection, consistent with previous studies. Mice injected with 1×10⁸ or 1×10⁹ copies of Construct B per brain or with 1×10¹⁰ copies of Construct A per brain did not show a decrease of distance traveled after injection (FIG. 3).

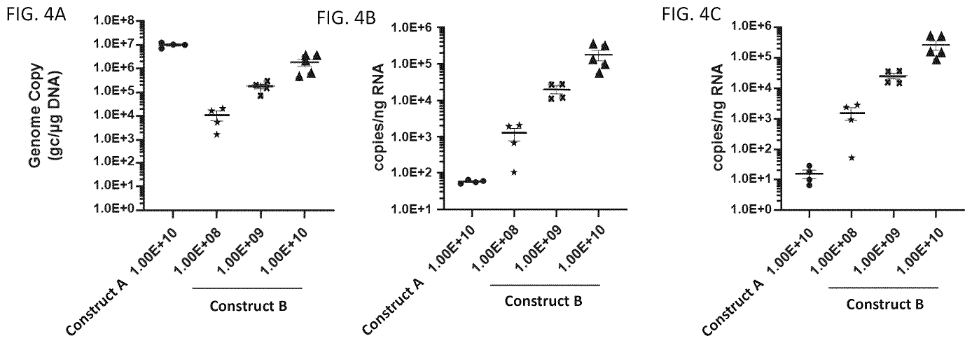

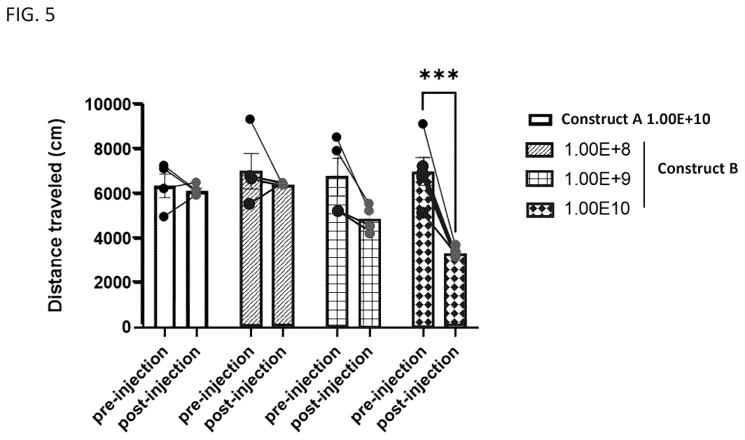

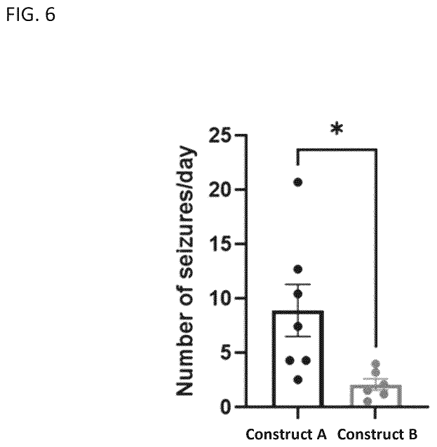

Pilocarpine-treated mice receiving a range of Construct B doses by bilateral intra-hippocampal administration showed dose-dependent increases in hippocampal expression of the vector genome, which correlates with increased expression of both miR38R and miR3bR (FIG. 4). Results showed that an averaged expression of 1×10⁵ copies/ng of RNA for both miR3bR and miR38R in hippocampi injected with Construct B at 1×10¹⁰ vg/brain was consistent with previous studies (FIG. 4). Also, the expression of miR3bR and miR38R followed a linear relationship between hippocampi injected with Construct B at 1×10⁸, 1×10⁹, and 1×10¹⁰ vg/brain. Results are presented as mean±SEM (Table 1). The hyperlocomotion of mice injected with Construct B at 1.0×10¹⁰ vg/brain was significantly reduced while the locomotion for mice injected with 1.0×10⁹ vg/brain showed improvement whilst 1.0×10⁸ vg/brain showed a lower level of improvement (FIG. 5). The dotted line represents the average level of activity of 20 naïve wild-type mice. Pilocarpine-treated mice injected with Construct B at 1×10¹⁰ vg/brain have a significantly lower number of seizures per day compared to pilocarpine mice injected with control (FIG. 6). In conclusion, we determined that the injection of constructs expressing miR3bR and miR38R in the hippocampus of pilocarpine-treated mice reduces hyperlocomotion, and the injection of constructs expressing only miR3bR or only miR38R in the hippocampus of pilocarpine-treated mice does not reduce hyperlocomotion.

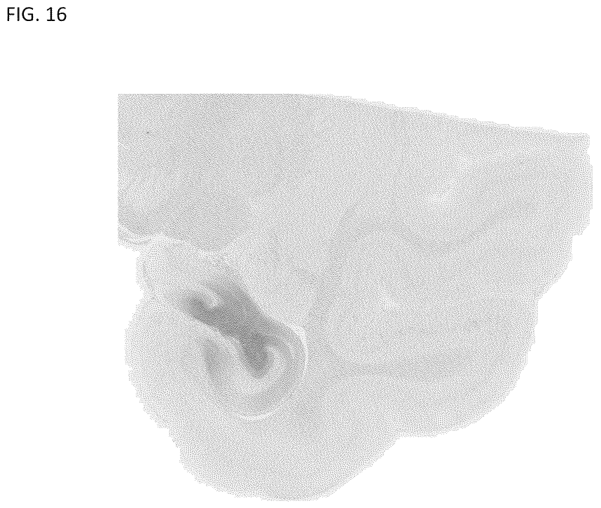

A study was conducted to investigate the biological activity of lower Construct B titer on EEG and locomotion readouts. Results from the study showed an averaged expression of 1×10⁶ vg/mg of DNA for Construct B injected at 1×10¹⁰ vg/brain and 1×10⁷ vg/mg of DNA for Construct A injected at 1×10¹⁰ vg/brain, consistent with previous studies (FIG. 16). Also, the expression of vector genome followed a linear relationship between hippocampi injected with Construct B at 1×10⁸, 1×10⁹, and 1×10¹⁰ vg/brain.

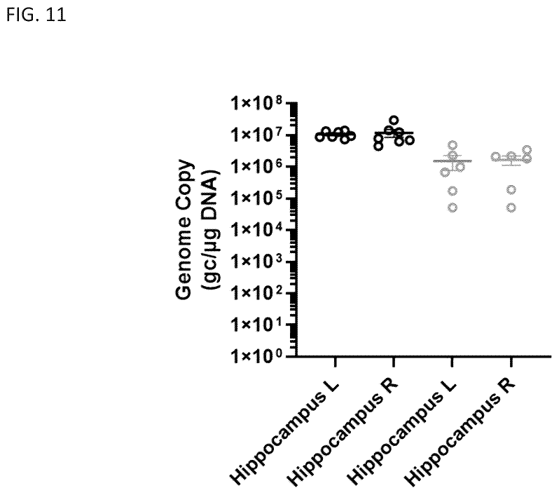

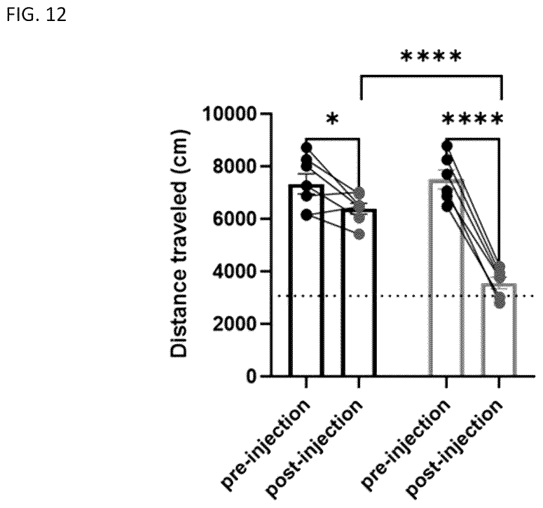

The results showed an average expression of 1×10⁶ vg/mg of DNA for Construct B, correlated with an averaged expression of 1×10⁵ copies of miR3bR/ng of total RNA and 1×10⁵ copies of miR38R/ng of total RNA (FIG. 11). Pilocarpine-treated mice injected with Construct B at 1×10¹⁰ vg/brain have decreased locomotion, comparable to levels observed in untreated naïve WT mice. Also, the distance traveled by mice injected with Construct B at 1×10¹⁰ vg/brain is significantly lower than that of pilocarpine-treated mice injected with Construct A (FIG. 12). Pilocarpine-treated mice injected with Construct A at 1×10¹⁰ vg/brain also showed a significant decrease in distance traveled (FIG. 12), probably due to the natural habituation to the open-field environment. This study confirmed that Construct B was effective in reducing the hyperlocomotion phenotype and also demonstrated that Construct B at 1×10¹⁰ vg/brain is effective in reducing the number of seizures per day.

In conclusion, injection of 1×10¹⁰ copies of Construct B in the hippocampus of pilocarpine-treated mice leads to an expression of miR3bR and miR38R, a decreased number of seizures, and a decrease in hyperlocomotion. Injection of Construct B with lower titers did not show a significant effect on the number of epileptiform activities measured with EEG or the hyperlocomotion of mice treated with pilocarpine. These results indicate that administration of 1×10¹⁰ vg/brain of Construct B effectively decreases seizures.

### Example 3: Three-Week Study Evaluating AAV9 Delivery Via Intra-Hippocampal Administration to Pilocarpine Mice

A total of 24 female, WT C57B16 mice were used for this study (n=21 for AAV9.CAG.GFP; n=3 for AAV9.hSyn.GFP). Mice received unilateral, stereotatically guided intra-parenchymal AAV injections into the hippocampus. Each animal received two 1 μL intra-hippocampal injections in the right hemisphere. Mice were necropsied 3 weeks post-dose.

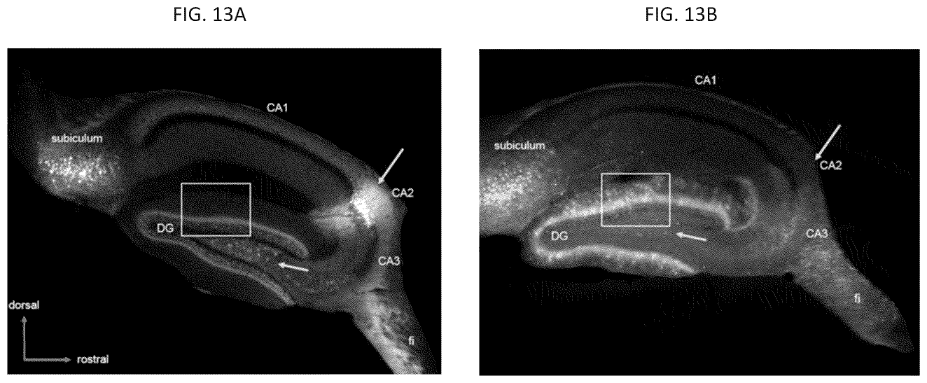

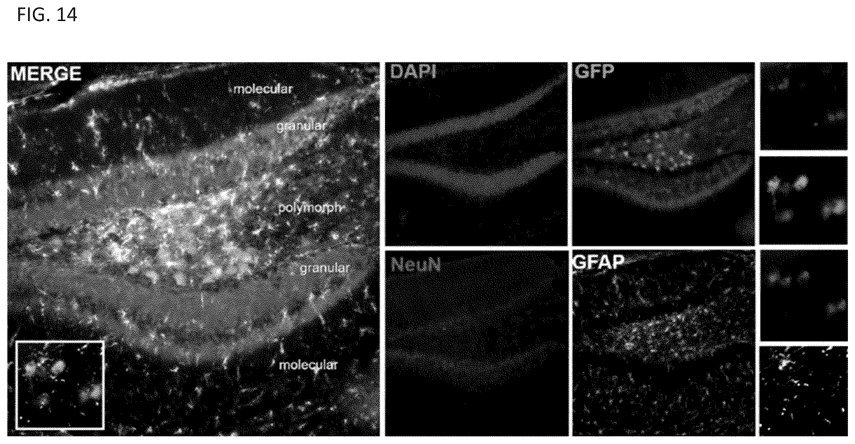

Important differences were observed when comparing CAG and human synapsin 1-driven GFP expression in the hippocampus. CAG expression is stronger than human synapsin 1 (FIG. 13), but importantly, the human synapsin 1 promoter restricts expression to neurons as demonstrated by co-localization with NeuN and lack of co-localization with GFAP compared to CAG, which targets both neurons and astrocytes (FIG. 14). GFP expression was restricted to neurons, indicating specificity of vector transduction.

In conclusion, in the pilocarpine-mouse model of epilepsy, Construct B, the expression of which is driven under the human synapsin 1 promoter, suppressed the translation or promotion of GRIK2 mRNA, leading to reduced GluK2 receptor expression, and was associated with improved seizure activity (EEG) and reduced hyperactivity. With this focused route of administration, expression of the vector was demonstrated to be largely restricted to the hippocampus and expressed predominantly in neurons. Little or no distribution of the AAV9 occurred in other regions of the brain or the peripheral organs.

### Example 4: Examination of Transduction Efficiency and Specificity of Vector in Cynomolgus Monkey

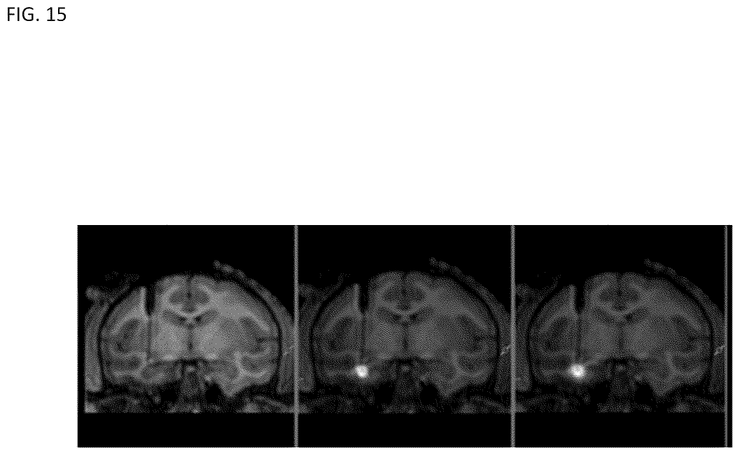

A study was performed using a preliminary vector, an AAV9-hSyn-GFP construct (Construct D), to demonstrate the feasibility of delivery of the construct to the hippocampus in the cynomolgus monkey using MRI-guided CED using a CLEARPOINT® Neuro System SMARTFLOW® cannula and to investigate sufficient transduction in the targeted area via GFP expression (FIG. 14, FIG. 15). This study provided guidance on the administration volume, and a preliminary assessment of biodistribution to the central nervous system (CNS), cerebrospinal fluid (CSF), blood, dorsal root ganglion (DRG), and selected organs such as liver and heart. This study demonstrated prominent expression of GFP (by immunohistochemistry) in the hippocampus with minor immunoreactivity (entorhinal cortex) and undetectable expression elsewhere in the brain and negligible distribution of the vector to the peripheral organs.

### Safety Evaluation and Biodistribution Studies in Cynomolgus Monkeys

Safety and biodistribution studies in cynomolgus monkeys are summarized in Table 5.

TABLE 5
Toxicity and Biodistribution Studies with Construct B

| | | | Test item/ | | |
|---|---|---|---|---|---|
| | Study | | Dose levels in | Sex and No. | |
| Study | Duration | | vg/hippocampus | per group | Endpoints |
| PK/PD safety study in wild | 42 | days | Diluent | 20 | histopathology |
| type Swiss mice following | | | Construct B: | males/group | AAV9 vector |
| injection of an rAAV | | | 1.0 × 10⁹, | | distribution |
| clinical candidate. | | | 1.0 × 10¹⁰, | | (qPCR) |
| | | | 1.0 × 10¹¹ | | Mature miRNAs |
| | | | vg/brain | | (SL RT-PCR for |
| | | | | | miR38R and |
| | | | | | miR3bR) |
| | | | | | GRIK2 mRNA |
| | | | | | (RT-PCR) |
| A 1-month dose range- | 4 | weeks | Construct B | 1/sex/group | Tolerability |

| | | | | |
|---|---|---|---|---|
| finding and biodistribution | | 60 μL, intra- | for vehicle | Histopathology |
| study of AAV 9-miRNA | | hippocampal, | control and | AAV9 vector |
| vector in cynomolgus | | bilateral, by CED | low dose | distribution |
| monkeys | | Dose levels in | 2/sex/group | (qPCR) |
| | | vg/hippocampus: | for mid- and | Mature miRNAs |
| | | Low dose: | high-dose | (SL RT-PCR for |
| | | 1.2 × 10¹⁰ | groups | miR3bR and |
| | | Mid dose: | | miR38R) |
| | | 1.2 × 10¹¹ | | GRIK2 mRNA |
| | | High dose: | | (RT-PCR) |
| | | 1.2 × 10¹² | | Immunogenicity |
| Toxicity study of intra- | Necropsies at | Construct B | 3/sex/group | Standard |
| hippocampal | 3 and 6 | 60 μL, intra- | for vehicle | toxicology |
| administration of AAV9- | months | hippocampal, | control group | endpoints |
| miRNA vector in | | bilateral, by CED | and low-, mid- | CNS, |
| cynomolgus monkeys | | Dose levels in | and high-dose | cardiovascular, |
| | | vg/hippocampus | groups for 6- | and respiratory |
| | | Low Dose | month | safety |
| | | 0.6 × 10¹¹ | necropsy | pharmacology |
| | | Mid Dose: | 1/sex for | Ophthalmology |
| | | 2.4 × 10¹¹ | vehicle | MRIs (during |
| | | High Dose: | control for 3- | infusion, at |
| | | 1.2 × 10¹² | month | 3 months and 6 |
| | | | necropsy | months) |
| | | | 2/sex/group | Clinical |
| | | | for mid- and | pathology |
| | | | high-dose | Histopathology |
| | | | groups for 3- | AAV9 vector |
| | | | month | distribution |
| | | | necropsy | (qPCR) |
| | | | | Mature miRNAs |
| | | | | (SL RT-PCR for |
| | | | | miR3bR and |
| | | | | miR38R) |
| | | | | GRIK2 mRNA |
| | | | | (RT-PCR) |
| | | | | Immunogenicity |

Efficacy of Construct B relative to control (diluent), administered via bilateral injection of 60 μl/hippocampus, was assessed by measuring the percent of GRIK2knockdown using qPCR (Table 6). MiRNA was quantified per ng DNA.

TABLE 6
GRIK2 Knockdown (KD)

| | vg/ | | | GRIK2 |
|---|---|---|---|---|
| Compound | hippocampus* | miR38R** | miR3bR** | KD |
| Construct B | 1.2 × 10¹² | 6.7 × 10⁵ | 1.1 × 10⁶ | −74% |
| Construct B | 1.2 × 10¹¹ | 1.5 × 10⁵ | 2.1 × 10⁵ | −19% |
| Construct B | 1.2 × 10¹⁰ | 2.7 × 10⁴ | 3.8 × 10⁴ | −16% |
| Diluent | NA | LLOQ | LLOQ | 0 |

### Example 5: Construct B Efficacy, Safety, and Biodistribution in Cynomolgus Monkeys

A dose-range study was performed using Construct B to assess safety and biodistribution of the vector, expression of mature miR38R and miR3bR, and GRIK2mRNA suppression at 4 weeks post dosing via MRI-guided CED using a CLEARPOINT® Neuro System SMARTFLOW® cannula. The high dose level in this study was used to test a possible maximum feasible dose, as well as a mid-dose and low-dose decrease in 10-fold increments.

The following assays were performed to support the studies in cynomolgus monkeys:
(1) Validated qPCR assay to support assessment of biodistribution of the AAV9 vector vector genomes;
(2) Qualified assays for bioanalysis of mature miRNA levels (stem-loop RT-PCR assays for miR3bR and miR38R) and GRIK2 mRNA levels (RT-qPCR assay) in tissues that are qPCR positive for AAV9; and
(3) Validated anti-AAV9 serum neutralizing antibody (Nab) assay.

### 1-Month Dose Range-Finding and Biodistribution Study of Construct B in Cynomolgus Monkeys

Dose levels were assessed for a subsequent toxicity and biodistribution study in cynomolgus monkeys. The study design is shown in Table 7. Twelve (6 male, 6 female) cynomolgus monkeys received bilateral infusions of diluent or Construct B into the hippocampus, delivered by CED using a CLEARPOINT® Neuro System SMARTFLOW® cannula. PROHANCE®, a gadolinium-based contrast agent (gadoteridol) was co-infused at a concentration of 2 mM to monitor the infusate distribution. Dose administration was guided by real-time MRI using the same trajectory and procedure as in the study with Construct D (GFP control). Animals received bilateral 60 μL infusions into the hippocampus; the volume remained the same for all injections. The dose was administered at a rate of 1 to 3 μL/minute according to the following study design. The high dose of 2×10¹³ vg/mL was considered a possible maximum feasible dose based on the volume administered and the vector titer of the stock (5.15×10¹³ vg/mL). The mid- and low-dose levels were selected to be 10-fold and a 100-fold lower vg/mL concentration than the high-dose level.

TABLE 7
Study Design of Dose-Range Finding and Biodistribution Study

| | | | | | Target | |
|---|---|---|---|---|---|---|
| | Number/ | | | Dose | Dose | Necropsy |
| | Sex of | Test | Concentration | Volume | (vg/ | Day |
| Group | Animals | Article | (vg/mL) | (μL)^{a} | hippocampus) | 29 ± 2 |
| 1 | 1M/1F | Diluent | 0 | 60 | 0 | 1M/1F |
| 2 | 1M/1F | Construct B | 2 × 10¹¹ | 60 | 1.2 × 10¹⁰ | 1M/1F |
| 3 | 2M/2F | Construct B | 2 × 10¹² | 60 | 1.2 × 10¹¹ | 2M/2F |
| 4 | 2M/2F | Construct B | 2 × 10¹³ | 60 | 1.2 × 10¹² | 2M/2F |

### Toxicity Study of Intra-Hippocampal Administration of AAV9-miRNA Vector in Cynomolgus

Monkeys

The design of the toxicity and biodistribution study is summarized below and in Tables 8 and 9.
Batch Construct B: 200 L toxicology batch manufactured with same process as GMP batch
Frequency of dosing: Single dose
Route and dose levels: Bilateral intra-hippocampal administration of 60 μL Construct B per hippocampus via CED using a CLEARPOINT® Neuro System SMARTFLOW® cannula and same procedure as in the dose-range finding study with Construct B and with Construct D (GFP control). The high dose was the same as in the dose-range finding study (2×10¹³ vg/mL; 1.2×10¹² vg/hippocampus), which tested a possible maximum feasible dose (Construct B stock is 4.485×10¹³ vg/mL). The mid- and low-dose are 5-fold and 20-fold lower than the high-dose level.
Age at dosing: Approximately 2.5 years
Duration: Necropsies at 3 and 6 months post-dose to enable assessment of time course for mature miRNA expression at 4 weeks and at 3 to 6 months (GLP study post-dose). The same assays, AAV9 vector (validated qPCR), mature miRNA expression (qualified stem-loop RT-PCR), and GRIK2 mRNA (RT-PCR) were used for these studies.

TABLE 8
Toxicity and Biodistribution Study Design

| | Number | | | Dose | Target Dose | Necropsy | Necropsy |
|---|---|---|---|---|---|---|---|
| | of | Test | Concentration | Volume | (vg/ | Day | Day |
| Group | Animals | Article | (vg/mL) | (μL)^{a} | hippocampus) | 92 ± 2 | 183 ± 2 |
| 1 | 3M/3F | Diluent | 0 | 60 | 0 | 1M/1F | 3M/3F |
| 2 | 3M/3F | Construct B | 1 × 10¹² | 60 | 0.6 × 10¹¹ | | 3M/3F |
| 3 | 5M/5F | Construct B | 4 × 10¹² | 60 | 2.4 × 10¹¹ | 2M/2F | 3M/3F |
| 4 | 5M/5F | Construct B | 2 × 10¹³ | 60 | 1.2 × 10¹² | 2M/2F | 3M/3F |

M = Male; F = Female

Animals received bilateral 60 μL infusions into the hippocampus.

TABLE 9
Parameters Assessed in the Toxicity and Biodistribution Study

| Parameters Assessed | Occasions (Week) | Details |
|---|---|---|
| Formulation Analyses | | Each day of dosing plus pre- and post-infusion |
| | | samples per animal |
| Bodyweights | Weekly | All animals |
| Clinical Observations | At least twice daily | All animals |
| | Pre-study, | Detailed clinical observations |
| | approximately 6 h post | Physical examinations |
| | dose, weekly, and | |
| | prior to necropsy | |
| | Pre-study, prior to | |
| | necropsy | |
| Functional observational | Pre-study and on Days | All animals |
| battery | 2, 8, 29, 92 ± 2, and | Mental status, gross motor movements, |
| | 183 ± 2 | proprioception, paresis, ataxia, dysmetria, |
| | | cranial nerve assessments, cervical spinal |
| | | assessments, and lumbosacral spinal |
| | | assessments. |
| | | Respiratory rate and body temperature also |
| | | assessed. |
| Ophthalmoscopy | Pre-study and on Days | All animals |
| | 29 ± 4, 92 ± 4, and | Standard observations using a binocular |
| | 183 ± 4. | indirect ophthalmoscope |
| Cardiovascular safety | Pre-study and Days | All animals |
| pharmacology | 92 ± 2 and 183 ± 2 | Electrocardiography examinations (Leads I, II, |
| | | and III) and blood pressure measurement |
| | | (systolic and diastolic) via tail cuff |
| MRI to assess CNS | Day 92 ± 2 and Day | All animals |
| changes | 183 ± 2. | Study Day 92 ± 2 and Study Day 183 ± 2 |
| Clinical pathology | Hematology, | All animals |
| | Coagulation, and | Parameters in accordance with common |
| | Clinical Chemistry: | practice |
| | Pre-study and Days | CSF: erythrocyte count, leukocyte count (total |
| | 29 ± 2, 92 ± 2, and | and differential), color and clarity, total protein, |
| | 183 ± 2 | glucose, albumin, chloride, |
| | Urinalysis: Pre-study | Phosphate, sodium, calcium, potassium |
| | and Days 92 ± 2 and | |
| | 183 ± 2 | |
| | CSF Analysis: Pre- | |
| | study and Days 92 ± 2 | |
| | and 183 ± 2 | |
| Immunogenicity | Pre-study, Months 3 | All animals |
| assessment: | and 6 | Collect samples for serum ADA analyses (total |
| | | binding ADAs and Nabs) |
| BIODISTRIBUTION | Study Days 2, 8, 15, | All Construct B-treated animals |
| AAV9 vector shedding | and 29 ± 2. | Urine, Nasal Swab, and Saliva |
| (qPCR) | Blood: 6 hours, | All Construct B-treated animals |
| AAV9 vector distribution | 24 hours, 72 hours, | All Construct B-treated animals |
| (qPCR) | 168 hours post-dose, | All Construct B-treated animals scheduled for |
| CSF Biodistribution | and 29 ± 2 days post | sacrifice |
| (qPCR) | dose | Days 29 ± 2, 92 ± 2, and 183 ± 2 |
| Biodistribution in tissues | CSF: Day 29, Month 3 | |
| | and Month | |
| | Month 3 and Month 6 | |
| | necropsies | |
| Mature miRNA expression | CSF: EOI, D 29 | All Construct B-treated animals |
| | Tissues: hippocampal | Stem-loop RT-PCR assays for miR38R and |
| | punches and selected | miR3bR |
| | CNS tissues | |
| GRIK2 mRNA levels | Hippocampal punches | All animals |
| | and selected CNS | RT-PCR assay |
| | tissues | |
| Weeks 13 and 26: | Termination | All animals scheduled for termination at Month |
| Necropsy, macroscopic | | 3 and Month 6 |
| examination, and organ | | |
| weights: | | |
| Histopathology | Detailed H and E | All animals scheduled for termination at Month |
| | neuropathology on | 3 and Month 6 |
| | hippocampus and full | All animals |
| | tissue list of peripheral | |
| | organs | |
| | IHC for glial cell | |
| | markers (iBa1 and | |
| | GFAP) | |

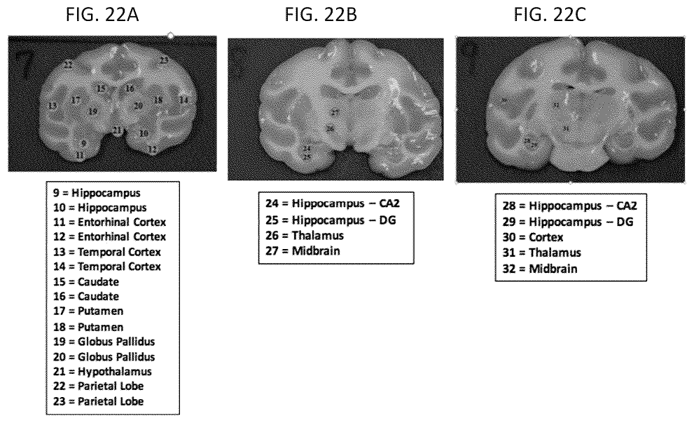

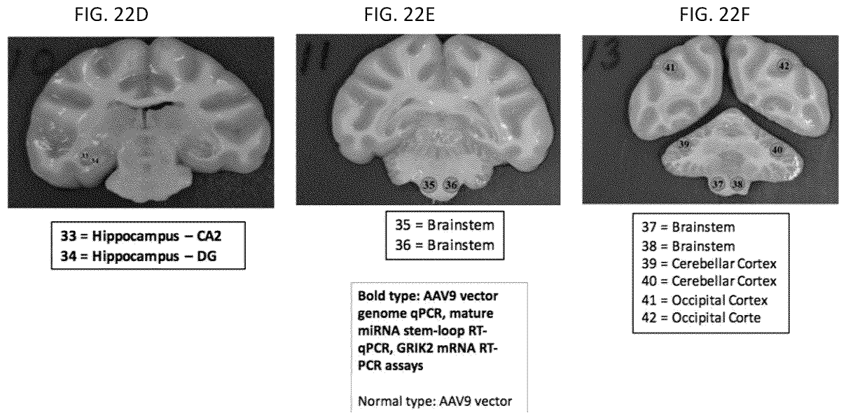

In-life observations and measurements included bodyweight, clinical observations, functional observation battery parameters (pre-study, on Study Days 2, 8, 15, 22, and prior to necropsy), and clinical pathology parameters (pre-study, Day 15, and prior to necropsy). Blood and CSF were collected for immunogenicity and biodistribution assessment. On Day 29+2, the animals were sacrificed, and selected tissues were harvested for histopathological and biodistribution evaluation. Brain biodistribution and mature miRNA expression/GRIK2 mRNA levels were assessed in 4 mm brain punches according to the brain punch map in FIG. 22.

The brain was sliced coronally at 3 to 4 mm slice thickness. 4 mm brain punches were collected according to the map, and analyzed for AAV9 vector genome, mature miRNA expression, and GRIK2 mRNA levels. The results of this study demonstrated the following:
Construct B was clinically well tolerated with no adverse events observed during the study.
There were no test article-related changes in bodyweights, functional observational battery parameters, or clinical pathology parameters (hematology, serum chemistry, coagulation) for male or female animals over the course of the study.
The dosing solution samples from Groups 2 through 4 were 62% to 125% of the adjusted nominal values, verifying the accuracy of formulation preparation from the stock test-article vector. The device compatibility samples from Groups 2 through 4 demonstrated negligible vector loss, if any, through the device and consistent delivery of vector concentration. Overall, the data confirmed the device-vector formulation compatibility.
Retrospective analysis of the MRI brain images were performed to estimate infusate distribution within and outside the hippocampus and to assess newly developed T2-weighted MR signal abnormality within the gadoteridol-infused target region on Day 15±2 and prior to necropsy on Day 29±2 using IPLAN® Flow. Newly developed T2 signal abnormality was not detected on the images obtained on Days 15 and 29 post-dose.
   Anatomic pathology data results demonstrated that:
There were no Construct B-related gross lesions for any of the animals. There were no Construct B-related changes in absolute or relative organ weights for either sex.
Bilateral intra-hippocampal administration of 60 μL of Construct B at a dose of 1.2×10¹⁰ vg/hippocampus (n=1/sex/group; hippocampus from only 1 animal available for examination; Group 2) was not associated with microscopic test-article effects in the examined sections.
Bilateral intra-hippocampal administration of 60 μL of Construct B at a dose of 1.2×10¹¹ vg/hippocampus of Construct B (n=2/sex/group; Group 3) was interpreted to be associated with slight glial reactions in the hippocampus and adjacent cerebral cortex as well as slight mononuclear cell infiltrates localized to the meningeal and perivascular space in the region of the hippocampus.
Bilateral intra-hippocampal administration of 60 μL of Construct B at a dose of 1.2×10¹² vg/hippocampus of Construct B (n=2/sex/group; Group 4) was associated with minimal-to-mild hippocampal neuron necrosis (4001M Grade 1: <1% neurons affected; 4501F Grade 2: 1-5% neurons affected), more prominent glial reactions and mononuclear cell infiltrates within the hippocampal gray matter and/or adjacent cerebral cortex, and mononuclear cell infiltrates within the meningeal and perivascular space in the region of the hippocampus and adjacent cerebral cortex.
   Table 10 provides additional details of the hippocampal microscopic findings. Gliosis was assessed by H&E; microgliosis was assessed by iBa1 immunostaining. Neuronal necrosis grades were assigned as follows: Grade 1, <1% neurons affected; Grade 2, 1 to 5 neurons affected. Hippocampal tissue from animal 2001M was inadvertently not processed.

TABLE 10
Severity Gradings for Microscopic Findings in Right Hippocampus in Control Group
(1001M, 1501F), Low-Dose (2001M, 2501F), Mid-Dose (3001M, 3002M,
3501F, 3502F), and High-Dose Monkeys (4001M, 4002M, 4501F, 4502F)

| | 1001 | 150 | 2001 | 250 | 3001 | 3002 | 350 | 350 | 4001 | 4002 | 450 | 450 |
|---|---|---|---|---|---|---|---|---|---|---|---|---|
| | M | 1F | M | 1F | M | M | 1F | 2F | M | M | 1F | 2F |
| Right | | | N/A | | | | | | | | | |
| Hippocampus | | | | | | | | | | | | |

GRAY MATTER

| | | | | | | | | | | | |
|---|---|---|---|---|---|---|---|---|---|---|---|
| Astrocytosis | — | — | — | — | — | — | — | — | — | — | 1 |
| Gliosis | 1 | 1 | — | — | — | 1 | — | 2 | 2 | 2 | 3 |
| Infiltrate, | — | — | — | — | — | — | — | — | — | — | 2 |
| mononuclear cells | | | | | | | | | | | |
| Microgliosis | | | — | 1 | 1 | 2 | 2 | 3 | 2 | 2 | 2 |
| Necrosis, | — | — | — | — | — | — | — | 1 | — | 2 | — |
| neuronal | | | | | | | | | | | |
| Infiltrate, | — | — | — | — | — | 2 | 1 | 2 | 2 | 2 | 3 |

mononuclear cells

WHITE MATTER

| | | | | | | | | | | | |
|---|---|---|---|---|---|---|---|---|---|---|---|
| Microgliosis | — | 2 | — | 1 | — | 1 | 1 | — | 2 | 2 | 2 |

The experimental procedures (including lumbar CSF tap) were associated with injection sites in multiple brain regions, variable glial reactions, slight inflammatory reactions (mononuclear cell infiltrates or inflammation), and slight nerve fiber degeneration in multiple brain regions, spinal cord, and/or spinal nerve roots. There did not appear to be an obvious test-article exacerbation of changes associated with the experimental procedures.

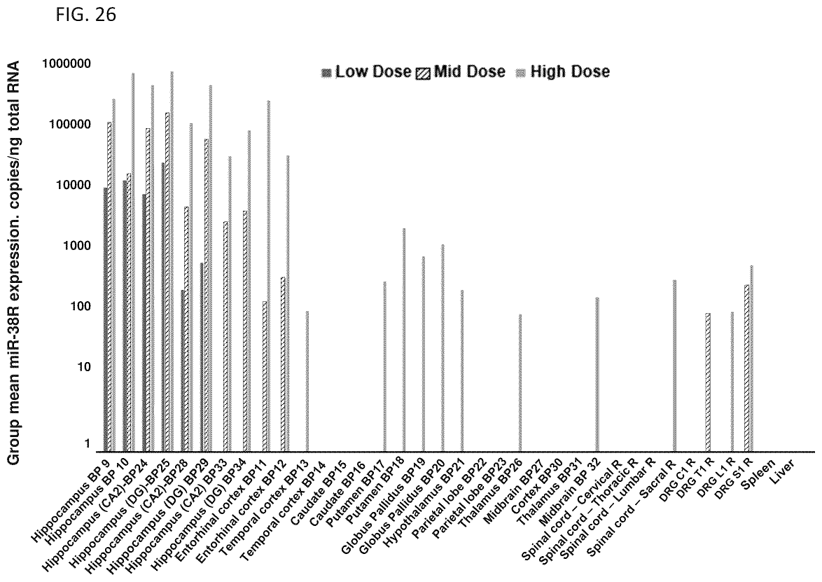

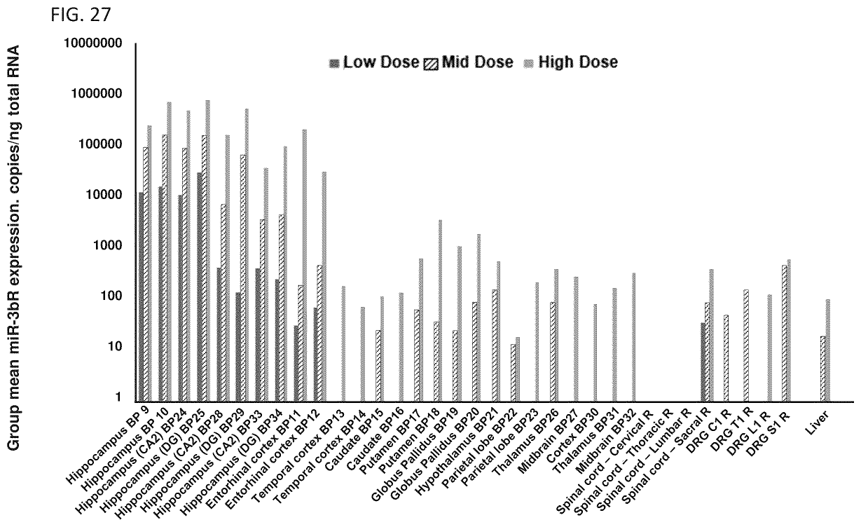

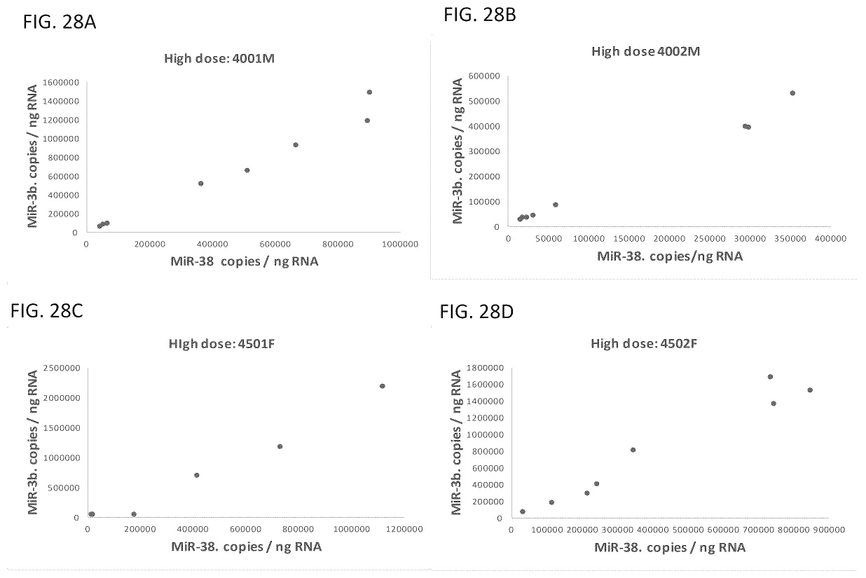

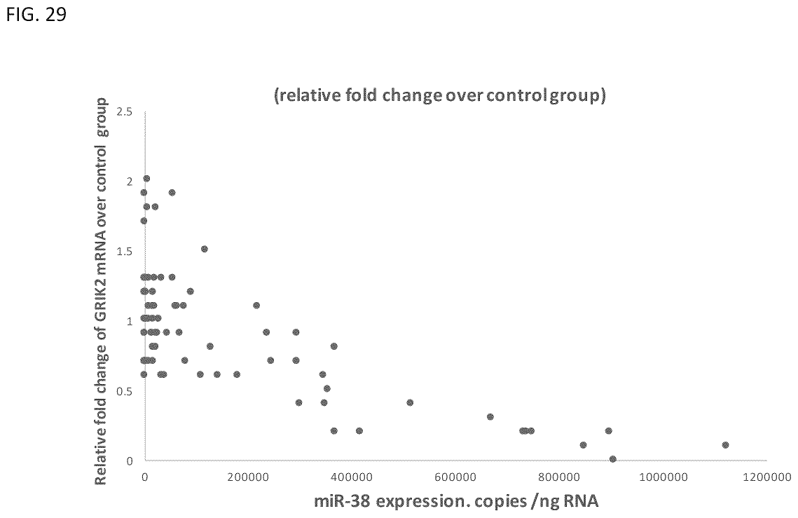

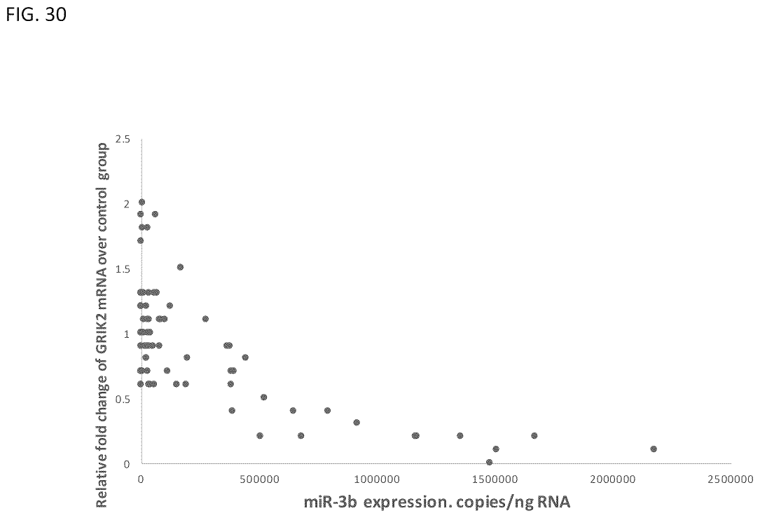

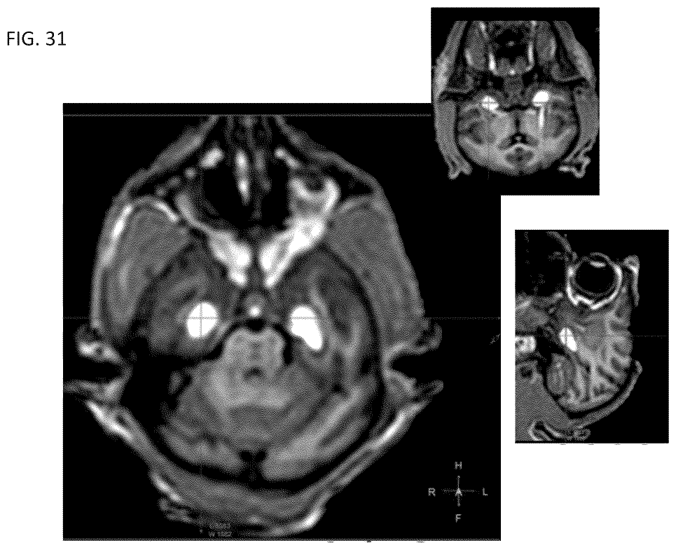

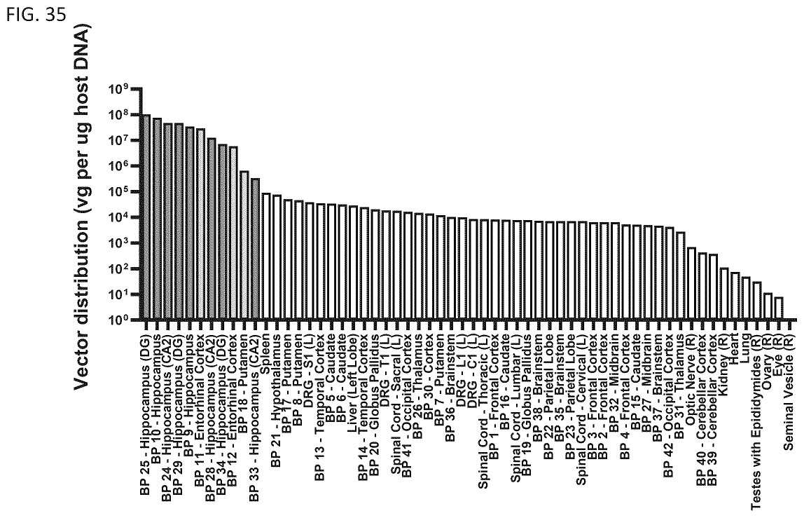

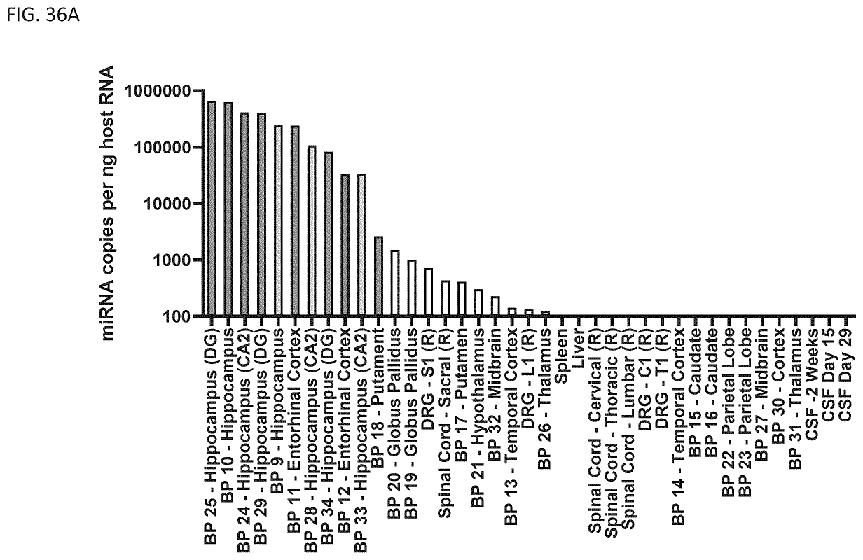

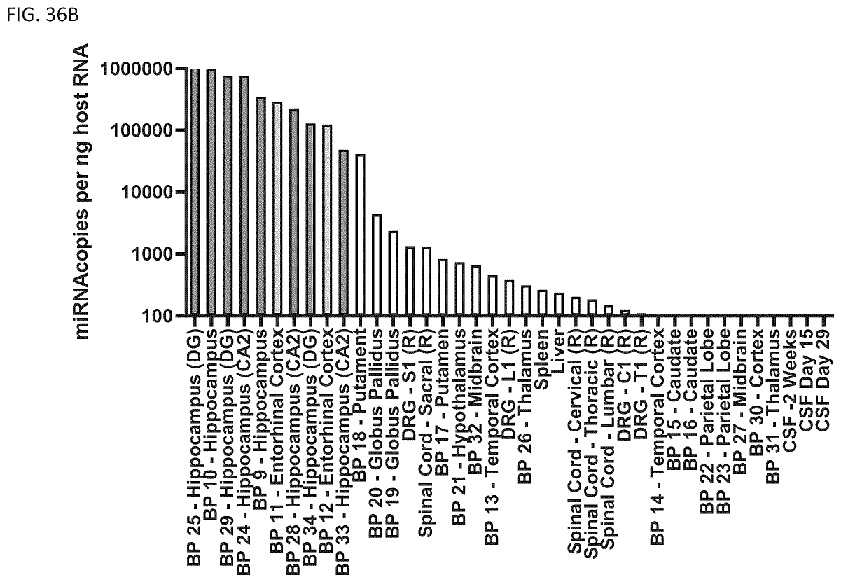

Test-article effects were not identified in the eyes with optic nerve, spinal cord, spinal nerve roots and ganglia, heart, kidney, liver, gallbladder, lung, spleen, ovary, testes, epididymis, or seminal vesicle. Biodistribution and molecular analyses results demonstrated the following:
AAV9 vector distribution, mature miRNA expression, and GRIK2 mRNA levels were assessed in each individual brain punch following a dual tissue extraction for DNA and RNA. QCd qPCR data for vector genome distribution are available:
High levels (1×10⁶ to 1×10⁸ vg/μg DNA) of vector genome were observed in the hippocampus in all Construct B-treated animals, and entorhinal cortex in a few high-dose animals (FIG. 23). These levels compare to the 1×10⁶ vg/μg DNA observed in pilocarpine mice at the efficacious dose level of 1×10¹⁰ total copies/brain).
Levels of vector genome in most other parts of the cynomolgus monkey brain were >100-1000-fold lower than in the hippocampus (FIG. 27).
Levels of vector genome in spinal cord, DRGs, optic nerve, spleen, and liver were about 1000-fold lower than the hippocampus (FIG. 26). CSF levels were very low by Day 29 post-dose (Error! Reference source not found.4).
Levels of vector genome vg in the lung, heart, kidney, testes/epididymides, seminal vesicles, ovary, and eye were negligible (FIG. 23).
There was a good correlation of vg and miRNA expression in hippocampal brain punches, as exemplified by miR3bR in Error! Reference source not found.
   QCd mature miRNA expression (stem-loop RT-qPCR assay) data for miR38R and miR3bR, and RT-qPCR data for GRIK2 mRNA demonstrate the following:
Dose-related expression of mature miR38R and miR3bR was observed in the hippocampal brain punches and levels were markedly higher than in other brain regions (Error! Reference source not found. and 31). Mature miRNA expression at levels of at least 1×10⁵ copies/ng total RNA, the levels associated with efficacy in mice (at 1×10¹⁰ vg/brain), were observed at the mid- (1.2×10¹¹ vg/hippocampus) and high-dose levels (1.2×10¹² vg/hippocampus) in monkeys (FIG. 27 and FIG. 28). Levels of mature miRNAs in the entorhinal cortex were also significant in the high-dose group but levels in other brain areas and spinal cord/dorsal route ganglia were 100-1000-fold lower or not detectable (FIG. 29 and FIG. 30). Levels of mature miRNA expression in liver and spleen were negligible.
There is a good correlation between miR3bR and miR38R mature miRNA expression in each hippocampal punch from individual animals as exemplified by the 4 high-dose animals (FIG. 31)
Levels of expression of mature miRNAs in hippocampal punches from the same brain slab varied between animals, likely dependent on the actual location of injection in the hippocampus in each animal. There was some evidence for slightly higher levels of miRNA expression in the DG versus CA2 region of the hippocampus (FIG. 35 and FIG. 36).

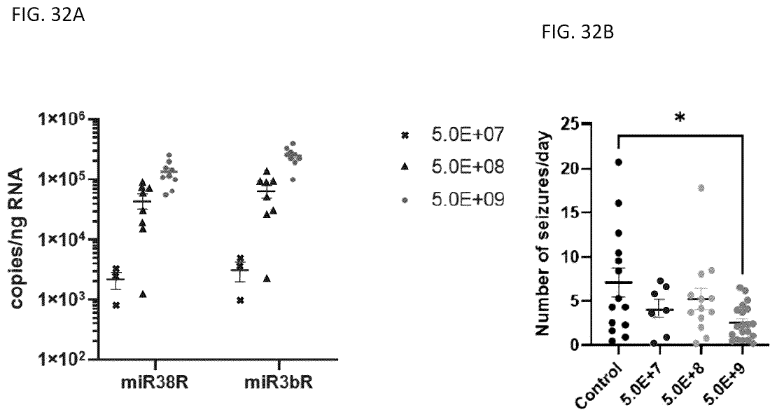

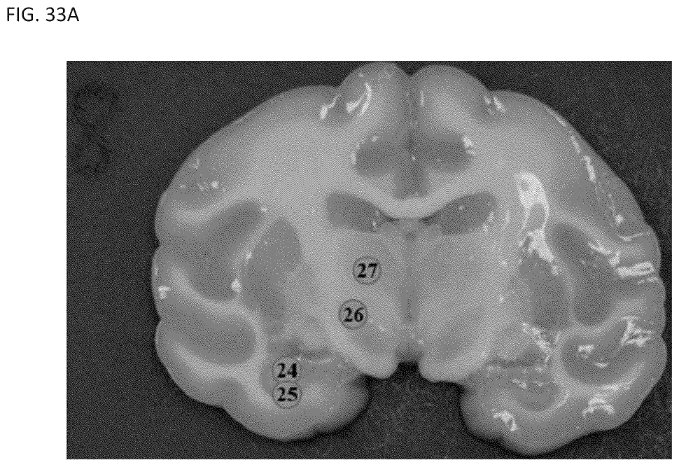

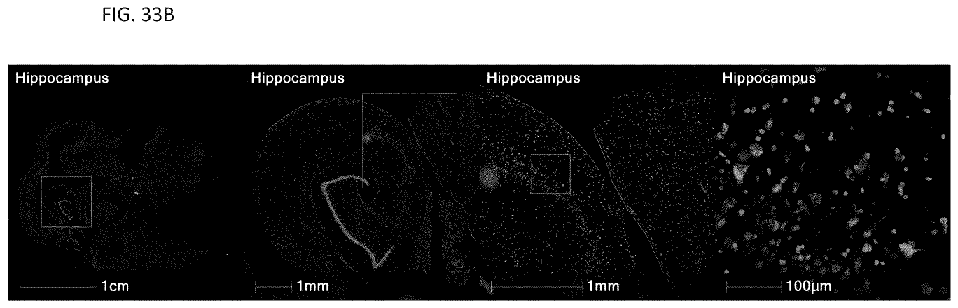

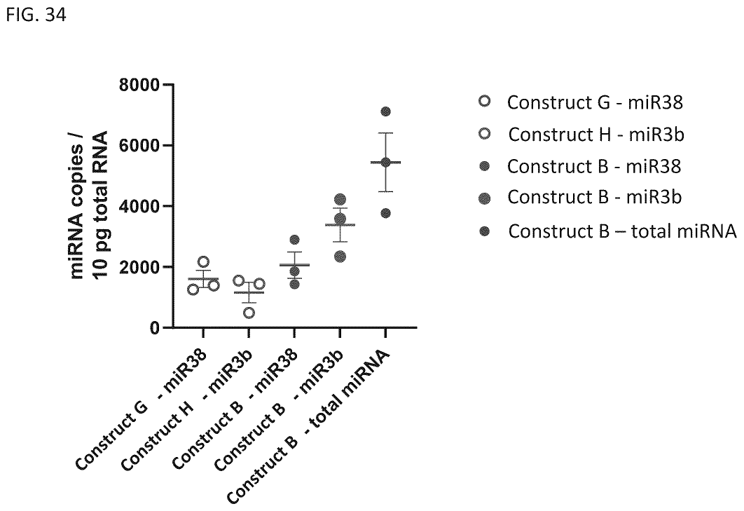

A correlation is apparent between expression of miR3bR and miR38R and GRIK2 mRNA levels in hippocampal brain punches (FIG. 32 and FIG. 33). A reduction as high as 90% of GRIK2 mRNA occurred in hippocampal tissues of Group 4 animals, such as brain punch 10 of animal 4501, punch 24 of animal 4502, and punch 25 of animal 4001. The highest group mean reduction of GRIK2 mRNA was associated with Group 4 hippocampus punch numbers 24 and 25 at 72% and 75%, respectively. There was a notable intra- and inter-animal variability in the values for relative fold change of GRIK2 mRNA over control group for brain punches in general. Fifty percent or greater reductions in GRIK2 mRNA, a level clearly above the variability in control brain punches, were associated with >200,000 copies/ng total RNA of each miRNA (FIG. 33 and FIG. 34). Based on these expression levels of mature miRNA, 3 mid-dose monkeys (3002M, 3501F, 3502F) and all 4 high-dose monkeys achieved, or were approaching, this miRNA expression level. The entorhinal cortex in 1 high-dose animal (4001M) also exceeded this miRNA expression level and showed decreased GRIK2 mRNA levels.

Five animals were negative for NAbs to AAV9 vector at the pre-screening timepoint; 1 remained negative at pre-dose, but all animals had titers<10 at pre-screening and pre-dose timepoints. NAb titers increased in all animals by Day 15 post-dose, followed by a general decrease by necropsy. There was no apparent impact of NAb status on AAV9 vector biodistribution or safety parameters (Error! Reference source not found.).

In conclusion, Construct B was well tolerated in cynomolgus monkeys based on all in-life parameters assessed. There was evidence for a dose-related expression of both mature miRNAs, at sufficient levels in several mid-dose and all 4 high-dose monkeys to achieve at least a 40 to 50% reduction in GRIK2 mRNA levels in the hippocampus (reductions greater than the assay variability in control brain punches). Gliosis/microgliosis and neuronal necrosis (Grade 1/2) were observed in the hippocampus in 2 high-dose animals at 4 weeks following intra-hippocampal administration of Construct B.

### Example 6: Assessment of Method of Administration of Construct B in Cynomolgus Monkeys

A study was performed using Construct B administered by MRI-guided CED using a CLEARPOINT® Neuro System SMARTFLOW® cannula, with necropsies at 3 months and 6 months post-dose (3/sex/group at 6-month timepoint). The high dose level in this study was selected a possible maximum feasible dose with the mid-dose and low-dose decreased in 5-fold and 4-fold increments, respectively. An experienced neuropathologist read the slides for the vector study with Construct D (GFP control), the dose-range finding study, and the toxicity/biodistribution study in cynomolgus monkeys with Construct B. Furthermore, the same assays for AAV9 vector genome biodistribution (validated qPCR assay), mature miRNA expression (qualified stem-loop RT-qPCR assays for miR3bR and miR38R), and GRIK2 mRNA (qualified RT-PCR assay) were used in the dose-range finding and toxicity studies in cynomolgus monkeys, collectively providing time-course information over 4 weeks in the dose-range study to 3 months and 6 months post-dose for these biodistribution and molecular endpoints.

### Three-Week Study Evaluating AAV9 Delivery Via Intra-Hippocampal Administration to Cynomolgus Monkeys

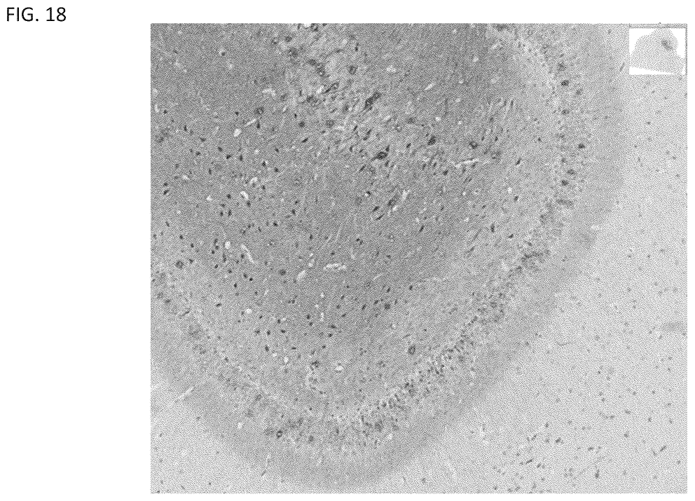

This study was performed with AAV9-Synapsin-GFP (Construct D) to demonstrate:
The top-down delivery (through top of skull—FIG. 18) of the AAV9-GFP to the hippocampus in the cynomolgus monkey
The selection of an administration volume
An assessment of biodistribution to the CNS, CSF, blood, DRG, and selected organs such as liver and heart

The volume administered in this study was 60 μL as a unilateral administration to 1 hippocampus (left). This volume is based on the results of a study with hippocampal injections of 1 to 2 μL of Construct D in healthy mice, and on scaling of hippocampal volume from published data in mice and monkeys. The animals were sacrificed 3 weeks post Construct B dosing and brain, spinal cord, and dorsal root ganglia (DRG) were examined microscopically and by enhanced GFP immunostaining (eGFP IHC). Brain, spinal cord, DRG, and peripheral organs, blood, and CSF were collected to assess biodistribution of AAV9 vector genome using a qPCR assay. This study design is presented in Table 11.

TABLE 11
Study Design

| | | | Dose | Target Dose | |
|---|---|---|---|---|---|
| | Number of | Concentration | Volume | (vg to Left | |
| Group | Animals | (vg/mL) | (μL) | Hippocampus) | Animal Numbers |
| 1 | 2 | 1 × 10¹³ | 60 | 0.6 × 10¹² | 1001, 1002 |
| 2 | 2 | 1 × 10¹² | 60 | 0.6 × 10¹¹ | 2001, 2002 |

The results of this study demonstrated that there were no test-item related effects on clinical signs or bodyweights following the intra-hippocampal administration of Construct D (Table 12), and MRI-guided intra-hippocampal delivery was successful based on the real-time MRI images as exemplified in FIG. 18. Dose volume is provided in μl per hippocampus, and target dose is provided in vg per hippocampus.

TABLE 12
Toxicology Study

| | | | Dose | Target | | |
|---|---|---|---|---|---|---|
| | Test | Concentration | Volume | Dose | Necropsy | Necropsy |
| Group | Article | (vg/mL) | (μL) | (vg) | 3 months | 6 months |
| 1 | Diluent | 0 | 60 | 0 | 1M/1F | 3M/3F |
| 2 | Construct B | 8 × 10¹¹ | 60 | 4.8 × 10¹⁰ | | 3M/3F |
| 3 | Construct B | 4 × 10¹² | 60 | 2.4 × 10¹¹ | 2M/2F | 3M/3F |
| 4 | Construct B | 2 × 10¹³ | 60 | 1.2 × 10¹² | 2M/2F | 3M/3F |

### GFP Immunostaining Results

Immunochemistry performed by labeling with eGFP demonstrated prominent immunoreactivity in the dosed (left) hippocampus of all animals with minor immunoreactivity in the region of the entorhinal cortex in 1 animal administered Construct D at a dose of 0.6×10¹¹ vg in the left hippocampus, and minor axonal immunoreactivity in the region of the thalamus in 1 animal administered Construct D at a dose of 0.6×10¹² vg (Table 13). No immunohistochemical labeling for eGFP was observed in the right (un-dosed) hippocampus, nor in other brain regions, spinal cord, or DRG.

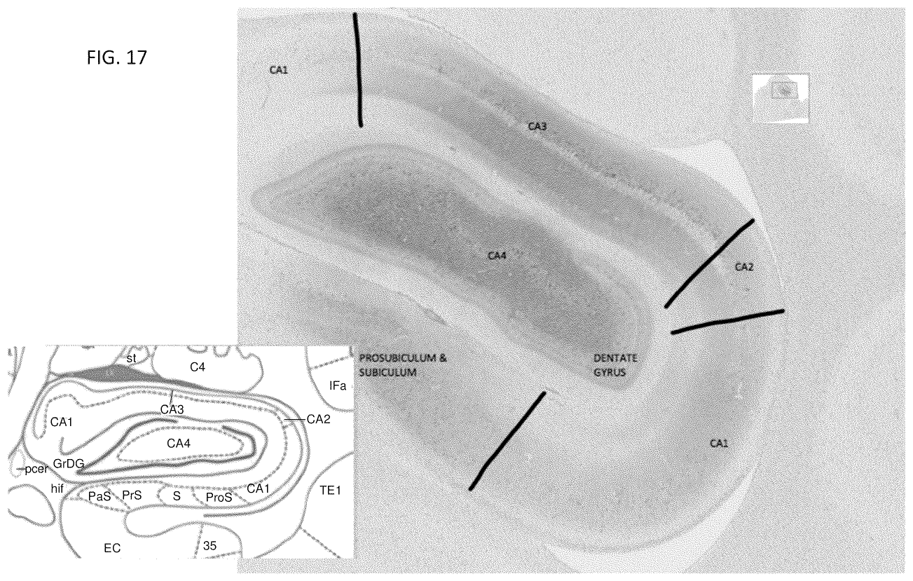

The intensity of eGFP labeling in the dosed hippocampus was generally higher in Group 1, where it was graded as severe (Grade 5), than in Group 2, where it was graded as moderate (Grade 3) to marked (Grade 4). This may indicate a dose-related effect, or it may be simply due to variation in the different levels of the hippocampus presented on the slides. Although there was some variation in labeling intensity across the different hippocampal subregions (dentate gyrus, CA1, CA2, CA3, CA4, and subiculum), these were not graded separately. There was no preferential tropism for a particular region of the hippocampus. Annotated images of the whole hippocampus for 1 animal are shown in FIG. 16, FIG. 17, and FIG. 18 (in increasing magnification of hippocampus for the same animal). Grading did not typically discriminate neuron bodies from axons as, in many cases, the entire parenchyma labeled strongly. However, hippocampal neuron cell bodies did often show prominent labeling.

TABLE 13
Summary of eGFP Immunoreactivity Grades

| | Group 1: 0.6 × 10¹² | | Group 2: 0.6 × 10¹¹ | |
|---|---|---|---|---|
| | vg/hippocampus | | vg/hippocampus | |
| | Animal | Animal | Animal | Animal |
| Tissue Diagnosis | 1001 | 1002 | 2001 | 2002 |
| Brain, Hippocampus, Left Hemisphere | Severe | Severe | Moderate | Marked |
| Percent-Positive eGFP Immunoreactivity | (Grade 5) | (Grade 5) | (Grade 3) | (Grade 4) |
| Brain Cerebral Cortex (Entorhinal), Left | — | — | Minimal | — |
| Hemisphere | | | (Grade 1) | |
| Percent-Positive eGFP Immunoreactivity | | | | |
| Brain, Thalamus, Left Hemisphere | Minimal | — | — | — |
| Percent-Positive eGFP Immunoreactivity | (Grade 1) | | | |

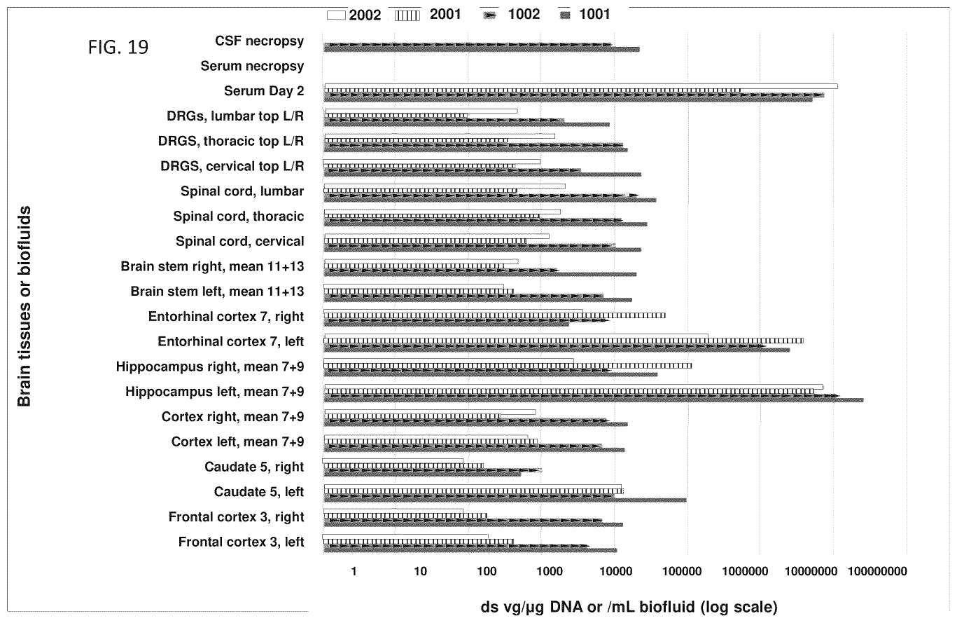

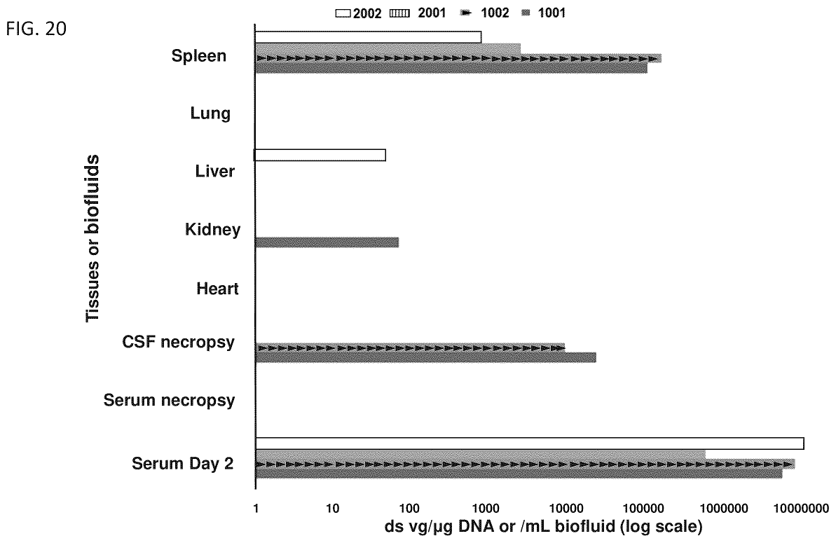

The results of assessing the biodistribution of the AAV9 vector genome demonstrated that the highest levels of AAV9 vector genome (qPCR assay) were detected in the left hippocampus. Levels in other parts of the brain, spinal cord, and DRG were 100-1000-fold lower than those in the left hippocampus, except for the entorhinal cortex. Levels in peripheral organs (liver, lung, heart, kidney) were negligible, but higher in spleen (FIGS. 19 and 20).

Studies of Construct B in NHPs confirmed a restricted distribution to the neurons of the hippocampus, with robust reductions of GRIK2 (90%) at the highest tested dose. In both mice and NHPs, Construct B was well tolerated with only minor findings at the site of delivery. A high dose of Construct B was associated with gliosis and minor neuronal necrosis in 2 NHPs after 4 weeks.

### Methods

### Cannula and Trajectory in the Cynomolgus Monkey Studies

Clinical studies using cynomolgus monkeys were performed using a CED system for administration of Construct B to the hippocampus. The following approach was employed:

### Cannula Delivery

The biodistribution and toxicology study was performed in the cynomolgus monkey using stereotactic MRI-guided administration via CED using a CLEARPOINT® Neuro System SMARTFLOW® cannula to administer Construct B. This cannula has been adapted for use in cynomolgus monkeys by a slight shortening of the cannula device, thus rendering it suitable for use in this species. The injection protocol is described below:

### Cannula Insertion and Construct B Delivery

The SMARTFRAMES® will be aligned to the target using iterative scanning and adjustments using the CLEARPOINT® system software. If the subject was randomized to Construct B treatment, the infusion process will begin as described below.

The dura will be punctured with a sharp lancet or a small dural opening will be made with a scalpel. The flow rate will be increased to 3 microliters/min for insertion, and a SMARTFLOW® cannula (NGS-NC-06) will be advanced to a position typically just at or within the border of the infusion target structure (hippocampus). After the initial insertion is complete, the flow rate will be turned down to 1 microliter/min. After cannula placement, the subject should be moved to isocenter of the MRI scanner and an MRI scan should be obtained to check the positioning of the cannula with the flow rate maintained at 1 microliter/min. Any errors in placement should be corrected at this point. Oblique coronal slab T1-weighted sequences (scan time approximately 2.5 to 3 minutes) along the trajectory of the cannula will be obtained on a continuous basis to monitor the infusions, with an axial slab T1-weighted scan through both putamen in the AC-PC plane performed approximately every 6 to 8 scans or according to local practice. Once infusate (i.e., gadoteridol) is seen at the tip of the cannula, the flow rate will be maintained until an infusion sphere has been established and at least reached the first step of the cannula (3 mm from the tip). At this point, the flow rate will be incrementally increased and the cannula progressively advanced through the infusion target (hippocampus) based upon the judgment of the neurosurgeon.

The target volume of the infusion is up to the maximal dose of 1200 microliters (±10 microliters to account for access to the pump between MRI scan sequences of several minutes each). However, the neurosurgeon may adjust the volume based upon individual anatomy to a maximum of 1600 microliters (±10 microliters). The flow rate may be increased from 1 to 3, 6, 9, 12, 15, 18, or 21 microliters/min, with a maximum of 18 microliters/min. The flow rates used and positioning/depth of the infusion during the infusion will be up to the judgment of the neurosurgical team based on MR visualization of the infusion to optimize fill of the target structure without overfilling based upon the neurosurgeon's judgment guided by intraoperative MRI. The cannula will generally be advanced or withdrawn in 2-3 mm increments. Maximal flow rates of 18 μl/min should not be reached until the primary step of the catheter at 13 mm is within the structure. The change in flow rate and depth will be guided by the evaluation of the infusions on MR imaging and the judgment of the neurosurgeon.

Trajectory into the Hippocampus

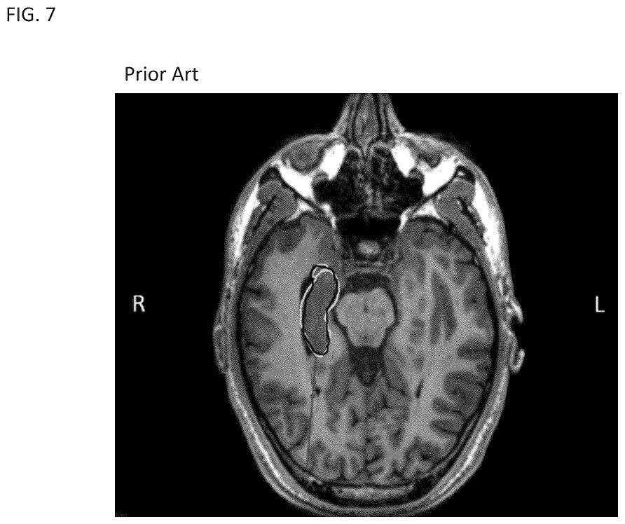

In humans, the administration trajectory for Construct B will be via the posterior route through the occipital cortex. The occipital route is well described in humans and is used to access the hippocampus for epilepsy treatments such as laser interstitial thermal therapy. The anatomy of the hippocampus is similar in humans and cynomolgus monkeys, and is located near the base of the brain, whereas in rodents there are significant anatomical differences and it is located much more dorsally. The trajectory to deliver the product into the monkey hippocampus (top-down orientation) will be different from that in humans (trajectory along the long axis of the amygdalohippocampal complex from an occipital entry point) (FIG. 7).

Access to the human hippocampus will be achieved through the occipital lobe as the occipital bone has little to no muscle attachment at the target entry site. In comparison, the occipital lobe in the cynomolgus monkey has a much flatter profile and is the site for significant muscle attachment. Occipital access in cynomolgus monkeys would require significant dissection of the neck musculature that attaches along the occipital crest. This dissection would add to the pain, distress, and time under anesthesia experienced by the test animals, markedly increasing the complexity and risk of the procedure. Conversely, selection of a “top-down” delivery route in the cynomolgus monkey represents an anatomically appropriate, readily available procedure that is frequently used by CROs experienced in parenchymal administration in this species. Surgical instrumentation has been developed specifically for “top-down” delivery in the cynomolgus monkey. The top-down approach has been used previously with cynomolgus monkeys and data with imaging, pathology, and analytical techniques demonstrate that the head and corpus of the hippocampus can be accurately targeted with a top-down approach.

### Bilateral Administration in NHPs

To ensure availability of sufficient hippocampal tissue for histology, immunohistochemical, qPCR vector biodistribution, mature miRNA and GRIK2 mRNA analysis and investigation of other exploratory endpoints, bilateral injections into the hippocampus were performed. A tabulated synopsis of the toxicity and biodistribution study is included. Effects of unilateral administration have been explored with AAV9-hSyn-GFP (Construct D). Results from this study show that unilateral intra-hippocampal delivery of Construct D leads to prominent GFP labeling in the treated hippocampus using eGFP immunohistochemistry, and no immunohistochemical labeling for eGFP in the contralateral (un-dosed) hippocampus, nor in other brain regions, spinal cord, or DRG.

### Pharmacokinetics

The bioanalytical strategy included the following assays to support the studies in cynomolgus monkeys:

### Methods of Analysis

qPCR Assay for AAV9 Vector Genome

A qPCR assay using primers to the polyA region of AAV9 has been validated for the quantification of Construct B vector biodistribution in cynomolgus monkey tissues. The lower limit of quantitation (LLOQ) at 25 copies per μg of gDNA for Construct B biodistribution in dosed cynomolgus monkey tissues is within the FDA recommendations of an assay with a demonstrated LOQ of 550 copies of vector per μg of host DNA. All reported runs met acceptance criteria and demonstrated assay sensitivity, specificity, intra- and inter-assay precision and accuracy, and reproducibility. Different laboratory operators or QS7 instruments did not affect the precision or accuracy of the analysis, demonstrating the overall ruggedness and robustness of the assay.

Stem-Loop RT-PCR Assays for Mature miRNAs

For brain punches, DNA and RNA samples for qPCR analysis of vector copy numbers and RT-qPCR analysis of miR38R, miR3bR, and GRIK2 mRNA analysis came from the same tissue lysates to maintain data consistency. The total RNA samples were analyzed for both vector-derived miRNA expression and miRNA-induced GRIK2 mRNA reduction. There are 3 steps involved in the analysis of gene expression samples:
RNA extraction
Reverse transcriptions of RNA into cDNA
qPCR analysis of the cDNA samples

A method for 2 stem-loop RT-qPCR assays has been qualified for quantification of vector-derived miR38R and miR3bR expression in brain tissue from cynomolgus monkeys. The 2 assays share the same reverse transcription. All reported runs met the acceptance criteria and demonstrated the assay sensitivity, specificity, intra- and inter-assay precision and accuracy, and reproducibility. The miR38R assay limit of detection (LOD), LLOQ, and upper limit of quantitation (ULOQ) are 500, 500, and 10⁸ copies of miR38R per qPCR or 5000, 5000, and 10⁹ copies of miR38R per RT, respectively. The miR3bR assay LOD, LLOQ, and ULOQ are 50, 50, and 10⁸ copies of miR-3bR per qPCR or 500, 1000, and 10⁹ copies of miR-3bR per RT, respectively. Different laboratory operators or QS7 instruments did not affect the precision or accuracy of the analysis, demonstrating the overall robustness of the assay. There was no detected matrix effect to analyze the spleen or CSF RNA samples using the qualified assays.

RT-PCR Assay for GRIK2 mRNA

Two 1-step single plex RT-qPCR methods (DDCt method) have been established and qualified for relative quantitation of cynomolgus monkey endogenous GRIK2 mRNA using the host reference HPRT1 mRNA. The GRIK2 primer/probe set specifically amplifies an 87-nt sequence of monkey GRIK2 cDNA, which overlaps with the miR38R-targeting sequence. The specificity of the assays' amplicons for the target mRNAs (GRIK2 and HPRT1) were demonstrated, and the accuracy and precision of the method are suitable for relative quantitation of GRIK2 mRNA in monkey tissues. Up to 3 freeze-thaw cycles did not have a significant effect on RNA stability. Different analysts or QS7 Flex instruments did not affect the precision or accuracy of the analysis, showing the overall ruggedness and robustness of the assay. The data show sensitivity, specificity, and linear range of the qPCR assay. The qualified LOD, LLOQ, and ULOQ per reaction for brain tissue are 0.39 ng, 1.56 ng, 400 ng for GRIK2 and 0.39 ng, 0.39 ng, and 400 ng for HPRT1.

### GLUK2 Receptor Protein

GluK2 protein expression in primary mouse cortical neurons cultures can be assessed by western blot. Both MS-based proteomic approaches and ELISA-based methods such as MSD and TR-FRET technologies could also be used.

### Immunogenicity Assays

A validated commercially available cell-based Nab assay for AAV9 in human serum (BioAgilytix US) has been cross-validated for use in cynomolgus monkey serum. A total binding serum ADA is being qualified for use in cynomolgus monkey serum.

We did not develop ADA assays for the mature miRNAs as these contain no non-natural/modified nucleotides and will generally be expressed intracellularly, or within exosomes in blood/CSF, and it is considered there will be a low risk of development of a humoral response to the expressed mature miRNAs. We also did not develop a PBMC-based IFNg ELISpot assay to assess cellular immune responses to AAV9 due to the local hippocampal delivery of the vector, high levels of transduction in the hippocampus, and generally minimal-to-mild histopathological findings noted so far in the brain. Thus, an assay to assess cellular immune responses to AAV9 is not considered to provide additional information for the interpretation of the toxicity and biodistribution study.

### Biodistribution of AAV9 and Construct B

Biodistribution studies with Construct D (AAV9.hSyn.GFP) following intra-hippocampal administration to mice and cynomolgus monkeys are summarized in Table 14. GFP expression in the hippocampus was shown to be restricted to neurons in mice; in monkeys GFP immunostaining was prominent in the hippocampus (neuron cell bodies showed prominent staining) following unilateral administration of Construct D, but generally negligible in other brain regions (including the un-dosed contralateral hippocampus) except for the entorhinal cortex of 1 of the 4 treated animals. Highest levels of AAV9 vector genome (qPCR assay) were detected in the dosed (left) hippocampus of the cynomolgus monkey given Construct D. Levels in other parts of the brain, spinal cord, and dorsal route ganglia were 100- to 1000-fold lower than those in the left hippocampus, except for the entorhinal cortex. Levels in peripheral organs (liver, lung, heart, kidney) were negligible, but higher in the spleen.

Biodistribution data for Construct B AAV9 vector, the expression of miR3bR and miR38R, the 2 expressed mature miRNAs, and GRIK2 mRNA levels have been evaluated in cynomolgus monkeys. Following administration of Construct B, high levels of mature miRNAs were present in the hippocampus, particularly in the areas closest to the site of administration. Levels of mature miRNAs were negligible in liver and spleen and about 1000-fold lower than hippocampal levels in spinal cord and dorsal root ganglia.

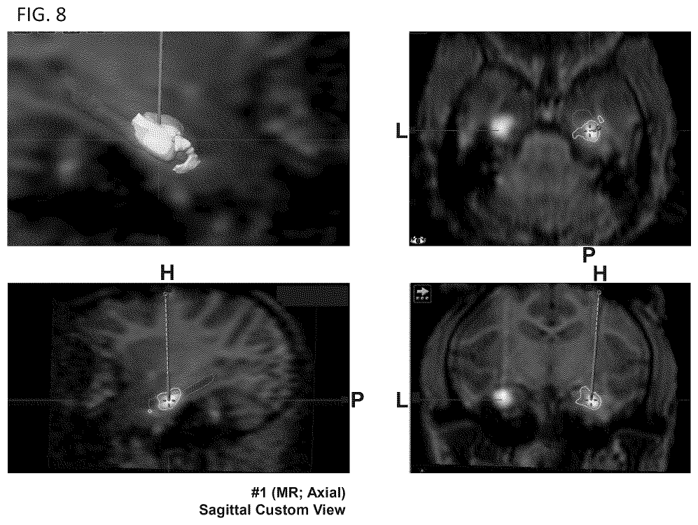

TABLE 14
Biodistribution Studies with Construct D (AAV9-Synapsin-GFP)

| | Study | Test item/ | Sex and | |
|---|---|---|---|---|
| | Duration | Dose levels | No. per | |
| Study | (Days) | vg/hippocampus | Group | Endpoints |
| AAV9 delivery via intra- | 3 weeks | Construct D | 3 females | GFP distribution |
| hippocampal administration | | (AAV9.hsyn.GFP) | | |
| to mice | | Two 1 μL intra- | | |
| | | hippocampal | | |
| | | injections of 1 × 10¹⁰ | | |
| | | vg to right | | |
| | | hemisphere | | |
| One month study | 3 weeks | Construct D | Males | AAV9 vector |
| evaluating AAV9 delivery | | (AAV9.hsyn.GFP) | 2/group | distribution (qPCR) |
| via intra-hippocampal | | 60 μL, intra- | | GFP distribution |
| administration to | | hippocampal, | | (IHC |
| cynomolgus monkeys | | unilateral, by | | immunostaining) |
| Top-down delivery route as | | convection | | Tolerability |
| shown in image of MRI- | | enhanced delivery | | Histopathology |
| guided delivery in FIG. 8. | | Group 1: 1.0 × 10¹³ | | |
| | | vg/mL (0.6 × 10¹² | | |
| | | vg/hippocampus) | | |
| | | Group 2: 1.0 × 10¹² | | |
| | | vg/mL (0.6 × 10¹¹ | | |
| | | vg/hippocampus) | | |

### Example 7: Construct B Efficacy Using Human Brain-Derived Organotypic Slices

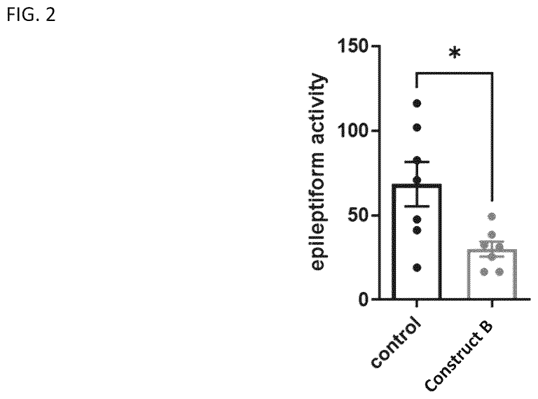

To determine the efficacy of Construct B in human brain tissue, organotypic hippocampal slices obtained from resection surgery conducted on patients with TILE were treated with Construct B and Construct D (GFP control), Construct A (null control), or Construct M as controls (Table 2 and FIG. 2). The epileptiform activity was induced by treatment with 5 μM gabazine and 50 μM 4-Aminopyridine, which constitutes the baseline for the experiment. The treatment with the different AAV constructs was applied after washout of gabazine and 4-Aminopyridine.

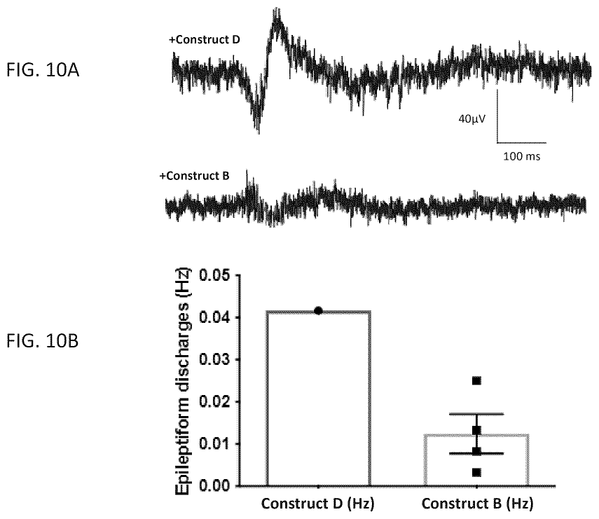

Results obtained from 7 donors showed that treatment with Construct B leads to a significant decrease in epileptiform discharges when compared to controls (FIG. 2). Patient-to-patient variability can be high. Results from organotypic slices obtained from 1 patient and treated with either Construct A (null control) or Construct B showed a decrease in epileptiform activity (FIG. 10).

In conclusion, Construct B treatment of organotypic slices from patients with TILE reduces epileptiform activity.

### Example 8: Off-Target Potential of Construct B Using in Silico and In Vitro Methods in Human Cells

The approach used for assessment of off-target effects followed the methods summarized by Keskin et al. (Molecular Therapy Methods & Clinical Development. 15: 275-284. 2019). Both on- and off-target effects were assessed in human induced pluripotent stem cells (iPSC) GlutaNeurons. This included an assessment of the potential for off-target effects of both guide and passenger strands and the potential for changes in endogenous miRNA expression and saturation of the endogenous RNAi machinery using small RNA sequencing.

Construct B induces a GRIK2 mRNA lowering effect based on a 21-22 nucleotides homology and may also bind and lower the expression of genes other than the GRIK2 gene. Since Construct B uses the human synapsin 1 promoter, which has been shown to restrict transgene expression to neurons, the off-target analyses will be restricted to neuronal cultures. An AAV9-hSyn1-GFP vector will be used to quantify the numbers of cells transduced (i.e., expressing GFP). The expression levels of miR3bR and miR38R and GRIK2 mRNA will be quantified to confirm sufficient expression levels and effective knock-down respectively.

Off-targets predicted by in silico methods were evaluated by RNAseq in samples of Construct B-treated iPSC GlutaNeurons compared with AAV9-hSyn1-GFP control vector and IT diluent at the same transduction multiplicity of infection (3.0×10⁵, 1.0×10⁶, and 3.0×10⁶ vg/cell). The use of AAV9-hSyn1-GFP allowed for quantification of the amount of cells expressing GFP and therefore transduced. The expression levels of miR3bR and miR38R and GRIK2 mRNA were quantified to confirm sufficient expression levels and effective knock-down, respectively. Genes with a significant up or downregulation between Construct B and control vector or IT diluent were further evaluated using real-time polymerase chain reaction (RT-qPCR) for confirmation and cellular pathways related to these genes will be investigated. For genes that were confirmed to be significantly differentially expressed, a safety risk assessment was provided.

Off-target activity was evaluated by next-generation full transcriptome sequencing investigating 2 aspects:
Small RNA analysis to evaluate possible changes of the cellular miRNA processing and unbiased transcriptome sequence analysis. Construct B was compared to Construct D (AAV9-hSyn1-GFP) and IT diluent. No significant increase or decrease in small RNA expression was observed.
Genes with a significant up or downregulation between Construct B and control vector or IT diluent was further evaluated using RT-qPCR for confirmation and cellular pathways related to these genes were investigated. Construct B activated the NGF-stimulated transcription pathway. Examples of genes of the NGF-stimulated transcription pathway regulated by Construct B include VGF (not an acronym—ENSG00000128564), NAB2 (NGFI-A Binding Protein 2-ENSG00000166886), CDK5R2 (Cyclin Dependent Kinase 5 Regulatory Subunit 2, ENSG00000171450), JUNB (JunB Proto-Oncogene, AP-1 Transcription Factor Subunit, ENSG00000171223) and JUND (JunD Proto-Oncogene, AP-1 Transcription Factor Subunit, ENSG00000130522).
A risk assessment was performed to assess any identified off-target genes that are dysregulated, followed by further in vitro studies. Treatment with Construct B did not increase or decrease the expression of genes involved in cellular pathways of concern, indicating that administration of Construct B does not produce significant off-target effects and is therefore safe for use as an anti-epileptic therapy.

### Intermediate Summary: Proposed Dosage Determination

Based on the mouse, cynomolgus monkey, and ex vivo, in vitro, and in silico human studies, summarized in Table 15 below, proposed dosages suitable for use in human subjects diagnosed with epilepsy (e.g., TILE or focal epilepsy) or displaying one or more symptoms of epilepsy (e.g., seizures) were determined.

TABLE 15
Seizure Reduction in GRIK2 KD

| | Total vg/ | vg/mm³ | miRNA/ng total RNA^{c} | | Seizure |
|---|---|---|---|---|---|
| Species | hippocampus | hippocampus² | (miR38R/miR3bR) | GRIK2 KD | reduction |
| Pilocarpine | 5.0 × 10⁹ | 2.5 × 1008 | 1.0 × 10⁵/1.9 × 10⁵ | TBD | −70% |
| Mouse | 5.0 × 10⁸ | 2.5 × 1007 | 3.0 × 10⁴/4.3 × 10⁴ | TBD | −37% |
| NHP DRF | 1.2 × 10¹² | 6.0 × 1009 | 6.7 × 10⁵/1.1 × 10⁶ | −74% | NA |
| | 1.2 × 10¹¹ | 6.0 × 1008 | 1.5 × 10⁵/2.1 × 10⁵ | −19% | NA |
| | 1.2 × 10¹⁰ | 6.0 × 1007 | 2.7 × 10⁴/3.8 × 10⁴ | −16% | NA |
| NHP GLP | 1.2 × 10¹² | 6.0 × 1009 | pending | pending | NA |
| | 2.4 × 10¹¹ | 1.2 × 1009 | pending | pending | NA |
| | 6.0 × 10¹⁰ | 3.0 × 1008 | pending | pending | NA |
| Human | 3.6 × 10¹² | 1.2 × 1009 | | | Clinical |
| Proposed | | | | | Endpoint |
| Dose | 9.0 × 10¹¹ | 3.0 × 1008 | | | Clinical |
| | | | | | Endpoint |

### Example 9: Treatment of an Epilepsy in a Human Subject by Administration of a Viral Vector Encoding One or More Inhibitory Poly Ribonucleotides Targeting a GRIK2 mRNA

A subject, such as a human subject (e.g., a pediatric or adult subject) diagnosed as having an epilepsy (e.g., a TILE, such as, e.g., mTLE or ITLE), can be treated with a composition (e.g., an AAV vector encoding a ribopolynucleotide that inhibits GRIK2; e.g., Construct B) described herein to reduce one of more epilepsy symptoms including, but not limited to one or more of (e.g., 2 or more, 3 or more, 4 or more of): (a) risk of seizure recurrence; (b) reduction of excitotoxicity and associated neuronal cell death in the CNS; (c) restoration of a physiological excitation-inhibition balance in the affected region of the CNS; (d) reduction in one or more symptoms of a epilepsy (e.g., frequency, duration, or intensity of epileptic seizures, weakness, absence, sudden confusion, trouble understanding or producing speech, cognitive impairment, impaired mobility, dizziness, or loss of balance or coordination, paralysis, and emotional dysregulation), and (e) pathological sprouting of recurrent mossy fibers of dentate gyrus granule cells in the hippocampus. The method of treatment can optionally include diagnosing or identifying the subject as a candidate for treatment with a composition of the disclosure before administration. The subject can be, for example, less than 65 years of age and is diagnosed with or exhibits one or more symptoms of epilepsy, in which, for example, the subject: (a) experienced at least 12 documented seizures during the previous 90 days, such that, e.g., the seizures include (i) at least 2 documented focal impaired awareness seizures, and/or (ii) at least 10 documented focal aware seizures, (b) experienced no 21-day seizure-free period in the previous 90 days, (c) has confirmed hippocampal atrophy, as determined by, e.g., MRI-T1 imaging, optionally with (i) increased ipsilateral mesial signal on T2 imaging or (ii) ipsilateral hypometabolism on fluorodeoxyglucose positron emission tomography (FDG-PET), (d) scores 23 or above on a Montreal Cognitive Assessment (MoCA), and/or (e) has no significant focal neurocognitive dysfunction, inconsistent with disease pathology-related magnetic resonance imaging (MRI) and positron emission tomography (PET) imaging findings. In addition, the subject may be one that does not have: (f) lesions on neuroimaging outside of the mesial temporal love area, temporal neocortical or extratemporal lesions on MRI, or diffuse unilateral or bilateral hypometabolism on PET, (g) any progressive neurological disorder, (h) psychogenic seizures within the last 2 years, (i) implanted devices that would contraindicate MRI-guided convection-enhanced delivery (CED), such as vagus nerve stimulation [VNS] devices and cochlear implants, (j) previous major disease-unrelated neurosurgical intervention due to intracranial tumor, trauma, or bleeding, (k) medical history of schizophrenia, (I) medical history or current assessment of suicidal ideation or suicide attempt, as assessed by C-SSRS, (m) medical history of abuse of alcohol, drugs, or medications within the last 2 years, and/or (n) clinically relevant abnormalities of routine laboratory parameters at screening.

The composition (e.g., a viral vector described herein, such as an AAV vector, e.g., an AAV vector having any one of the serotypes selected from AAV2 or AAV9) can include an inhibitory RNA sequence of the disclosure, such as a ribopolynucleotide including a nucleic acid sequence with at least 95% sequence identity to SEQ ID NO: 2 or the nucleic acid sequence of SEQ ID NO: 2, wherein the inhibitory ribopolynucleotide targets a GRIK2 mRNA. For example, the composition can include an AAV vector (e.g., AAV9 vector) with Construct B.

The subject can be administered the composition by parenteral injection, such as by administration directly to the central nervous system (e.g., stereotactic, intraparenchymal, intrathecal, or intracerebroventricular injection; in particular intraparenchymal injection). The composition can be administered to the subject in a single dose per hemisphere comprising the amount, such as by advancing a needle through the hippocampus at, for example, 5 focal sites within the hippocampus by MRI-guided convection enhanced delivery (CED) using a CLEARPOINT® Neuro System SMARTFLOW® cannula. The volume of the single dose can be divided equally by the number of focal sites (e.g., 5). The composition can be administered in a therapeutically effective amount, such as at a dose of from about 1×10¹¹ vg/mL to about 1.0×10¹³ vg/mL, in a volume of 1.8 mL or less (e.g., ˜360.0 μL per focal site). The composition can be administered to the subject in an amount of about 1×10¹¹ vg/mL, 2×10¹¹ vg/mL, 3×10¹¹ vg/mL, 4×10¹¹ vg/mL, 5×10¹¹ vg/mL, 6×10¹¹ vg/mL, 7×10¹¹ vg/mL, 8×10¹¹ vg/mL, 9×10¹¹ vg/mL, 1×10¹² vg/mL, 2×10¹² vg/mL, 3×10¹¹ vg/mL, 4×10¹² vg/mL, 5×10¹² vg/mL, 6×10¹² vg/mL, 7×10¹² vg/mL, 8×10¹² vg/mL, 9×10¹² vg/mL, or 1×10¹³ vg/mL. For example, the composition can be administered to the subject in an amount of from about 3×10⁸ vg/mm³ hippocampus to about 1.2×10⁹ vg/mm³ hippocampus. Preferably, the composition is administered to the subject in an amount of from about 9×10¹¹ total vg/hippocampus to about 3.6×10¹² total vg/hippocampus.

The subject's hippocampal volume estimate is obtained from an MRI brain volume determination, which can be utilized to calculate a precise dose of drug administered. Hippocampal volumes may also be estimated based on age range, using a published database, or by nomogram determination. The agent can be administered once and the subject can be evaluated after treatment to determine whether a subsequent dose is needed. The composition may be administered in combination with a second therapeutic modality, such as a second therapeutic agent (e.g., an anti-epileptic drug), surgical intervention (e.g., surgical resection, radiosurgery, gamma knife, or laser ablation), vagus nerve stimulation, deep brain stimulation, or transcranial magnetic stimulation.

Administration of the composition decreases one or more of (e.g., 2 or more, 3 or more, 4 or more of): (a) seizure recurrence; (b) excitotoxicity and associated neuronal cell death in the CNS; (c) one or more symptoms of a epilepsy (e.g., frequency, duration, or intensity of epileptic seizures, weakness, absence, sudden confusion, trouble understanding or producing speech, cognitive impairment, impaired mobility, dizziness, or loss of balance or coordination, paralysis, and emotional dysregulation), and (e) pathological sprouting of recurrent mossy fibers of dentate gyrus granule cells in the hippocampus by 10% or more (e.g., 10%, 20%, 30%, 40%, 50%, 60%, 70%, 80%, 90%, 95% or more). Administration of the composition also restores a physiological excitation-inhibition balance in the affected region of the CNS. In particular, administration of the composition (a) reduces the number of seizures per day and/or reduces epileptiform discharges in the subject, for example, as measured by an electroencephalogram and standardized to seizure frequency per 30 days, (b) improves the subject's measurements on routine laboratory parameters, such as hematology, biochemistry, coagulation, and urinalysis parameters, within at least 4 weeks, at least 5 weeks, at least 6 weeks, at least 7 weeks, at least 8 weeks, at least 3 months, at least 4 months, at least 5 months, at least 6 months, or at least 1 year after the composition is administered, (c) reduces frequency of interictal discharges, as measured by an electroencephalogram, (d) reduces aberrant neurological behavior by the subject, and (e) produces no adverse effects after 4 weeks.

The above-listed symptoms of epilepsy may be assessed using standard methods, such as neurological examination, electroencephalogram, magnetoencephalogram, CT scan, PET scan, fMRI scan, videography, and visual observation. Measures of epilepsy symptoms from before and after administration of the composition can be compared to evaluate the efficacy of the treatment. A finding of a reduction in the symptoms of epilepsy described above indicates that the composition has successfully treated the epilepsy in the subject.

### Example 10: Treatment of an Epilepsy in a Human Subject by Administration of a Viral Vector Encoding One or More Inhibitory Ribopolynucleotides Targeting GRIK2

A human subject experiencing seizures was intra-parenchymally administered a composition (e.g., an AAV vector encoding a ribopolynucleotide that inhibits GRIK2; e.g., a construct including the nucleic acid sequence of SEQ ID NO: 16) at a dose of 2×10¹² vg/mL in a single dose of 1.8 mL distributed over injection of 5 focal sites (e.g., 360 μl per focal site) via MRI-guided CED. The subject was monitored for 8 weeks for adverse effects and experienced no adverse effects post-administration of the composition. Additionally, the subject's symptoms (e.g., seizures, such as seizure frequency and intensity) were monitored for 4 years post-administration, and a reduction and/or amelioration in symptoms was observed beginning 4 weeks post-administration of the composition.

### Example 11: Expression of vDNA, miR38R, miR3bR and GluK2 Protein Up to Six Months In Vivo

A study was conducted to assess the stability of vDNA, miR38R, miR3bR and GluK2 protein expression levels 1 month, 3 months and 6 months after injection of Construct B at a dose of 5.0E+09 gc/hippocampus or the diluent as control.

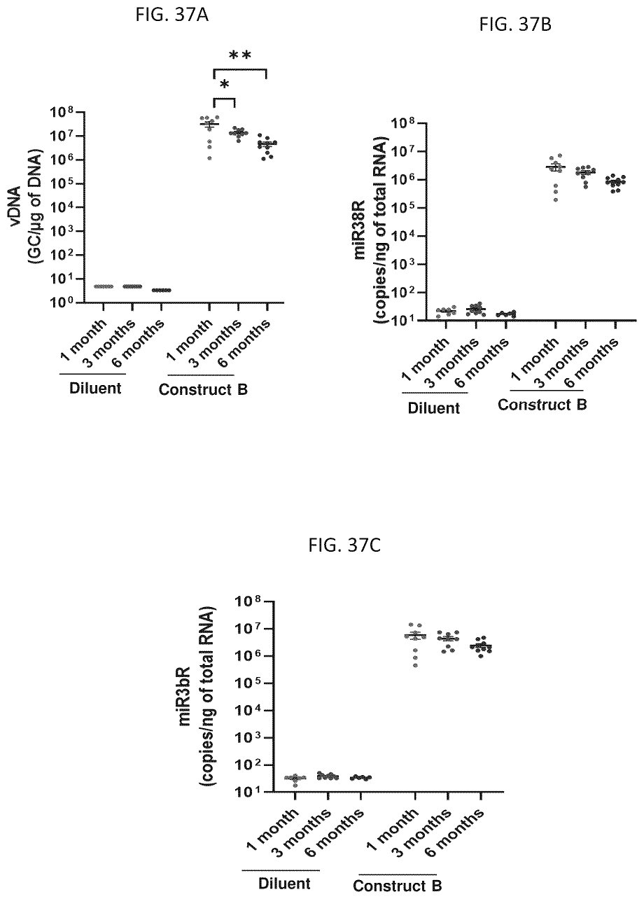

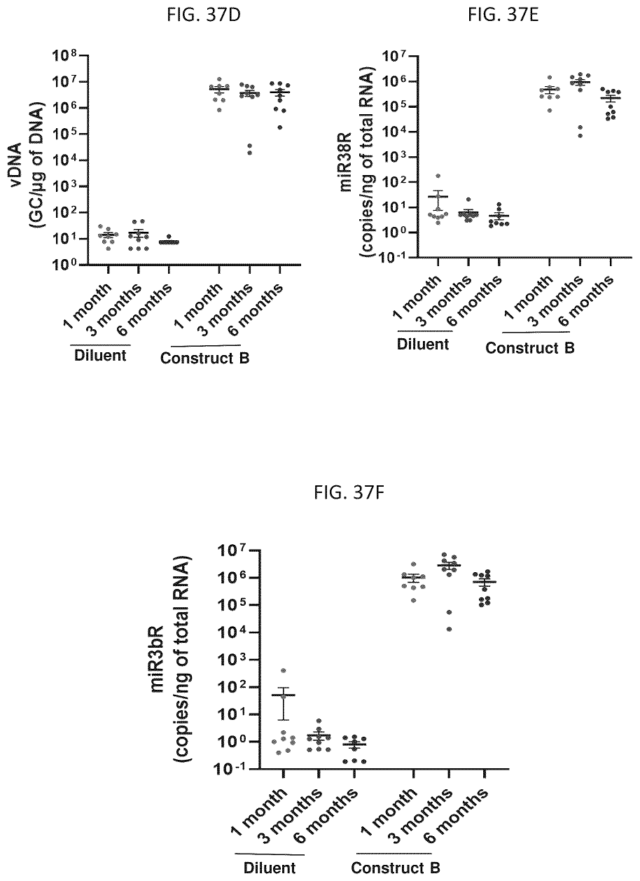

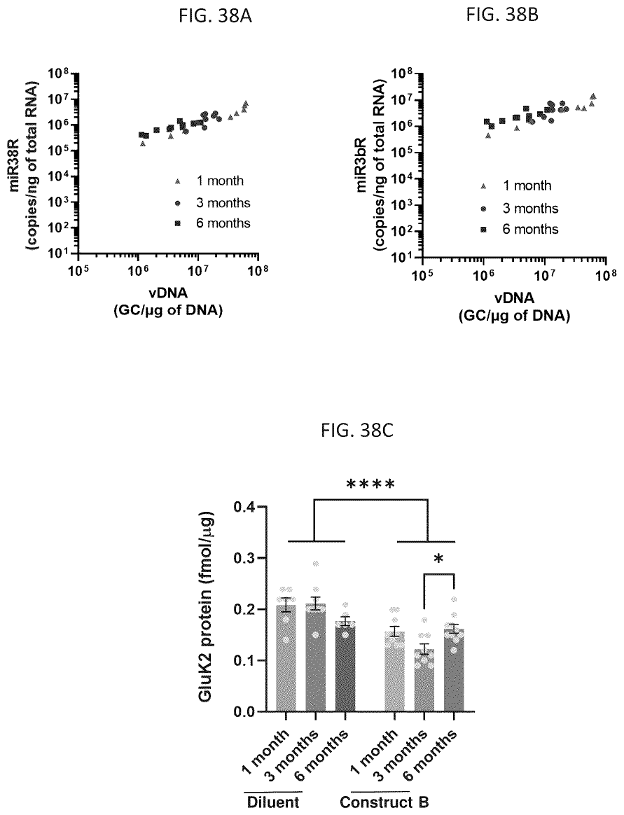

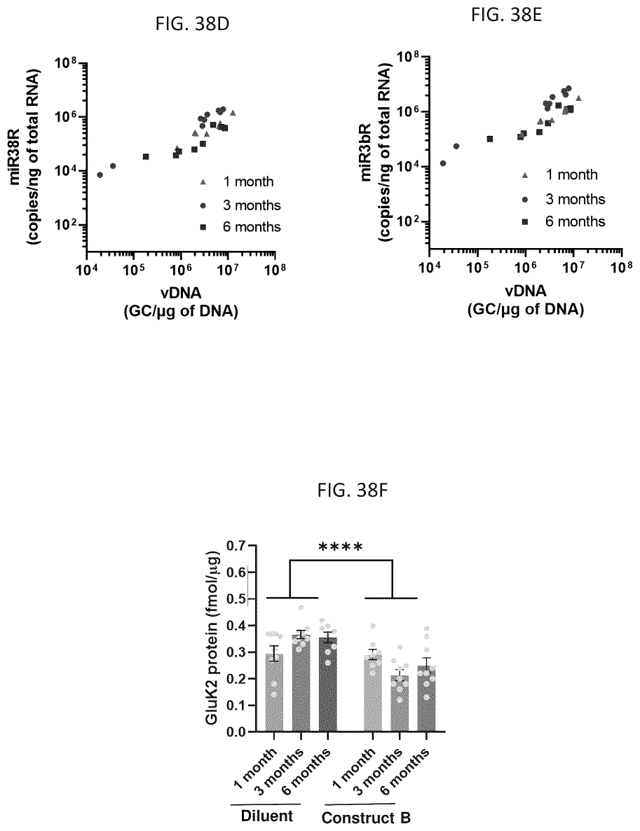

Time-course injection of Construct B in pilocarpine-treated mice showed a significant decrease of vDNA quantities between 1 month, 3 months and 6 months after injection (FIG. 37A). Expression of miR38R and miR3bR (FIGS. 37B and 37C, respectively) showed a similar pattern as the vDNA expression, such that the correlation between vDNA levels and miR38R or miR3bR remains constant over time (FIGS. 38A and 38B). The corresponding GluK2 protein expression was significantly decreased after treatment, up to 6 months (FIG. 38C).

The pilocarpine treatment has a significant effect on neuronal death in the brain, which develops over time. To control for this potential confounding factor, time-course injections of Construct B at 5.0E+09 gc/hippocampus were conducted in non-epileptic wild type mice. Amounts of vDNA and expression levels of miR38R and miR3bR were similar at 1 month, 3 months and 6 months after injection (FIGS. 37D, 37E and 37F, respectively). The correlation between vDNA levels and miR38R or miR3bR remains constant over time (FIGS. 38D and 38E). The corresponding GluK2 protein expression was significantly decreased after treatment, up to 6 months (FIG. 38F).

Altogether, these data show that the activity of the promoter remains similar from 1 month and up to 6 months after injection of construct B, resulting in a consistent knockdown of GluK2 protein expression during this timeframe.

### GLUK2 Protein Quantification by Mass Spectrometry

All solvents were HPLC-grade from Sigma-Aldrich and all chemicals where not stated otherwise were obtained from Sigma-Aldrich. Brain tissue samples were homogenized and denatured using Biognosys' Denature Buffer and a Precellys Evolution Homogenizer (Bertin Instruments). Samples were reduced and alkylated using Biognosys' Reduction and Alkylation solution. Samples were digested overnight with sequencing grade trypsin (Promega) at a protein:protease ratio of 50:1. Peptides were desalted using 96-well pHLB plate (Waters) according to the manufacturer's instructions and dried down using a SpeedVac system. Peptides were resuspended in 1% acetonitrile and 0.1% formic acid (FA) and spiked with Biognosys' iRT kit calibration peptides. Peptide concentrations in mass spectrometry ready samples were measured using the mBCA assay (THERMO SCIENTIFIC™ PIERCE™). 3 stable isotope labeled reference peptides were spiked into the final peptide samples at known concentrations (Vivitide, the quality grade of the reference peptides was ±10% quantification precision, >95% purity; purity of peptide TVTVVYDDSTGLIR (SEQ ID NO: 17) was 93.4%).

For the LC-PRM measurements, 1 μg of peptides per sample was injected to an in-house packed C18 column (PicoFrit emitter with 75 μm inner diameter, 60 cm length, and 10 μm tip from New Objective, packed with 1.7 μm Charged Surface Hybrid C18 particles from Waters) on a THERMO SCIENTIFIC™ Easy nLC 1200 nano-liquid chromatography system connected to a THERMO SCIENTIFIC™ Q EXACTIVE™ HF-X mass spectrometer equipped with a standard nano-electrospray source. LC solvents were A: 1% acetonitrile in water with 0.1% FA; B: 20% water in acetonitrile with 0.1% FA. The LC gradient was 0-59% solvent B in 54 min followed by 59-90% B in 12 sec, 90% B for 8 min (total gradient length was 67 min). A scheduled run in PRM mode was performed before data acquisition for retention time calibration using Biognosys' iRT concept (Escher, Reiter et al., Proteomics 12 (2012), 1111-1121). The data acquisition window per peptide was 6.7 minutes. Signal processing and data analysis were carried out using SpectroDive™ 11.6—Biognosys' software for multiplexed MRM/PRM data analysis based on mProphet (Reiter, Rinner et al., Nature Methods 8 (2011), 430-435). A Q-value filter of 1% was applied.

### Example 12: Proof of Mechanism and Safety in Cynomolgus Monkeys Over 6 Months

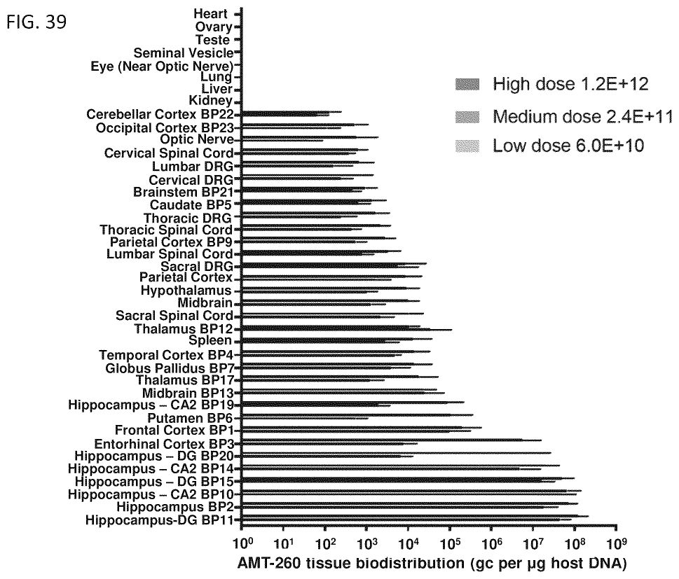

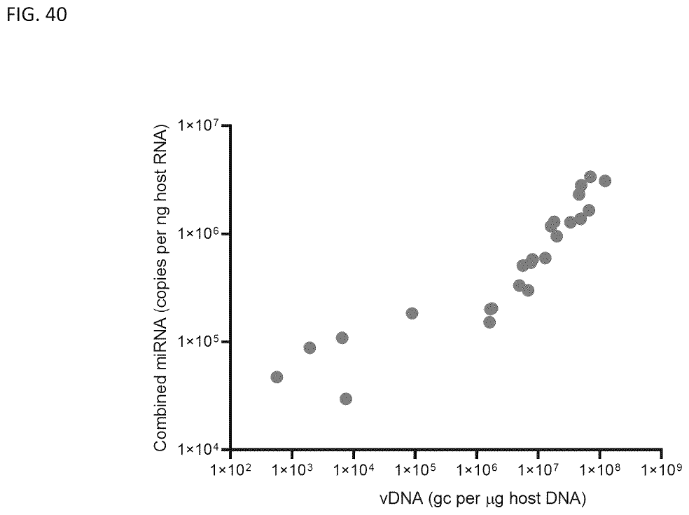

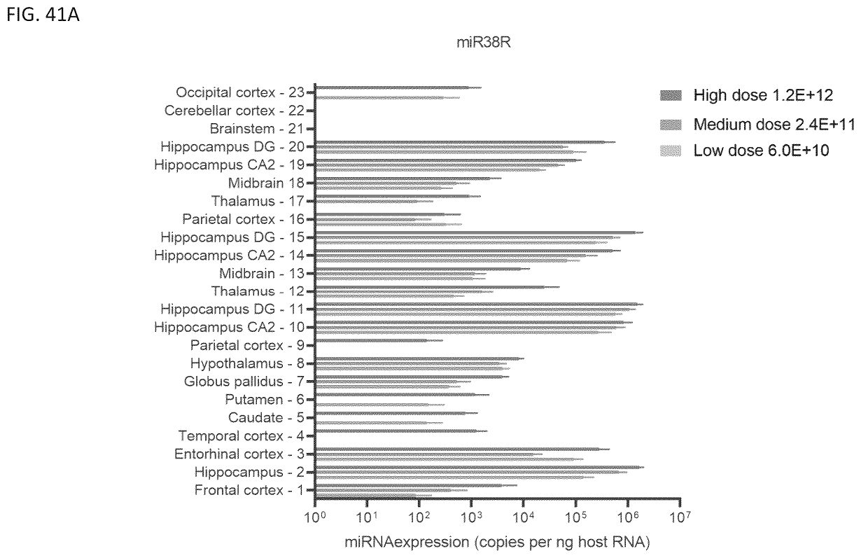

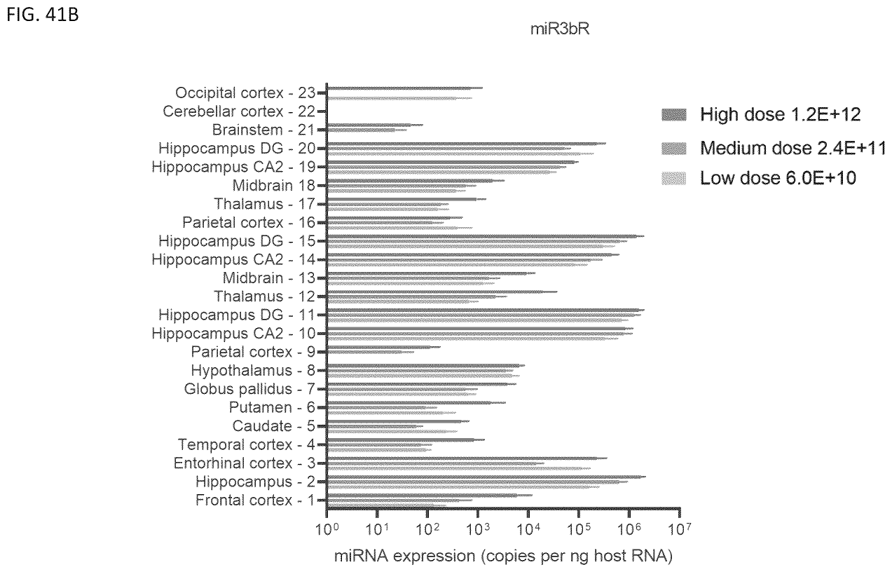

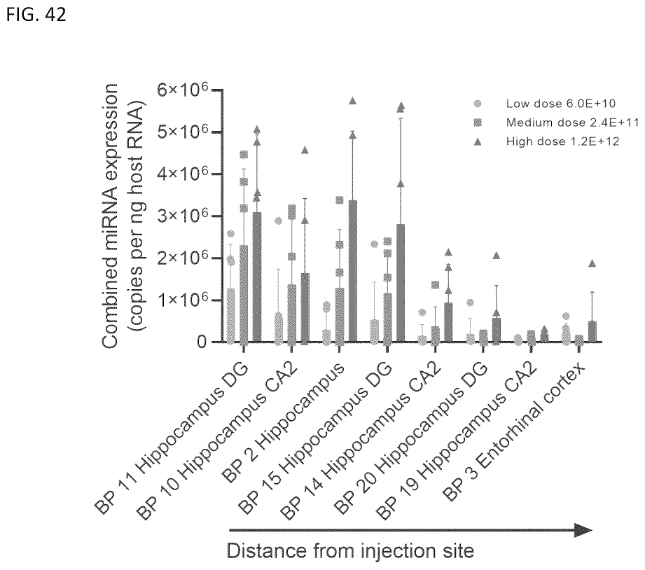

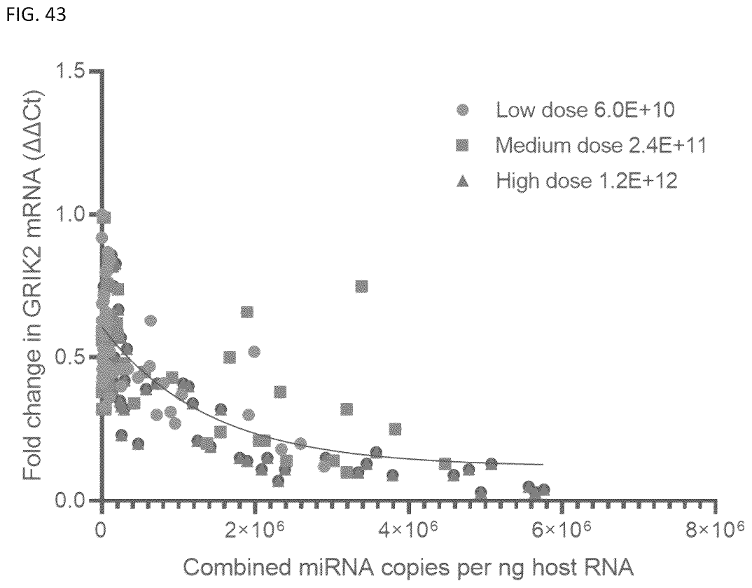

A study was conducted in Cynomolgus monkeys administered Construct B bilaterally by MRI guided convection enhanced delivery into the hippocampus at 6.0E+10, 2.4E+11 and 1.2E+12 gc/hippocampus. vDNA, miRNA and GRIK2 mRNA were assessed over 6 months. vDNA was only detected in the brain and did not transduce cells in the peripheral tissues outside the central nervous system (FIG. 39). miRNA expression of both miR38R and miR3bR was directly related to the amount of vDNA in the tissues (FIG. 40) and was highest in the target tissue, hippocampus, with lower amounts in the entorhinal cortex (a tissue adjacent to the hippocampus). All other sample punches of brain tissues had significantly lower (>100×) expression of miR38R and miR3bR (FIGS. 41A and 41B, respectively). Within the hippocampus combined miRNA expression (miR38R+miR3bR) was dose dependent and levels were highest closest to the injection site (FIG. 42). GRIK2 mRNA levels in the brain punches from the hippocampus were reduced dependent on the combined miRNA expression (FIG. 43) with as much as 99% GRIK2 knock down seen in some brain punches. Throughout the study, Construct B was well tolerated and no findings were observed at necropsy. At the clinically relevant dose of 6.0E+10 gc/hippocampus, no adverse findings were reported.

### OTHER EMBODIMENTS

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure.

Other embodiments are in the claims.

1. A composition comprising an adeno-associated virus (AAV) vector comprising a nucleic acid molecule comprising a nucleic acid sequence with at least 95% sequence identity to SEQ ID NO: 2 or 16, wherein the AAV vector is present in the composition in an amount of from about 1×10¹¹ vector genomes (vg)/mL to about 1.0×10¹³ vg/mL, the composition comprises a volume of 3.0 mL or less, and, optionally, the composition is formulated for intra-parenchymal administration.

2. The composition of claim 1, wherein the nucleic acid molecule comprises a nucleic acid sequence with at least 96% sequence identity to SEQ ID NO: 2 or 16.

3. The composition of claim 1, wherein the nucleic acid molecule comprises a nucleic acid sequence with at least 97% sequence identity to SEQ ID NO: 2 or 16.

4. The composition of claim 1, wherein the nucleic acid molecule comprises a nucleic acid sequence with at least 98% sequence identity to SEQ ID NO: 2 or 16.

5. The composition of claim 1, wherein the nucleic acid molecule comprises a nucleic acid sequence with at least 99% sequence identity to SEQ ID NO: 2 or 16.

6. The composition of claim 1, wherein the nucleic acid molecule comprises the nucleic acid sequence of SEQ ID NO: 2 or 16.

7. The composition of claim 1, wherein the nucleic acid sequence encodes an inhibitory ribopolynucleotide.

8. The composition of claim 7, wherein the inhibitory ribopolynucleotide is a dual miRNA.

9. The composition of claim 1, wherein the composition comprises a volume of 1.8 mL or less.

10. The composition of claim 1, wherein the AAV vector is an AAV9 vector.

11. A method of treating epilepsy in a subject in need thereof comprising intra-parenchymally administering the composition of claim 1 to the subject.

12. The method of claim 11, wherein administration of the composition promotes expression of a ribopolynucleotide encoded by the AAV vector in a cell of the subject transduced with the AAV vector.

13. The method of claim 12, wherein the ribopolynucleotide comprises a nucleic acid sequence of SEQ ID NO: 13.

14. The method of claim 11, wherein the epilepsy is focal epilepsy (FE) or temporal lobe epilepsy (TLE).

15. The method of claim 12, wherein administration of the composition reduces or inhibits translation of GRIK2 messenger RNA (mRNA) and/or reduces a level of Gluk2 protein in the cell of the subject, wherein the cell is present in a hippocampus of the subject.

16. The method of claim 15, wherein the translation of GRIK2 mRNA in the cell is reduced or inhibited and/or the level of Gluk2 protein is reduced for at least 28 days or at least 180 days.

17. The method of claim 15, wherein the translation of GRIK2 mRNA in the cell is reduced by at least 10% or more relative to a control composition or relative to a cell in the hippocampus of the subject that is not transduced with the AAV vector.

18. The method of claim 17, wherein the translation of GRIK2 mRNA in the cell is reduced by at least 20% or more relative to a control composition or relative to a cell in the hippocampus of the subject that is not transduced with the AAV vector.

19. The method of claim 15, wherein the level of GluK2 protein is reduced by at least 10% or more relative to a control composition or relative to a cell in the hippocampus of the subject that is not transduced with the AAV vector.

20. The method of claim 19, wherein the level of GluK2 protein is reduced by at least 20% or more relative to a control composition or relative to a cell in the hippocampus of the subject that is not transduced with the AAV vector.

21. The method of claim 11, wherein the composition is administered to the subject in an amount of about 1×10¹¹ vg/mL, 2×10¹¹ vg/mL, 3×10¹¹ vg/mL, 4×10¹¹ vg/mL, 5×10¹¹ vg/mL, 6×10¹¹ vg/mL, 7×10¹¹ vg/mL, 8×10¹¹ vg/mL, 9×10¹¹ vg/mL, 1×10¹² vg/mL, 2×10¹² vg/mL, 3×10¹¹ vg/mL, 4×10¹² vg/mL, 5×10¹² vg/mL, 6×10¹² vg/mL, 7×10¹² vg/mL, 8×10¹² vg/mL, 9×10¹² vg/mL, or 1×10¹³ vg/mL.

22. The method of claim 11, wherein the composition is administered to the subject in an amount of from about 9×10¹¹ total vg/hippocampus to about 3.6×10¹² total vg/hippocampus.

23. The method of claim 11, wherein the composition is administered to the subject in a single dose per hemisphere, wherein the composition is administered by advancing a needle through a hippocampus of the subject with injections at a number of focal sites of from one to ten within the hippocampus, and wherein a total volume of the single dose is divided by the number of the focal sites.

24. The method of claim 12, wherein the cell is a neuronal cell of a hippocampus of the subject.

25. The method of claim 24, wherein the neuronal cell of the hippocampus is a dentate granule cell (DGC) or a glutamatergic pyramidal neuron.

26. The method of claim 12, wherein expression of the ribopolynucleotide:
(a) reduces the number of seizures per day and/or reduces epileptiform discharges in the subject;
(b) reduces a frequency of interictal discharges;
(c) reduces aberrant neurological behavior by the subject; and/or
(d) produces no adverse effects 4 weeks after administration of the composition.

27. The method of claim 11, wherein the composition is administered to the subject in a volume of about 0.5 mL to 1.8 mL.

28. The method of claim 11, wherein the composition is administered once per year.

29. The method of claim 11, wherein the subject is a human subject.

30. A kit comprising a container comprising the composition of claim 1 and, optionally, a package insert comprising instructions for administering the composition to a subject.


SEQUENCE LISTING (25 sequences)

SEQ ID NO: 1 - DNA, 3103 bp, Homo sapiens
  source 1..3103: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacgacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggttgatctgaagcaattcgttgagtctgccttcgcccttgcccactctctcacctcctccccccggctctgactgactttactcccacaggtgagcggcggatgcccttctcctctggctgtaattagtcttggtttacttgtttcttttctgtggctgttgaaagccttgaggggctctggagggccctttgtgtgggggagtgcttgggggtgttgttgtgtgtgtgttggggagtctttgtgctcttctgcctgtgctgtgagtctgtggttgttgggctttgtgtctctcagtgtgttaggggagttgctggggtgtgcccttgtgtgggggggctgtaggggaacaaaggctgttgtgggtgtgtgttgggggggtgagcagggggtgtgggttttgttggctgcaaccccccctgcacccccctccctagttgctgagcatgcctgctttggtgtgggctcttatgggttggttgggcttcttgctggtgggggtggtgcaggtgggggtgctggtgggtgggctccttggctgggagggcttggggaggggttgtgcccctgagtctgtgctgttaggttgtagctcagccattgccttttttgtagagggtcagggacttcctttgtcccaaatctgtgtgagctaaatctgggaggtctctcaccccctctagtggttgggtaagtgtgtgtctgcaggaaggaaagggtgggagggccttttgtttcttctcttccccttctccctctccagccttgggctgtcttgggggatgctgcctttggggggatgggcagggtgggtttgcttctggttgtgactgtgctctagagcctctgctaacctgttctgccttcttctttttcctacagctcctgggcaattgctggttattgtgctgtctctattttggcaaagaattctctgaatttcgaccacccataatacccattaccctggtagatatagtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcagctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgaaagtagctagcgcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 2 - DNA, 3432 bp, Homo sapiens
  source 1..3432: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccaggccgtgaagccacagatgggcctgggttttatatcgagcagctgcctactgcctcggacttcaaggggctactttaggagcaattatcttgtttactaaaactgaataccttgctatctctttgatacatttttacaaagctgaattaaaatggtataaattatcacgggatccgaattcacgtttccagaacgtctgtagcttttctcctccttccctccattttcctcttggtcttacctttggcctagtggttggtgtagtgataatgtagcgagattttctgtagagcattgcagatggaccgggttgcgaggtatgagtaaacggtccatacgcaatgctccatggaacgtcacgcagctttctacagcatgacaagctgctgaggcttaaatcaggattttcctgtctctttctacaaaatcaaaatgaaaaaagagggctttttaggcatctccgagattatgtgctcgaggggatccgatctttttccctctgccaaaaattatggggacatcatgaagccccttgagcatctgacttctggctaataaaggaaatttattttcattgcaatagtgtgttggaattttttgtgtctctcactcggcggccgcatagtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcagctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgaccggccggctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 3 - DNA, 1541 bp, Homo sapiens
  source 1..1541: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgccaattgcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctcgagaaggtatattgctgttgacagtgagcgctaaaacaggcattagctatgggtagtgaagccacagatgcccatagctaatgcctgttttattgcctactgcctcggaattcaaggggctactttaggagcaattatcttgtttactaaaactgaataccttgctatctctttgatacatttttacaaagctgaattaaaatggtataaattatcacgggatccgatctttttccctctgccaaaaattatggggacatcatgaagccccttgagcatctgacttctggctaataaaggaaatttattttcattgcaatagtgtgttggaattttttgtgtctctcactcggcggccgcccgagtttaattggtttatagaactcttcaagctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 4 - skipped

SEQ ID NO: 5 - DNA, 1541 bp, Homo sapiens
  source 1..1541: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgccaattgcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctcgagaaggtatattgctgttgacagtgagcgacatgggttctccatatcgagactagtgaagccacagatggtctcgatatggagaacccatgctgcctactgcctcggaattcaaggggctactttaggagcaattatcttgtttactaaaactgaataccttgctatctctttgatacatttttacaaagctgaattaaaatggtataaattatcacgggatccgatctttttccctctgccaaaaattatggggacatcatgaagccccttgagcatctgacttctggctaataaaggaaatttattttcattgcaatagtgtgttggaattttttgtgtctctcactcggcggccgcccgagtttaattggtttatagaactcttcaagctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 6 - DNA, 3432 bp, Homo sapiens
  source 1..3432: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctaaagaaggtatattgctgttgacagtgagcgacgtctcgatatggagaacccatgctgtgaagccacagatgggcatgggttttatatcgagacgctgcctactgcctcggacttcaaggggctactttaggagcaattatcttgtttactaaaactgaataccttgctatctctttgatacatttttacaaagctgaattaaaatggtataaattatcacgggatccgaattcacgtttccagaacgtctgtagcttttctcctccttccctccattttcctcttggtcttacctttggcctagtggttggtgtagtgataatgtagcgagattttctgcagagcattgcagatggactgggttgcgaggtatgagtaaacagtccatacgcaatgctccgtggaacgtcacgcagctttctacagcatgacaagctgctgaggcttaaatcaggattttcctgtctctttctacaaaatcaaaatgaaaaaagagggctttttaggcatctccgagattatgtgctcgaggggatccgatctttttccctctgccaaaaattatggggacatcatgaagccccttgagcatctgacttctggctaataaaggaaatttattttcattgcaatagtgtgttggaattttttgtgtctctcactcggcggccgcatagtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcagctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgaccggccggctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 7 - DNA, 3121 bp, Homo sapiens
  source 1..3121: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccaggccgtgaagccacagatgggcctgggttttatatcgagcagctgcctactgcctcggacttcaaggggctactttaggagcaattatcttgtttactaaaactgaataccttgctatctctttgatacatttttacaaagctgaattaaaatggtataaattatcacgggatccgatctttttccctctgccaaaaattatggggacatcatgaagccccttgagcatctgacttctggctaataaaggaaatttattttcattgcaatagtgtgttggaattttttgtgtctctcactcggcggccgcatagtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcagctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgaccggccggctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 8 - DNA, 3066 bp, Homo sapiens
  source 1..3066: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctgaattcacgtttccagaacgtctgtagcttttctcctccttccctccattttcctcttggtcttacctttggcctagtggttggtgtagtgataatgtagcgagattttctgtagagcattgcagatggaccgggttgcgaggtatgagtaaacggtccatacgcaatgctccatggaacgtcacgcagctttctacagcatgacaagctgctgaggcttaaatcaggattttcctgtctctttctacaaaatcaaaatgaaaaaagagggctttttaggcatctccgagattatgtgctcgaggggatccgatctttttccctctgccaaaaattatggggacatcatgaagccccttgagcatctgacttctggctaataaaggaaatttattttcattgcaatagtgtgttggaattttttgtgtctctcactcggcggccgcatagtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcagctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgaccggccggctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 9 - DNA, 313 bp, Homo sapiens
  source 1..313: mol_type = other DNA
gtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccaggccgtgaagccacagatgggcctgggttttatatcgagcagctgcctactgcctcggacttcaaggggctactttaggagcaattatcttgtttactaaaactgaataccttgctatctctttgatacatttttacaaagctgaattaaaatggtataaatta

SEQ ID NO: 10 - DNA, 292 bp, Homo sapiens
  source 1..292: mol_type = other DNA
acgtttccagaacgtctgtagcttttctcctccttccctccattttcctcttggtcttacctttggcctagtggttggtgtagtgataatgtagcgagattttctgcagagcattgcagatggactgggttgcgaggtatgagtaaacagtccatacgcaatgctccgtggaacgtcacgcagctttctacagcatgacaagctgctgaggcttaaatcaggattttcctgtctctttctacaaaatcaaaatgaaaaaagagggctttttaggcatctccgagattatgtg

SEQ ID NO: 11 - DNA, 487 bp, Homo sapiens
  source 1..487: mol_type = other DNA
tctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctcgacgtttatctacaacactctgatttaatgtctatacaatcagagcattgcagatggactgcgagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgt

SEQ ID NO: 12 - DNA, 3649 bp, Homo sapiens
  source 1..3649: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctagcccgggctaggtcgactcgactagggataacagggtaattgtttgaatgaggcttcagtactttacagaatcgttgcctgcacatcttggaaacacttgctgggattacttcttcaggttaacccaacagaaggctaaagaaggtatattgctgttgacagtgagcgacgtctcgatatggagaacccatgctgtgaagccacagatgggcatgggttttatatcgagacgctgcctactgcctcggacttcaaggggctactttaggagcaattatcttgtttactaaaactgaataccttgctatctctttgatacatttttacaaagctgaattaaaatggtataaattatcacgggatccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggaggcggccgcccgagtttaattggtttatagaactcttcaagctagcactagtgaagcaattcgttgcattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagcgaattccaattggcgcgcctagcccgggctaggtcgactctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctcgacgtttatctacaacactctgatttaatgtctatacaatcagagcattgcagatggactgcgagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgtaccggtctcgagggatccgatctttttccctctgccaaaaattatggggacatcatgaagccccttgagcatctgacttctggctaataaaggaaatttattttcattgcaatagtgtgttggaattttttgtgtctctcactcggcggccgcccgagtttaattggtttatagaactcttcaagctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 13 - DNA, 622 bp, Homo sapiens
  source 1..622: mol_type = other DNA
gtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccaggccgtgaagccacagatgggcctgggttttatatcgagcagctgcctactgcctcggacttcaaggggctactttaggagcaattatcttgtttactaaaactgaataccttgctatctctttgatacatttttacaaagctgaattaaaatggtataaattatcacgggatccgaattcacgtttccagaacgtctgtagcttttctcctccttccctccattttcctcttggtcttacctttggcctagtggttggtgtagtgataatgtagcgagattttctgtagagcattgcagatggaccgggttgcgaggtatgagtaaacggtccatacgcaatgctccatggaacgtcacgcagctttctacagcatgacaagctgctgaggcttaaatcaggattttcctgtctctttctacaaaatcaaaatgaaaaaagagggctttttaggcatctccgagattatgtg

SEQ ID NO: 14 - DNA, 292 bp, Homo sapiens
  source 1..292: mol_type = other DNA
acgtttccagaacgtctgtagcttttctcctccttccctccattttcctcttggtcttacctttggcctagtggttggtgtagtgataatgtagcgagattttctgtagagcattgcagatggaccgggttgcgaggtatgagtaaacggtccatacgcaatgctccatggaacgtcacgcagctttctacagcatgacaagctgctgaggcttaaatcaggattttcctgtctctttctacaaaatcaaaatgaaaaaagagggctttttaggcatctccgagattatgtg

SEQ ID NO: 15 - DNA, 313 bp, Homo sapiens; identical to SEQ ID NO: 9
  source 1..313: mol_type = other DNA

SEQ ID NO: 16 - DNA, 3432 bp, Homo sapiens
  source 1..3432: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagatccggtcgggcccgcggtaccgtcgagaagcttgatgtgggcggagcttcgaaggggcgggcgcccgtggggcgggtcctgagtgggggcgggaccggggccggcacctgggtgaggttctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagggcgcgcctagcccgggctaggtcgactcgactagggataacagggtaattacgtttccagaacgtctgtagcttttctcctccttccctccattttcctcttggtcttacctttggcctagtggttggtgtagtgataatgtagcgagattttctgtagagcattgcagatggaccgggttgcgaggtatgagtaaacggtccatacgcaatgctccatggaacgtcacgcagctttctacagcatgacaagctgctgaggcttaaatcaggattttcctgtctctttctacaaaatcaaaatgaaaaaagagggctttttaggcatctccgagattatgtgtcacgggatccgaattcgtttgaatgaggcttcagtactttacagaatcgttgcctgcacatcttggaaacacttgctgggattacttcttcaggttaacccaacagaaggctaaagaaggtatattgctgttgacagtgagcgacttctcgatatggagaacccaggccgtgaagccacagatgggcctgggttttatatcgagcagctgcctactgcctcggacttcaaggggctactttaggagcaattatcttgtttactaaaactgaataccttgctatctctttgatacatttttacaaagctgaattaaaatggtataaattactcgaggggatccgatctttttccctctgccaaaaattatggggacatcatgaagccccttgagcatctgacttctggctaataaaggaaatttattttcattgcaatagtgtgttggaattttttgtgtctctcactcggcggccgcatagtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcagctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgaccggccggctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 17 - protein, 14 aa, Homo sapiens
TVTVVYDDSTGLIR

SEQ ID NO: 18 - RNA, 52 nt, Homo sapiens
  source 1..52: mol_type = mRNA
ggcgttggcactcccatgggttctccatatcgagacaaaattaccatagcaa

SEQ ID NO: 19 - RNA, 52 nt, Macaca fascicularis; identical to SEQ ID NO: 18
  source 1..52: mol_type = mRNA

SEQ ID NO: 20 - RNA, 52 nt, Mus musculus
  source 1..52: mol_type = mRNA
ggtgttggcactcccatgggttctccatatcgagacaaaatcaccatagcca

SEQ ID NO: 21 - RNA, 22 nt, synthetic construct
  source 1..22: mol_type = other RNA
ggacccaagaggtatagctctt

SEQ ID NO: 22 - RNA, 52 nt, Homo sapiens
  source 1..52: mol_type = mRNA
tcagcaaacgcagtgcagtccatctgcaatgctctgggagttccccacatac

SEQ ID NO: 23 - RNA, 53 nt, Macaca fascicularis
  source 1..53: mol_type = mRNA
tcagcaaatgcagtgcagtccatctgcaatgctctgggagttccccacactac

SEQ ID NO: 24 - RNA, 52 nt, Mus musculus
  source 1..52: mol_type = mRNA
tcagcaaatgctgttcagtccatctgcaatgctctgggggttcctcacatac

SEQ ID NO: 25 - RNA, 21 nt, synthetic construct
  source 1..21: mol_type = other RNA
gccaggtagacgttacgagat